(19) United States
(12) Patent Application Publication
(10) Pub. No.: US 2023/0255982 A1
(43) Pub. Date: Aug. 17, 2023

(54) METHODS OF TREATING COVID-19 USING BARDOXOLONE METHYL OR ANALOGS THEREOF

(71) Applicant: REATA PHARMACEUTICALS HOLDINGS, LLC, Plano, TX (US)

(72) Inventors: Deborah FERGUSON, Plano, TX (US); W. Christian WIGLEY, Plano, TX (US); Colin MEYER, Plano, TX (US); J. Warren HUFF, Plano, TX (US); Robert M. KRAL, Jr., Plano, TX (US)

(73) Assignee: REATA PHARMACEUTICALS HOLDINGS, LLC, Plano, TX (US)

(21) Appl. No.: 17/998,291

(22) PCT Filed: May 7, 2021
(86) PCT No.: PCT/US2021/031282

§ 371 Date: Nov. 9, 2022

Related U.S. Application Data
(60) Provisional application No. 63/022,479, filed on May 9, 2020.
(60) Provisional application No. 63/053,056, filed on Jul. 17, 2020.

(51) Int. Cl. A61K 31/56; A61K 31/675; C07K 16/28; A61P 31/14

(52) U.S. Cl. CPC A61K 31/56; A61K 31/675; C07K 16/2866; A61P 31/14; C07B 2200/13

(57) ABSTRACT

The present invention provides methods of treating patients infected with a coronavirus. In particular, provided are methods of treating or preventing COVID-19, or symptoms or complications thereof, in patients in need thereof using bardoxolone methyl or analogs thereof, and/or preventing the onset of COVID-19 in patients infected with SARS-COV-2.

### REFERENCE TO RELATED APPLICATIONS

The present application claims the priority benefit of U.S. provisional application No. 63/053,056, filed Jul. 17, 2020, and U.S. provisional application No. 63/022,479, filed May 9, 2020, the entire contents of each of which is incorporated herein by reference.

### BACKGROUND

### 1. Field

The present disclosure relates generally to the fields of medicine and biology. More particularly, it concerns, in some aspects, methods for treating or preventing COVID-19 or symptoms thereof using bardoxolone methyl and analogs thereof.

### 2. Description of Related Art

The WHO has declared the Coronavirus Disease 2019 (COVID-19) outbreak a pandemic. This virus is related to other coronaviruses that have created pandemics called the Severe Acute Respiratory Syndrome (SARS-CoV) in 2002 and the Middle East Respiratory Syndrome (MERS-CoV) in 2012. The virus that causes COVID-19 has been named SARS-CoV-2 because its share near 80% of the genome with the SARS-CoV.

Bardoxolone methyl has been shown to improve both estimated glomerular filtration rate (eGFR) and measured glomerular filtration rate (mGFR) in patients with CKD due to Type 2 diabetes. Bardoxolone methyl and several of its analogues also have been shown to inhibit pro-fibrotic signaling pathways and reduce oxidative stress and inflammation in multiple models of CKD. These compounds have also been shown to reduce proinflammatory cytokines and chemokines, prevent organ damage (lung, liver, and pancreas), and increase survival in models of systemic inflammation.

In patients with COVID-19, acute kidney injury (AKI) has been reported to occur in up to 28% of all patients and up to 72% of non-survivors. Unfortunately, there are no specific drugs or vaccines for coronaviruses. As such, new therapies to treat COVID-19 are needed.

### SUMMARY

In one aspect, the present invention provides methods of treating or preventing symptoms or complications of a coronavirus infection in a patient in need thereof. Such methods are described in the sections below, including for example the claims section, which is incorporated herein by reference.

In some embodiments, the compound is bardoxolone methyl (BARD, CDDO-Me or RTA 402). In some of these embodiments, at least a portion of the CDDO-Me is present as a polymorphic form, wherein the polymorphic form is a crystalline form having an X-ray diffraction pattern (CuKα) comprising significant diffraction peaks at about 8.8, 12.9, 13.4, 14.2 and 17.4° 20. In non-limiting examples, the X-ray diffraction pattern (CuKα) is substantially as shown in FIG. 1A or FIG. 1B.

In some of these embodiments, at least a portion of the CDDO-Me is present as a polymorphic form, wherein the polymorphic form is crystalline form having an X-ray diffraction pattern (CuKα) comprising significant diffraction peaks at about 6.2, 12.4, 15.4, 18.6 and 24.9° 20. In some aspects, the crystalline form is further characterized by one, two, three, four or five additional diffraction peaks selected from the group consisting of 8.6, 13.3, 13.7, 17.1 and 21.7° 20. In non-limited examples, the X-ray diffraction pattern (CuKα) is substantially as shown in FIG. 1 of WO 2019/014412, which is incorporated herein by reference in its entirety.

In some of these embodiments, at least a portion of the CDDO-Me is present as a polymorphic form, wherein the polymorphic form is crystalline form having an X-ray diffraction pattern (CuKα) comprising significant diffraction peaks at about 3.6, 7.1, 10.8, 12.4 and 16.5° 20. In some aspects, the crystalline form is further characterized by one, two, three, four or five additional diffraction peaks selected from the group consisting of 12.9, 13.9, 14.8, 18.6 and 20.6° 20. In non-limited examples, the X-ray diffraction pattern (CuKα) is substantially as shown in FIG. 2 of WO 2019/014412, which is incorporated herein by reference in its entirety. In some aspects, the crystalline form is further characterized by a Raman spectrum having peaks at 2949, 1671, 1618 and 1464±4 cm⁻¹. In non-limited examples, the Raman spectrum is substantially as shown in FIGS. 4 and 5 of WO 2019/014412, which is incorporated herein by reference in its entirety.

In some of these embodiments, at least a portion of the CDDO-Me is present as a polymorphic form, wherein the polymorphic form is a toluene solvate crystalline form having an X-ray diffraction pattern (CuKα) comprising diffraction peaks at about 9.65, 7.58, 7.18, 6.29, 6.06, 5.47, 5.21, 4.77 and 3.07 °2θ. In non-limited examples, the X-ray diffraction pattern (CuKα) is substantially as shown in FIG. 1 of CN102887936, which is incorporated herein by reference in its entirety.

In some of these embodiments, at least a portion of the CDDO-Me is present as a polymorphic form, wherein the polymorphic form is a semi-dioxane solvate crystalline form having an X-ray diffraction pattern (CuKα) comprising diffraction peaks at about 10.01, 7.09, 6.84, 6.23, 5.29, 5.20, 5.10, 4.84, and 4.61 °2θ. In non-limited examples, the X-ray diffraction pattern (CuKα) is substantially as shown in FIG. 4 of CN102887936, which is incorporated herein by reference in its entirety.

In some of these embodiments, at least a portion of the CDDO-Me is present as a polymorphic form, wherein the polymorphic form is a semi-tetrahydrofuran solvate crystalline form having an X-ray diffraction pattern (CuKα) comprising diffraction peaks at about 10.00, 7.14, 6.80, 6.65, 6.10, 5.62, 5.29, 4.88, and 4.50 °2θ. In non-limited examples, the X-ray diffraction pattern (CuKα) is substantially as shown in FIG. 8 of CN102887936, which is incorporated herein by reference in its entirety.

In some of these embodiments, at least a portion of the CDDO-Me is present as a polymorphic form, wherein the polymorphic form is a methanol solvate crystalline form having an X-ray diffraction pattern (CuKα) comprising diffraction peaks at about 8.86, 8.45, 8.17, 7.90, 7.26, 4.67, 6.63, 6.46, and 3.64 °2θ. In non-limited examples, the X-ray diffraction pattern (CuKα) is substantially as shown in FIG. 1 of CN102875634, which is incorporated herein by reference in its entirety.

In some of these embodiments, at least a portion of the CDDO-Me is present as a polymorphic form, wherein the polymorphic form is an anhydrous crystalline form having an X-ray diffraction pattern (CuKα) comprising diffraction peaks at about 12.05, 8.90, 8.49, 8.13, 7.92, 7.29, 6.64, 4.67 and 3.65 °2θ. In non-limited examples, the X-ray diffraction pattern (CuKα) is substantially as shown in FIG. 2 of CN102875634, which is incorporated herein by reference in its entirety.

In some of these embodiments, at least a portion of the CDDO-Me is present as a polymorphic form, wherein the polymorphic form is a dihydrate crystalline form having an X-ray diffraction pattern (CuKα) comprising diffraction peaks at about 8.81, 8.48, 7.91, 7.32, 5.09, 4.24, 3.58, 3.36 and 3.17 °2θ. In non-limited examples, the X-ray diffraction pattern (CuKα) is substantially as shown in FIG. 3 of CN102875634, which is incorporated herein by reference in its entirety.

In some of these embodiments, at least a portion of the CDDO-Me is present as a polymorphic form, wherein the polymorphic form is an amorphous form having an X-ray diffraction pattern (CuKα) with a halo peak at approximately 13.5 °2θ, substantially as shown in FIG. 1C, and a T_{g}. In some variations, the compound is an amorphous form. In some variations, the compound is a glassy solid form of CDDO-Me, having an X-ray powder diffraction pattern with a halo peak at about 13.5 °2θ, as shown in FIG. 1C, and a T_{g}. In some variations, the T_{g} value falls within a range of about 120° C. to about 135° C. In some variations, the T_{g} value is from about 125° C. to about 130° C.

In some embodiments, the compound is administered locally. In some embodiments, the compound is administered systemically. In some embodiments, the compound is administered orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, orally, parenterally, rectally, subconjunctivally, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof. For example, in some variations, the compound is administered intravenously, intra-arterially or orally. For example, in some variations, the compound is administered orally.

In some embodiments, the compound is formulated as a hard or soft capsule, a tablet, a syrup, a suspension, a solid dispersion, a wafer, or an elixir. In some variations, the soft capsule is a gelatin capsule. In variations, the compound is formulated as a solid dispersion. In some variations the hard capsule, soft capsule, tablet or wafer further comprises a protective coating. In some variations, the formulated compound comprises an agent that delays absorption. In some variations, the formulated compound further comprises an agent that enhances solubility or dispersibility. In some variations, the compound is dispersed in a liposome, an oil-in-water emulsion or a water-in-oil emulsion.

In some embodiments, the pharmaceutically effective amount is a daily dose from about 0.1 mg to about 500 mg of the compound. In some variations, the daily dose is from about 1 mg to about 300 mg of the compound. In some variations, the daily dose is from about 10 mg to about 200 mg of the compound. In some variations, the daily dose is about 25 mg of the compound. In other variations, the daily dose is about 75 mg of the compound. In still other variations, the daily dose is about 150 mg of the compound. In further variations, the daily dose is from about 0.1 mg to about 30 mg of the compound. In some variations, the daily dose is from about 0.5 mg to about 20 mg of the compound. In some variations, the daily dose is from about 1 mg to about 15 mg of the compound. In some variations, the daily dose is from about 1 mg to about 10 mg of the compound. In some variations, the daily dose is from about 1 mg to about 5 mg of the compound. In some variations, the daily dose is from about 2.5 mg to about 30 mg of the compound. In some variations, the daily dose is about 2.5 mg of the compound. In other variations, the daily dose is about 5 mg of the compound. In other variations, the daily dose is about 10 mg of the compound. In other variations, the daily dose is about 15 mg of the compound. In other variations, the daily dose is about 20 mg of the compound. In still other variations, the daily dose is about 30 mg of the compound.

In some embodiments, the pharmaceutically effective amount is a daily dose of 0.01-25 mg of compound per kg of body weight. In some variations, the daily dose is 0.05-20 mg of compound per kg of body weight. In some variations, the daily dose is 0.1-10 mg of compound per kg of body weight. In some variations, the daily dose is 0.1-5 mg of compound per kg of body weight. In some variations, the daily dose is 0.1-2.5 mg of compound per kg of body weight.

In some embodiments, the pharmaceutically effective amount is administered in a single dose per day. In some embodiments, the pharmaceutically effective amount is administered in two or more doses per day.

In some embodiments, the patient is a mammal such as primate. In some variations, the primate is a human. In other variations, the patient is a cow, horse, dog, cat, pig, mouse, rat or guinea pig.

In some variations of the above methods, the compound is substantially free from optical isomers thereof. In some variations of the above methods, the compound is in the form of a pharmaceutically acceptable salt. In other variations of the above methods, the compound is not a salt.

In some embodiments, the compound is formulated as a pharmaceutical composition comprising (i) a therapeutically effective amount of the compound and (ii) an excipient selected from the group consisting of (A) a carbohydrate, carbohydrate derivative, or carbohydrate polymer, (B) a synthetic organic polymer, (C) an organic acid salt, (D) a protein, polypeptide, or peptide, and (E) a high molecular weight polysaccharide. In some variations, the excipient is a synthetic organic polymer. In some variations, the excipient is selected from the group consisting of a hydroxypropyl methyl cellulose, a poly[1-(2-oxo-1-pyrrolidinyl)ethylene] or copolymer thereof, and a methacrylic acid-methylmethacrylate copolymer. In some variations, the excipient is hydroxypropyl methyl cellulose phthalate ester. In some variations, the excipient is PVP/VA. In some variations, the excipient is a methacrylic acid-ethyl acrylate copolymer. In some variations, the methacrylic acid and ethyl acrylate may be present at a ratio of about 1:1. In some variations, the excipient is copovidone.

Any embodiment discussed herein with respect to one aspect of the invention applies to other aspects of the invention as well, unless specifically noted.

Further aspects and embodiments of this invention are elaborated in greater detail, for example, in the claims section and in the examples section, which are incorporated herein by reference.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula does not mean that it cannot also belong to another generic formula.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C—X-ray Powder Diffraction (XRPD) Spectra of Forms A and B of RTA 402. FIG. 1A shows unmicronized Form A; FIG. 1B shows micronized Form A; FIG. 1C shows Form B.

FIG. 2—Schematic of Study Design for Phase II and Phase III.

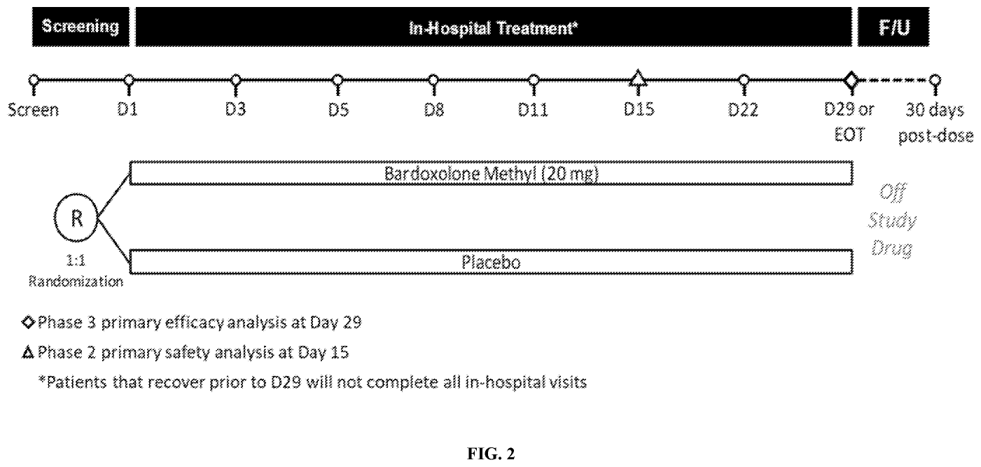

### DETAILED DESCRIPTION

In one aspect, the present invention provides new methods for treating or preventing COVID-19, or a symptom or complication thereof, or preventing the onset of symptoms resulting from a SAR-CoV-2 infection in patients, using bardoxolone methyl and analogs thereof.

Bardoxolone methyl and analogues exhibit potent anti-inflammatory activity in vitro. Moreover, bardoxolone and analogues suppress inflammation and tissue damage in animal models of acute lung injury and reduce mortality in models of systemic inflammation. In addition to the anti-inflammatory and tissue protective effects, bardoxolone methyl and analogues have been shown to possess potent antiviral activity. AKI is a serious complication of COVID-19 and frequently occurs in patients with severe symptoms. Bardoxolone methyl protects the kidney in several animal models of CKD and AKI and improves kidney function in patients with diabetes, Alport syndrome, ADPKD, IgAN, and FSGS. Thus, the collective data suggest bardoxolone methyl may reduce the excessive production of cytokines and chemokines and prevent ARDS and AKI in patients with COVID-19.

Some patients with moderate-to-severe COVID-19 rapidly develop symptoms that lead to serious complications such as ARDS, AKI, and multiple organ failure. The severity of COVID-19, as well as the development of serious complications, is associated with an imbalance in the response of the immune system to the infection with excessive production of pro-inflammatory cytokines and chemokines. Provided herein are compounds that may be used to reduce pro-inflammatory cytokines and/or chemokines in a patient infected with a coronavirus (e.g., a beta-coronavirus; e.g., SARS-CoV-2). Provided herein are compounds that may be used to treat or prevent acute respiratory distress syndrome in a patient infected with a coronavirus (e.g., a beta-coronavirus; e.g., SARS-CoV-2). Provided herein are compounds that may be used to treat or prevent acute kidney injury in a patient infected with a coronavirus (e.g., a beta-coronavirus; e.g., SARS-CoV-2). Provided herein are compounds that may be used to treat or prevent multiple organ failure in a patient infected with a coronavirus (e.g., a beta-coronavirus; e.g., SARS-CoV-2).

These and other aspects of the invention are described in greater detail below.

### I. SARS-CoV-2 and COVID-19

Coronavirus infections include infections with virus of the genera Alphacoronavirus, Betacoronavirus (which includes Severe acute respiratory syndrome-related coronavirus (SARS-CoV), SARS-CoV-2, and Middle East respiratory syndrome-related coronavirus (MERS-CoV), Gammacorouavirus, and Deltacoronavirus. The disclosure provides compositions comprising a compound of the present disclosure useful for treating coronavirus infections and methods of treating these infections by administering the compound to a patient infected with the virus.

The WHO has declared the Coronavirus Disease 2019 (COVID-19) outbreak a pandemic. This virus is related to other coronaviruses that have created pandemics called the Severe Acute Respiratory Syndrome (SARS-CoV) in 2002 and the Middle East Respiratory Syndrome (MERS-CoV) in 2012. The virus that causes COVID-19 has been named SARS-CoV-2 because its share near 80% of the genome with the SARS-CoV.

Following viral infection of a cell, viral RNA is detected by pattern recognition receptors (PRRs). TLR 3, TLR7, TLR8, and TLR9 sense viral RNA (and DNA) in endosomes. RIG-I, MDAS, and cGAS sense viral RNA (and DNA) in the cytoplasm. As part of the innate immune response, PRRs recruit adaptors, including TRIF, MAVS, and STING, and activate NF-κB and IRF3, leading to the production of type I interferons (IFNα/β) and a series of pro-inflammatory cytokines and chemokines. Innate and adaptive immune cells are recruited and activated including CD8⁺-specific cytotoxic T cells, CD4⁺ helper T cells, and antigen-specific B-cells. This adaptive immune response controls the viral infection and determines clinical recovery.

Like SARS-CoV-1, the SARS-CoV-2 (COVID-19) virus enters alveolar epithelial cells by binding the angiotensin-converting enzyme-2 (ACE-2), causing the formation of endosomes and the release of viral RNA (Ahmadppor & Rostaing, 2020). Type I interferon inhibits viral replication and promotes T cell stimulation, differentiation, expansion, which leads to killing of virus-infected cells. Highly pathogenic human coronaviruses often encode viral proteins with the capability of antagonizing type I interferon production (Fung et al., 2020; Sun et al., 2012; Chen et al., 2014). The exact mechanisms by which SARS-CoV-2 (COVID-19) might counteract host antiviral defense remain to be elucidated (Fung et al., 2020). When the body is unable to produce an adequate adaptive response against the virus, the persistent innate-induced inflammation can then lead to a cytokine storm, acute respiratory distress syndrome (ARDS) (Potey et al., 2019; Kellner et al., 2017), acute kidney injury (AKI), and multiple organ failure (Sarzi-Puttini, 2020; Huang, 2020; Chen, 2020; Guan, 2020). Provided herein are compounds that may be used to treat or prevent a cytokine storm in a patient infected with a coronavirus (e.g., a beta-coronavirus; e.g., SARS-CoV-2). Provided herein are compounds that may be used to treat or prevent acute respiratory distress syndrome in a patient infected with a coronavirus (e.g., a beta-coronavirus; e.g., SARS-CoV-2). Provided herein are compounds that may be used to treat or prevent acute kidney injury in a patient infected with a coronavirus (e.g., a beta-coronavirus; e.g., SARS-CoV-2). Provided herein are compounds that may be used to treat or prevent multiple organ failure in a patient infected with a coronavirus (e.g., a beta-coronavirus; e.g., SARS-CoV-2).

Mechanical lung ventilation is a standard therapeutic method for ARDS patients. However, mechanical ventilation itself can increase lung inflammation and worsen clinical condition due to ventilation-induced lung injury (VILI) (Kellner et al., 2017). Cyclic stretch, which occurs as a result of mechanical ventilation, in the bronchial epithelium increases the production of reactive oxygen species (Chapman et al., 2005). Loss of Nrf2, a key regulator of inflammation and oxidative stress, increased susceptibility to ventilation-induced lung injury, and activation of Nrf2 is protective in many models of lung injury (Papaiahgari et al., 2017; Zhao et al., 2017). Therefore, mechanical lung ventilation, although medically necessary, may exacerbate the production of cytokines and reactive oxygen species that are a key feature of ARDS. Provided herein are compounds that may be used to reduce lung inflammation in a patient infected with a coronavirus (e.g., a beta-coronavirus; e.g., SARS-CoV-2). Provided herein are compounds that may be used to reduce the production of reaction oxygen species in a patient infected with a coronavirus (e.g., a beta-coronavirus; e.g., SARS-CoV-2). Provided herein are compounds that may be used to induce activation of Nrf2 in a patient infected with a coronavirus (e.g., a beta-coronavirus; e.g., SARS-CoV-2).

With regard to other organ involvement, the SARS-CoV-2 cellular receptor, angiotensin-converting enzyme 2 (ACE2), is also present on cells in the heart, blood vessels, kidney, neural cortex, and brain stem. Patients infected with SARS-CoV-2 have increased incidents of blood clots, heart attacks, cardiac inflammation, strokes, seizures, brain inflammation, and kidney damage, including acute kidney injury. The effects seen on various organs may result from direct infection by SARS-CoV-2 or from systemic complications of SARS-CoV-2 infection, such as inflammation. Provided herein are compounds that may be used to treat or prevent blood clots in a patient infected with a coronavirus (e.g., a beta-coronavirus; e.g., SARS-CoV-2). Provided herein are compounds that may be used to treat or prevent heart attacks in a patient infected with a coronavirus (e.g., a beta-coronavirus; e.g., SARS-CoV-2). Provided herein are compounds that may be used to treat or prevent cardiac inflammation in a patient infected with a coronavirus (e.g., a beta-coronavirus; e.g., SARS-CoV-2). Provided herein are compounds that may be used to treat or prevent strokes in a patient infected with a coronavirus (e.g., a beta-coronavirus; e.g., SARS-CoV-2). Provided herein are compounds that may be used to treat or prevent seizures in a patient infected with a coronavirus (e.g., a beta-coronavirus; e.g., SARS-CoV-2). Provided herein are compounds that may be used to treat or prevent brain inflammation in a patient infected with a coronavirus (e.g., a beta-coronavirus; e.g., SARS-CoV-2). Provided herein are compounds that may be used to treat or prevent kidney damage (e.g., acute kidney injury) in a patient infected with a coronavirus (e.g., a beta-coronavirus; e.g., SARS-CoV-2).

Many complications of infectious diseases also involve dysregulation of inflammatory responses (e.g., cytokine storm). Although an inflammatory response can kill invading pathogens, an excessive inflammatory response can also be quite destructive and in some cases can be a primary source of damage in infected tissues. Furthermore, an excessive inflammatory response can also lead to systemic complications due to overproduction of inflammatory cytokines, such as TNF-α and IL-1. Cytokine storm involves excessive production of pro-inflammatory cytokines and chemokines, is associated with lung injury, and predicts disease severity (Yang et al., 2020; Liu et al., 2020). Table 1 provides a summary of cytokines found to be elevated in COVID-19. Provided herein are compounds that may be used to reduce or suppress the production of inflammatory cytokines and/or chemokines in a patient infected with a coronavirus (e.g., a beta-coronavirus; e.g., SARS-CoV-2).

TABLE 1
Cytokines elevated in COVID-19

| | Infected vs | ICU/Severe vs | |
|---|---|---|---|
| Patients | Healthy | Non-ICU/Severe | Reference |
| Wuhan, China | IL-6, IL-10, and | IL-6, IL-10, and | Diao et al., medRxiv, |
| Total N = 522 | TNFα | TNFα | 2020.2002.2018.20024364 |
| ICU (N = 43), Non- | | | |
| ICU (N = 479) | | | |
| Healthy controls | | | |
| (N = 40) | | | |
| Wuhan, China | IL-2R, IL-6, and | IL-2R, IL-6, IL-8, | Qin et al., Clin Infect Dis, |
| Total N = 452 | TNFα | IL-10, and TNFα | 2020 |
| Severe (N = 286), | | | doi: 10.1093/cid/ciaa248 |
| Non-Severe | | | |
| (N = 166) | | | |
| Wuhan, China | CTACK, G-CSF, | IL-1RA, IP-10, | Yang et al., medRxiv, |
| Total N = 53 | IFN-γ, IL-1RA, IL- | MCP-3 | 2020.2003.2002.20029975 |
| Severe (N = 34), | 2RA, IL-6, IL-10, | | |
| Moderate (N = 19) | IL-18, IP-10, HGF, | | |
| Healthy controls | MCP-3, M-CSF, | | |
| (N = 8) | MIG, and MIG-1a | | |
| Wuhan, China | Basic FGF, G-CSF, | G-CSF, IL-2, IL-7, | Huang et al., Lancet |
| Total N = 41 | GM-CSF, IL-1B, | IL-10, IP-10, MCP- | 2020; 395: 497-506 |
| ICU (N = 13), Non- | IL-1RA, IL-7, IL- | 1, MIP-1A, and | |
| ICU (N = 28) | 8, IL-9, IL-10, | TNFα | |
| Healthy controls | IFNγ, IP-10, MCP- | | |
| (N = 4) | 1, MIP-1A, MIP- | | |
| | 1B, PDGF, TNFα, | | |
| | and VEGF | | |
| Wuhan, China | N/A | IL-2R, IL-6, IL-10, | Chen et al., J Clin Invest |
| Total N = 21 | | and TNF-α | 2020; |
| Severe (N = 11), | | | doi: 10.1172/JCI137244. |
| Moderate (N = 10) | | | |
| Shenzhen, China | 38 of 48 cytokines | G-CSF, IFN-α2 and | Liu et al., |
| Total N = 12 | elevated | IFN-γ, IL-1RA, IL- | chinaXiv: 202002.00018v1 |
| Healthy controls | | 2, IL-4, IL-7, IL-10, | |
| (N = 8) | | IL-12 and | |
| | | IL-17, IP-10, and | |
| | | M-CSF were | |
| | | associated with | |
| | | disease severity | |

The Keap1-Nrf2 system rapidly responds to cellular stress by orchestrating an elaborate genetic program that enhances cellular cytoprotective functions, including detoxification, antioxidant, and anti-inflammatory networks (Dinkova-Kostova, 2015). Bardoxolone methyl and related analogues activate the Keap1-Nrf2 system, which allows Nrf2 to increase expression of antioxidant and cytoprotective genes and decrease expression of pro-inflammatory NF-κB target genes (Lee, 2009; Dinkova-Kostova, 2005; Rojas-Rivera, 2012; Osburn & Kensler, 2008). Consistent with this activity, bardoxolone methyl and analogues suppress proinflammatory cytokines and chemokines and reduce oxidative stress in many cell types in response to a variety of inflammatory triggers (Chen 2015; Thimmulappa, 2007; Pei, 2019, Nichols, 2009). Approximately 3200 individuals have been exposed to bardoxolone methyl in clinical trials, including studies in patients with cancer, chronic kidney disease (CKD), and pulmonary hypertension (PH).

The potent anti-inflammatory effects of bardoxolone methyl in cultured cells has translated into broad protective activity in animal models of acute lung injury and inflammation (Nichols, 2009, Chen 2015, Pei, 2019; Reddy, 2009; Zhang, 2019; Nagashima, 2019; Kulkarni, 2013). Bardoxolone methyl and analogs significantly reduce neutrophil and macrophage infiltration and suppress proinflammatory cytokine and chemokine levels in the lungs of mice treated with inflammatory stimuli (Nichols, 2009; Chen 2015, Reddy, 2009). In these models, bardoxolone methyl and analogs also reduced pulmonary edema, decreased lung injury scores, prevented fibrosis, and improved lung function (Chen 2015; Pei, 2019; Kulkarni, 2013). Bardoxolone methyl and analogues also reduce proinflammatory cytokines and chemokines, prevent organ damage (lung, liver, and pancreas), and increase survival in models of systemic inflammation (Thimmulappa, 2006; Auletta, 2010, Osburn, 2008; Keleku-Lukwete, 2015; Robles, 2016). Provided herein are compounds that may be used to reduce neutrophil and/or macrophage infiltration in a patient infected with a coronavirus (e.g., a beta-coronavirus; e.g., SARS-CoV-2).

In addition to their anti-inflammatory and tissue protective activity, bardoxolone methyl and analogs have been shown to inhibit viral replication, suppress viral infection, inhibit transcription of viral genes, and prevent latent viral reactivation in vitro (Vázquez, 2005; Shao, 2016; Chandra, 2018; Patra, 2019; Nio, 2019; Wyler, 2019; Rothan, 2019). Consistent with the mechanism of action of these compounds, the Nrf2 target heme oxygenase-1 (H0-1) has been shown to exhibit significant antiviral activity (Espinoza, 2017). Provided herein are compounds that may be used to inhibit viral replication in a patient infected with a coronavirus (e.g., a beta-coronavirus; e.g., SARS-CoV-2). Provided herein are compounds that may be used to inhibit transcription of viral genes in a patient infected with a coronavirus (e.g., a beta-coronavirus; e.g., SARS-CoV-2).

Lastly, preclinical studies have demonstrated that bardoxolone methyl and analogues protect kidney tissue, reduce inflammation, prevent fibrosis, and increase kidney function in many different animal models of kidney disease, including: ischemia-reperfusion-induced acute kidney injury (AKI) (Liu, 2014), chemically induced acute kidney injury (AKI) (Tanaka, 2008; Aleksunes, 2010; Wu, 2014), CKD associated with diabetes and/or obesity (Chin, 2013; Tan, 2014; Camer, 2016), CKD caused by nephron loss (Aminzadeh, 2013; Aminzadeh, 2014; Son, 2015), CKD caused by glomerulonephritis (Nagasu, 2019), autoimmune-associated kidney disease (Wu, 2014), and hypertension-associated kidney disease (Hisamichi, 2018). Provided herein are compounds that may be used to protect kidney tissue in a patient infected with a coronavirus (e.g., a beta-coronavirus; e.g., SARS-CoV-2). Provided herein are compounds that may be used to increase kidney function in a patient infected with a coronavirus (e.g., a beta-coronavirus; e.g., SARS-CoV-2). Provided herein are compounds that may be used to treat or prevent fibrosis in a patient infected with a coronavirus (e.g., a beta-coronavirus; e.g., SARS-CoV-2). Provided herein are compounds that may be used to treat or prevent chronic kidney disease in a patient infected with a coronavirus (e.g., a beta-coronavirus; e.g., SARS-CoV-2).

Approximately 3200 individuals have been exposed to bardoxolone methyl in clinical trials, including studies in healthy subjects, patients with cancer, pulmonary hypertension (PH), and various forms of chronic kidney disease (CKD). Bardoxolone methyl has been shown to improve kidney function—assessed using a variety of measures including measured inulin clearance, creatinine clearance, and estimated glomerular filtration rate—in patients with CKD due to diabetes, Alport syndrome, autosomal dominant polycystic kidney disease (ADPKD), IgA nephropathy (IgAN), focal segmental glomerular sclerosis (FSGS), cancer, and pulmonary hypertension (PH) (Pergola, 2011; Pergola, 2019; de Zeeuw, 2013) (Table 2). The clinical activity of bardoxolone methyl in various forms of CKD with distinct etiologies suggests that the anti-inflammatory and anti-fibrotic effects of bardoxolone methyl target the common final pathway contributing to GFR loss in multiple forms of CKDs. Provided herein are compounds that may be used to increase measured inulin clearance in a patient infected with a coronavirus (e.g., a beta-coronavirus; e.g., SARS-CoV-2). Provided herein are compounds that may be used to reduce serum creatinine levels in a patient infected with a coronavirus (e.g., a beta-coronavirus; e.g., SARS-CoV-2). Provided herein are compounds that may be used to increase the estimated glomerular filtration rate in a patient infected with a coronavirus (e.g., a beta-coronavirus; e.g., SARS-CoV-2).

Bardoxolone methyl was originally considered for development in cancer patients, and in two Phase 1 studies, bardoxolone methyl was observed to reduce serum creatinine levels, corresponding to an increase in eGFR (Hong, 2012). The reductions of serum creatinine concentrations and resultant increases in eGFR were time-dependent and manifested in a majority (82%) of the patients studied. In subsequent studies that enrolled over 2600 patients with type 2 diabetes and CKD, bardoxolone methyl has been shown to consistently produce clinically and statistically significant improvements in eGFR that are durable for at least one year in treated patients (Chin, 2018; Pergola, 2011). The change in serum creatinine was not related to a reduction in creatinine generation (Chertow, 2018); improved kidney function was confirmed in a study of Japanese patients with type 2 diabetes wherein GFR was measured using inulin clearance, as well as estimated, using conventional GFR estimating equations (Nangaku, 2020).

TABLE 2
Cross-Study Comparison of Increases in eGFR, Inulin Clearance,
and Creatinine Clearance with Bardoxolone Methyl Treatment

| | | | | | |
|---|---|---|---|---|---|
| | Phase/ | Study | # of | Treatment | ΔeGFR |
| Study | Country | Population | Patients | Duration | (mL/min/1.73 m²)^{1.} |

CKD Studies

| 402-C-0801 | 2a/ | Age ≥ 18, | 60 | 28 | days | 6.7 (p < |
|---|---|---|---|---|---|---|
| (Stratum 1) | US | Diabetic | | | | 0.001) |
| (open label) | | nephropathy | | | | |
| 402-C-0801 | 2b/ | Age ≥ 18, | 20 | 56 | days | 7.2 (p < |
| (Stratum 2) | US | Diabetic | | | | 0.001) |
| (open label) | | nephropathy | | | | CrCl also sig. |
| | | | | | | increased |
| 402-C-0804 | 2/ | Age ≥ 18, | 227 | 52 | weeks | 8.6 at WK 52 |
| (BEAM) | US | T2D and CKD | | | | (p < 0.001 vs |
| | | | | | | PBO) |
| 402-C-0902 | 2/ | Age ≥ 18, T2D and | 131 | 85 | days | 6.5 (p < |
| | US | CKD | | | | 0.001) |
| 402-C-0903 | 3/ | Age ≥ 18, | 2185 | Median: | | 6.4 (p < |
| (BEACON) | Global | T2D and Stage 4 | | 7 months with | | 0.001 vs PBO) |
| | | CKD | | 522 patients | | CrCl also sig |
| | | | | through Week | | increased |
| | | | | 48 | | |
| 402-C-1102 | 1/ | Age ≥ 18, | 24 | 56 | days | 9.0 (p < |
| | US | T2D and Stage 3b | | | | 0.05) |
| | | and Stage 4 CKD | | | | |
| RTA402- | 2/ | Age ≥ 20, | 108 | 16 | weeks | 6.6 (inulin GFR) |
| 005 | Japan | T2D and Stage 3 | | | | (p = 0.008 vs |
| (TSUBAKI) | | and 4 CKD | | | | PBO) |
| 402-C-1603 | 2/ | Age 12 to 65, | 30 | 48 | weeks | 10.4 (p < |
| | US | Alport Syndrome | | | | 0.001) |
| 402-C-1603 | 3/ | Age 12 to 70, | 157 | 48 | weeks | 9.5 (p < |
| | Global | Alport Syndrome | | | | 0.001 vs PBO) |
| 402-C-1702 | 2/ | Age 18 to 70, | 31 | 12 | weeks | 9.3 (p < |
| | US | ADPKD | | | | 0.001) |
| 402-C-1702 | 2/ | Age 18 to 70, | 26 | 12 | weeks | 8.0 (p < |
| | US | IgA Nephropathy | | | | 0.001) |
| 402-C-1702 | 2/ | Age 18 to 70, | 28 | 12 | weeks | 5.5 (p = |
| | US | TID CKD | | | | 0.02) |

Non-CKD Studies

| 402-C-0501 | 1/ | Age ≥ 18, | 47 | Median: 56 | | 18.2 (p < |
|---|---|---|---|---|---|---|
| | US | Advanced Solid | | days | | 0.0001) |
| | | Tumors or | | | | |
| | | Lymphoid | | | | |
| | | Malignancies | | | | |
| 402-C-0702 | 1/2/ | Pancreatic Cancer | 34 | Median: 56 | | 32.2 (p = |
| | US | | | days | | 0.001) |
| 402-C-1302 | 2/ | Age 18 to 75 | 542 | 16 | weeks | 14.7 (p < |
| (LARIAT) | US | PH (Baseline eGFR | | | | 0.001 vs PBO) |
| | | 82 mL/min/1.73 m²) | | | | |

^{a} Unless noted, data are mean eGFR changes from baseline for bardoxolone methyl patients and p-values are calculated from two-sided paired t-tests comparing eGFR change to 0.
^{b} Number of patients enrolled in Cohorts 1 and 2.

In patients with COVID-19, AKI has been reported to occur in up to 28% of all patients and up to 72% of non-survivors (Fanelli, 2020; Yang, 2020; Zhou, 2020; Naicker, 2020). Bardoxolone methyl and analogues protect kidney tissue, reduce inflammation, prevent fibrosis, and increase kidney function in animal models of chronic kidney disease and AKI (Chin, 2013; Tanaka, 2008; Wu, 2011; Aminzadeh, 2013).

The profile of eGFR increases with bardoxolone methyl reflects its multiple protective and anti-inflammatory effects. Early improvements in eGFR evident within the first 4 weeks of bardoxolone methyl treatment are likely attributed to the reversal of acute, dynamic inflammation-mediated processes such as endothelial dysfunction and mesangial cell contraction resulting in glomerular filtration surface area increases (Aminzadeh, 2013; Chin, 2018; Ding, 2013; Ferguson, 2010). Provided herein are compounds that may be used to treat or prevent endothelial dysfunction and/or conditions associated with endothelial dysfunction in a patient infected with a coronavirus (e.g., a beta-coronavirus; e.g., SARS-CoV-2).

### II. Compounds for the Treatment of COVID-19, or Symptoms or Complications Thereof, or the Prevention of Symptoms or Complications of COVID-19

In one aspect of the present disclosure, there are provided methods of treating or reducing the symptoms of COVID-19 in a patient comprising administering to the patient a therapeutically effective amount of bardoxolone methyl, an analog thereof, or a composition comprising either bardoxolone methyl or an analog thereof. Analogs of bardoxolone methyl include compounds of the formula:

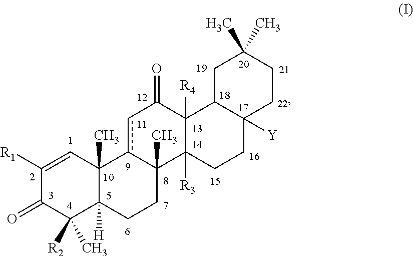

wherein:
R₁ is —CN, halo, —CF₃, or —C(O)Rₐ, wherein Rₐ is —OH, alkoxy_{(C1-4)}, —NH₂, alkylamino_{(C1-4)}, or —NH—S(O)₂-alkyl_{(C1-4)};
R₂ is hydrogen or methyl;
R₃ and R₄ are each independently hydrogen, hydroxy, methyl or as defined below when either of these groups is taken together with group R_{c}; and
Y is:
   —H, —OH, —SH, —CN, —F, —CF₃, —NH₂ or —NCO;
   alkyl_{(C≤8)}, cycloalkyl_{(C≤8)}, alkenyl_{(C≤8)}, alkynyl_{(C≤8)}, aryl_{(C≤12)}, aralkyl_{(C≤12)}, heteroaryl_{(C≤8)}, heterocycloalkyl_{(C≤12)}, alkoxy_{(C≤8)}, cycloalkoxy_{(C≤8)}, aryloxy_{(C≤12)}, acyloxy_{(C≤8)}, alkylamino_{(C≤8)}, cycloalkylamino_{(C≤8)}, dialkylamino_{(C≤8)}, arylamino_{(C≤8)}, aralkylamino_{(C≤8)}, alkylthio_{(C≤8)}, acylthio_{(C≤8)}, alkylsulfonylamino_{(C≤8)}, cycloalkylsulfonylamino_{(C≤8)}, or substituted versions of any of these groups;
   -alkanediyl_{(C≤8)}-R_{b}, -alkenediyl_{(C≤8)}-R_{b}, or a substituted version of any of these groups, wherein R_{b} is:
      hydrogen, hydroxy, halo, amino or mercapto; or
      heteroaryl_{(C≤8)}, alkoxy_{(C≤8)}, cycloalkoxy_{(C≤8)}, alkenyloxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, aralkylamino_{(C≤8)}, heteroarylamino_{(C≤8)}, alkyl sulfonylamino_{(C≤8)}, cycloalkylsulfonylamino_{(C≤8)}, amido_{(C≤8)}, —OC(O)NH-alkyl_{(C≤8)}, or a substituted version of any of these groups;
   —(CH₂)ₘC(O)R_{c}, wherein m is 0-6 and R_{c} is:
      hydrogen, hydroxy, halo, amino, —NHOH, or mercapto; or
      alkyl_{(C≤8)}, cycloalkyl_{(C≤8)}, alkenyl_{(C≤8)}, alkynyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, cycloalkoxy_{(C≤8)}, alkenyloxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, alkyl sulfonylamino_{(C≤8)}, cycloalkylsulfonylamino_{(C≤8)}, amido_{(C≤8)}, —NH-alkoxy_{(C≤8)}, —NH-heterocycloalkyl_{(C≤8)}, —NH-amido_{(C≤8)}, or a substituted version of any of these groups;
      R_{c} and R₃, taken together, are —O— or —NR_{d}—, wherein R_{d} is hydrogen or alkyl_{(C≤4)}; or
      R_{c} and R₄, taken together, are —O— or —NR_{d}—, wherein R_{d} is hydrogen or alkyl_{(C≤4)}; or
   —NHC(O)Re, wherein Re is:
      hydrogen, hydroxy, amino; or
      alkyl_{(C≤8)}, cycloalkyl_{(C≤8)}, alkenyl_{(C≤8)}, alkynyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, cycloalkoxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, or a substituted version of any of these groups;
         or a pharmaceutically acceptable salt thereof.

These compounds are known as antioxidant inflammation modulators. These compounds have shown the ability to activate Nrf2, as measured by elevated expression of one or more Nrf2 target genes (e.g., NQO1 or HO-1; Dinkova-Kostova et al., 2005). Further, these compounds are capable of indirect and direct inhibition of pro-inflammatory transcription factors including NF-κB and STAT3 (Ahmad et al., 2006; Ahmad et al., 2008). In some aspects, there are provided methods of preventing progression of COVID-19 or a symptom or complication thereof in a subject or patient in need thereof comprising administering to the subject or patient bardoxolone methyl or an analog thereof in an amount sufficient to prevent progression of COVID-19 or a symptom or complication thereof in the subject or patient. Additionally, one or more of the compounds described herein may be used in methods to prevent the onset of one or more symptoms of COVID-19 or prevent the progression of COVID-19.

Triterpenoids, biosynthesized in plants by the cyclization of squalene, are used for medicinal purposes in many Asian countries; and some, such as ursolic and oleanolic acid, are known to be anti-inflammatory and anti-carcinogenic (Huang et al., 1994; Nishino et al., 1988). However, the biological activity of these naturally occurring molecules is relatively weak, and therefore the synthesis of new analogs to enhance their potency was undertaken (Honda et al., 1997; Honda et al., 1998). An ongoing effort for the improvement of anti-inflammatory and antiproliferative activity of oleanolic and ursolic acid analogs led to the discovery of 2-cyano-3,12-dioxooleane-1,9(11)-dien-28-oic acid (CDDO) and related compounds (Honda et al., 1997, 1998, 1999, 2000a, 2000b, 2002; Suh et al., 1998; 1999; 2003; Place et al., 2003; Liby et al., 2005). Several potent derivatives of oleanolic acid were identified, including methyl-2-cyano-3,12-dioxooleana-1,9-dien-28-oic acid (CDDO-Me; RTA 402; bardoxolone methyl). RTA 402, an antioxidant inflammation modulator (AIM), suppresses the induction of several important inflammatory mediators, such as iNOS, COX-2, TNFα, and IFNγ, in activated macrophages, thereby restoring redox homeostasis in inflamed tissues. RTA 402 has also been reported to activate the Keap1/Nrf2/ARE signaling pathway resulting in the production of several anti-inflammatory and antioxidant proteins, such as heme oxygenase-1 (HO-1). It induces the cytoprotective transcription factor Nrf2 and suppresses the activities of the pro-oxidant and pro-inflammatory transcription factors NF-κB and STAT3. In vivo, RTA 402 has demonstrated significant single agent anti-inflammatory activity in several animal models of inflammation such as renal damage in the cisplatin model and acute renal injury in the ischemia-reperfusion model. In addition, significant reductions in serum creatinine have been observed in patients treated with RTA 402.

Accordingly, in pathologies involving oxidative stress alone or oxidative stress exacerbated by inflammation, treatment may comprise administering to a subject or patient a therapeutically effective amount of a compound of this invention, such as those described above or throughout this specification. Treatment may be administered preventively in advance of a predictable state of oxidative stress (e.g., organ transplantation or the administration of therapy to a cancer patient), or it may be administered therapeutically in settings involving established oxidative stress and inflammation.

Non-limiting examples of triterpenoids that may be used in accordance with the methods of this invention are shown here.

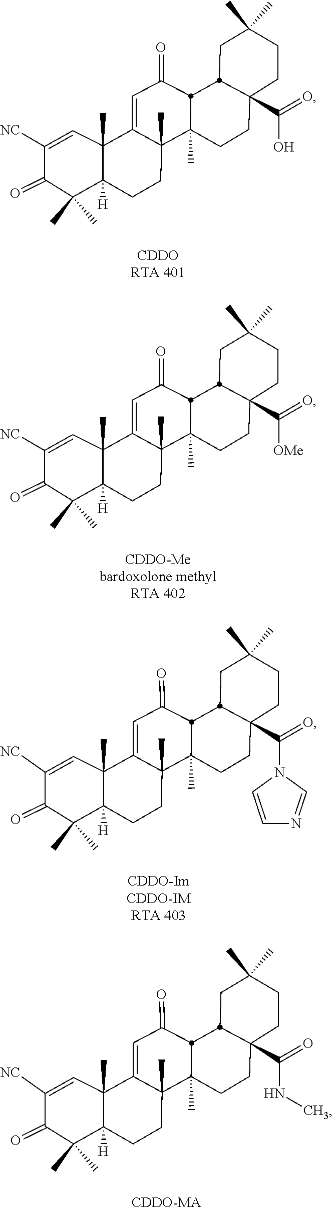

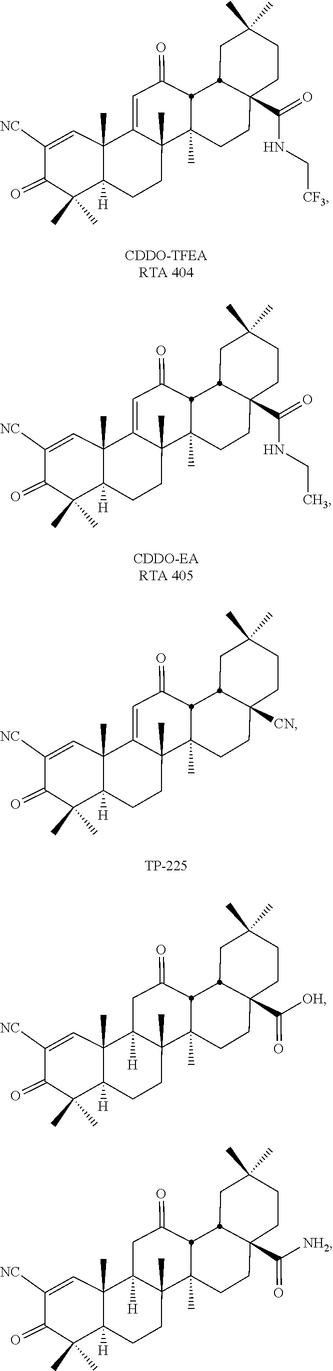

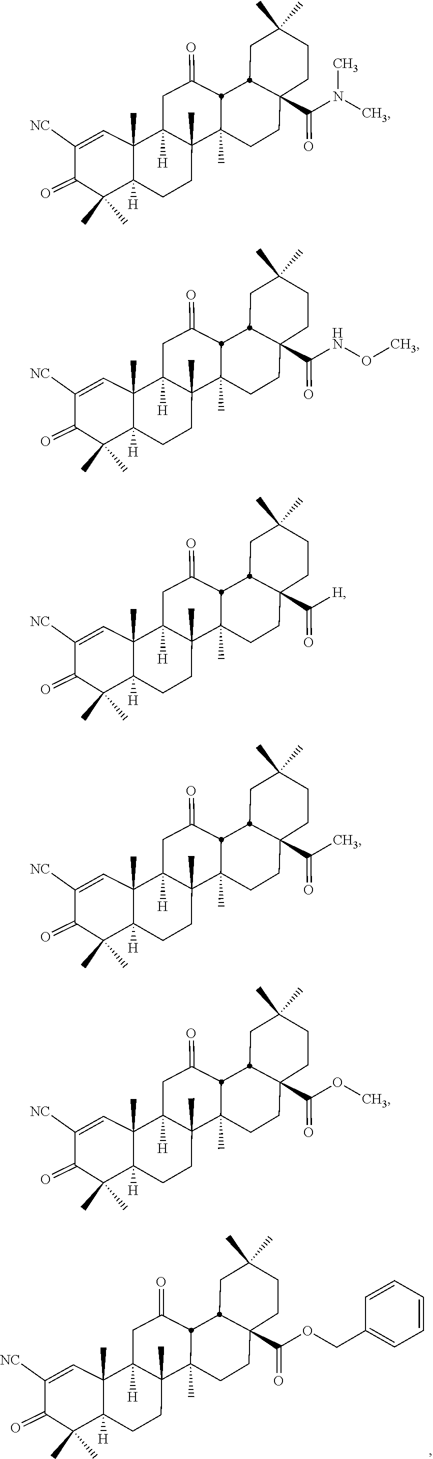

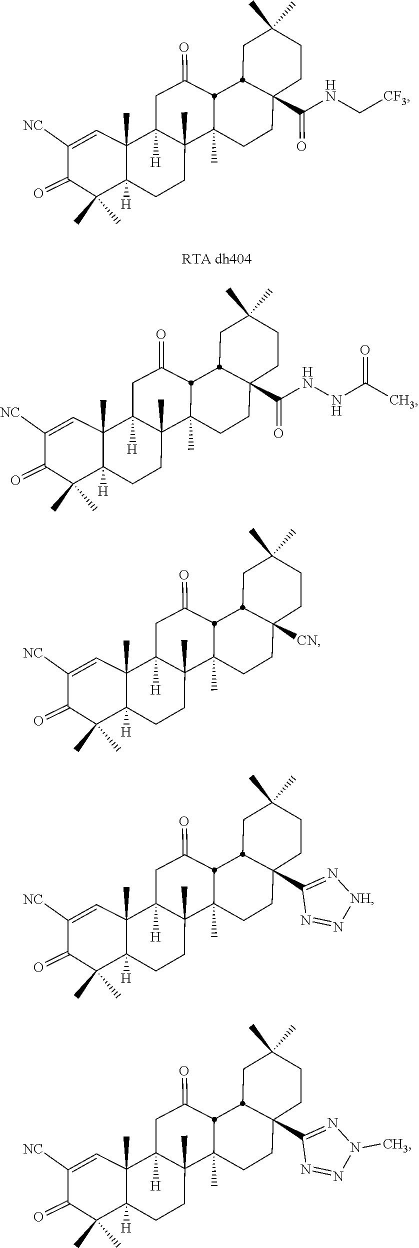

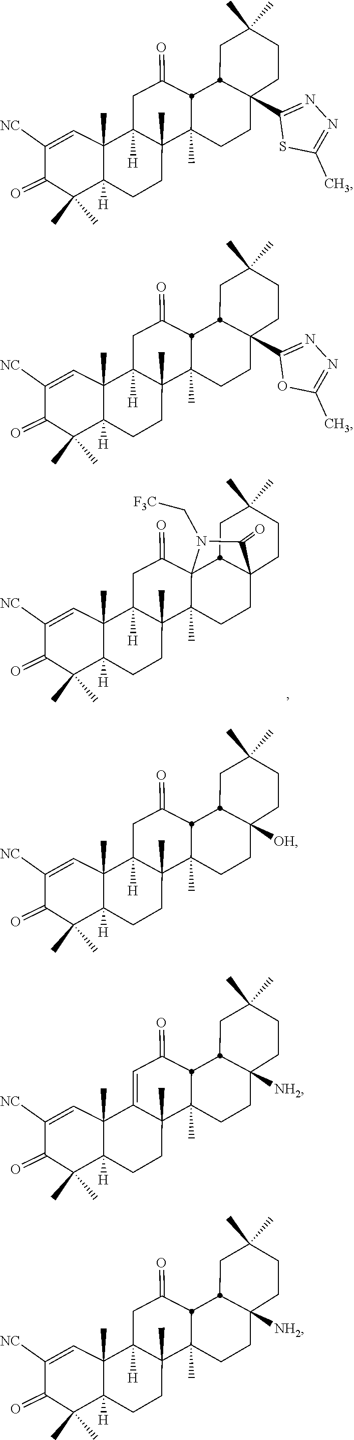

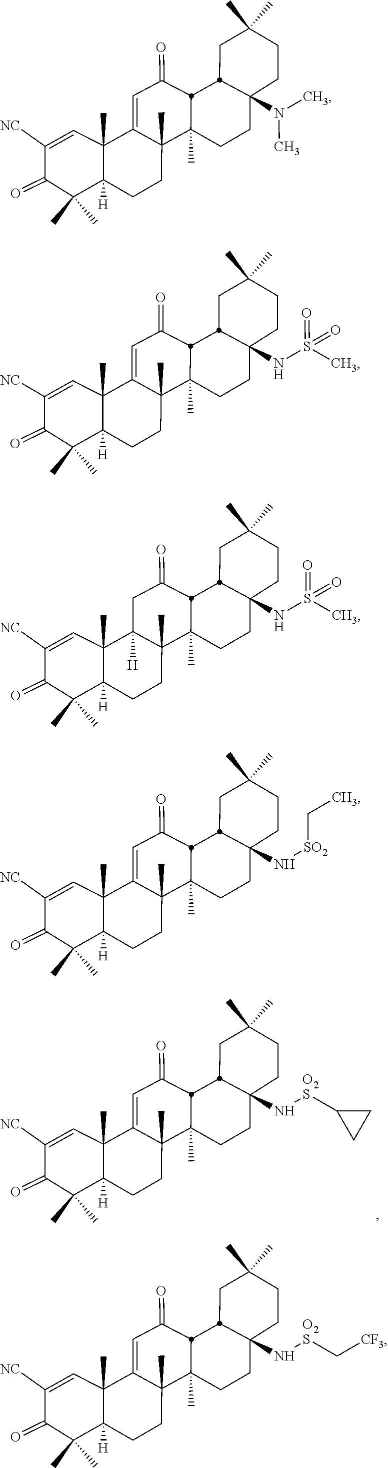

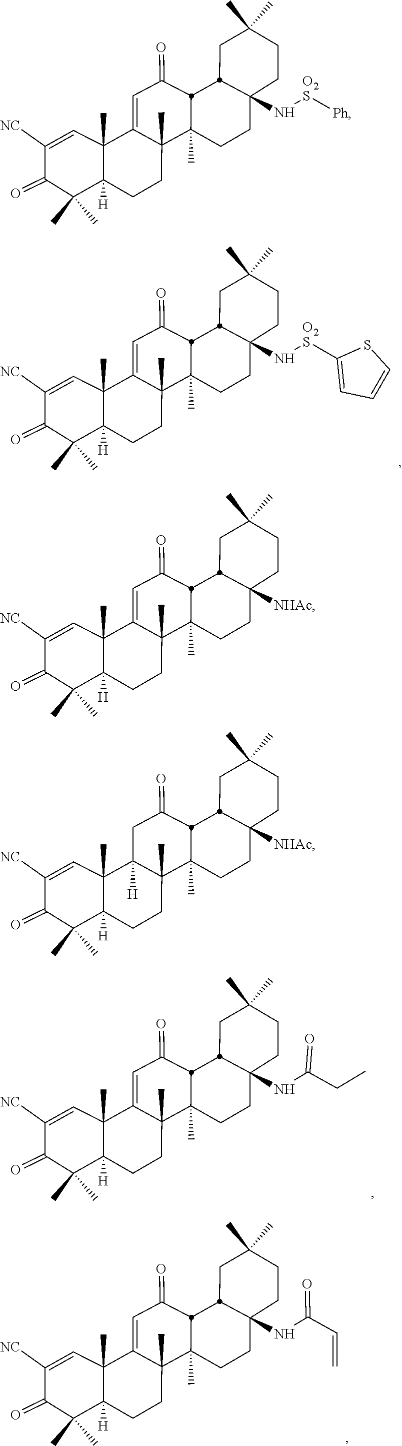

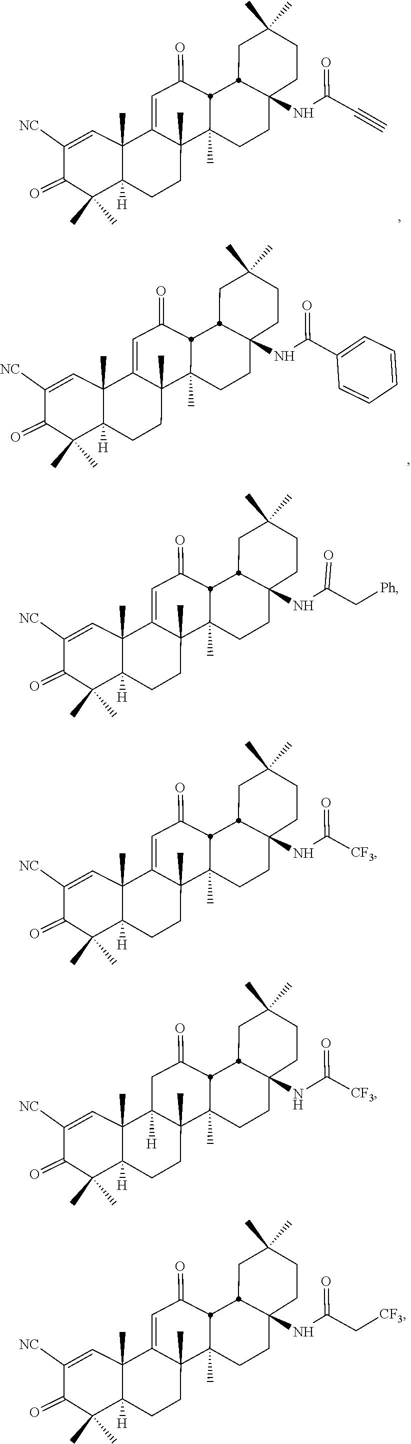

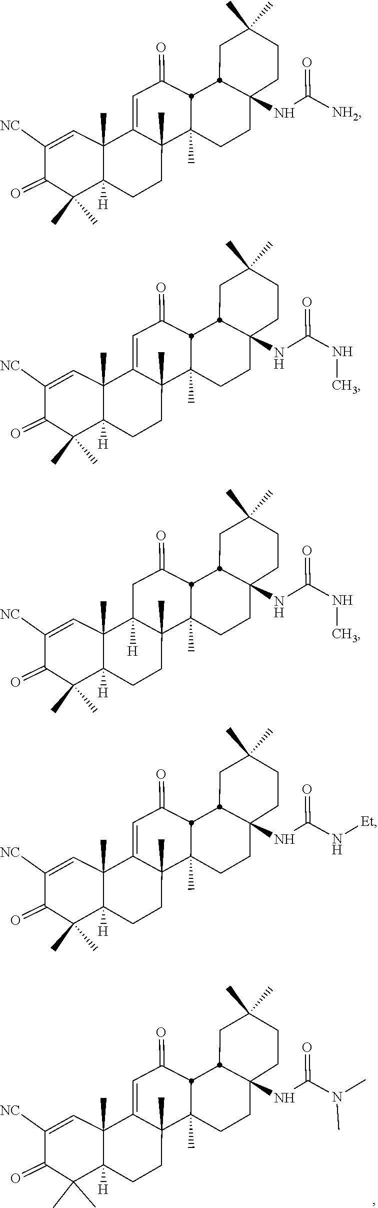

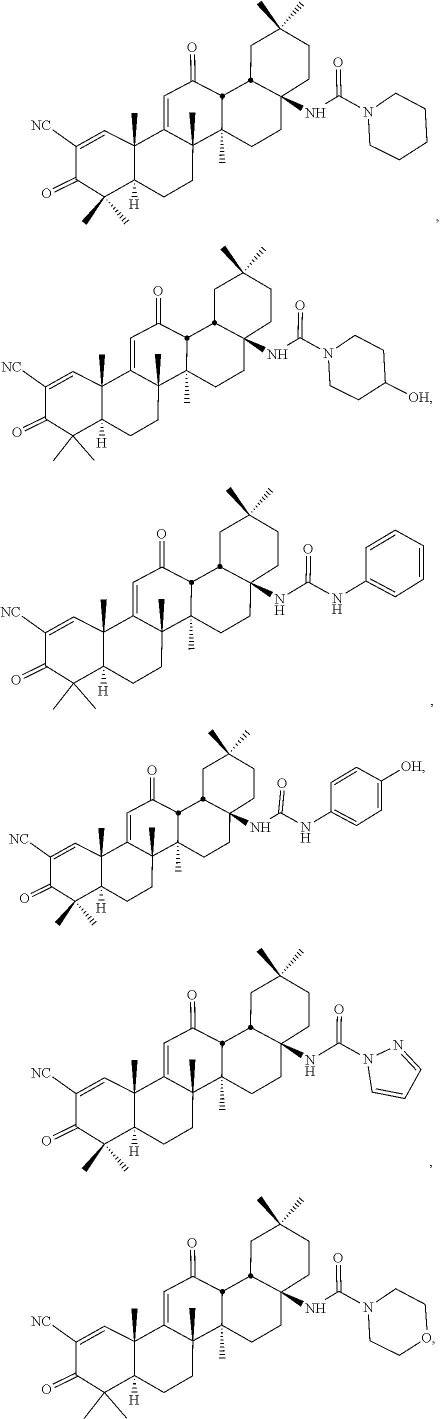

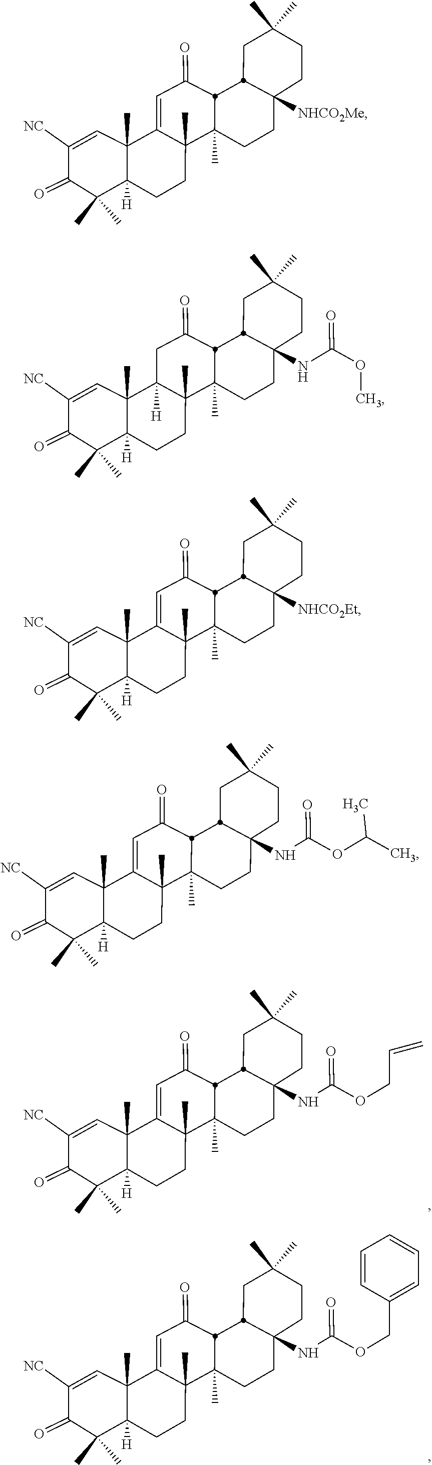

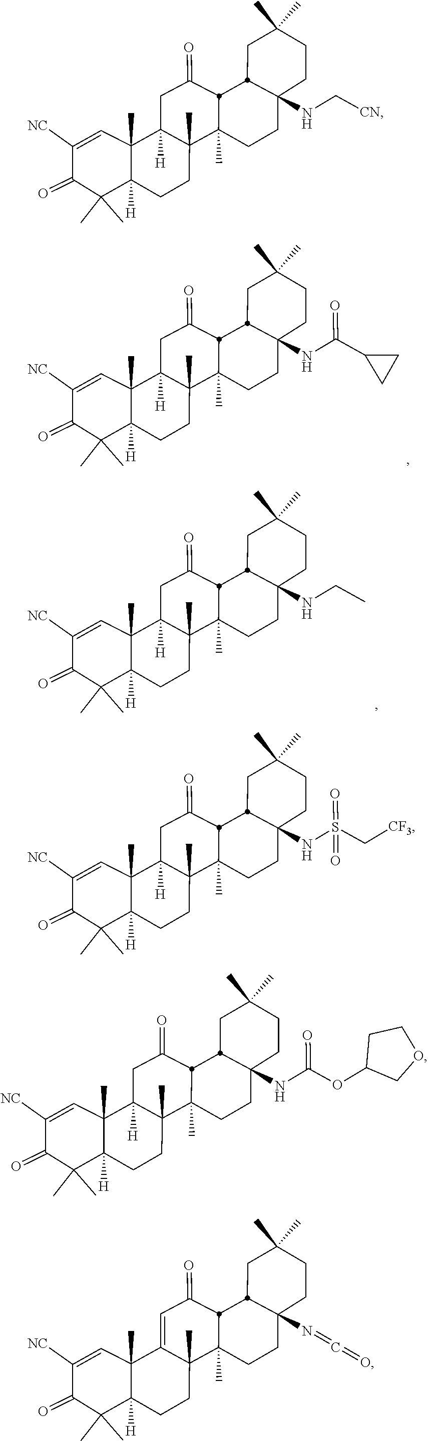

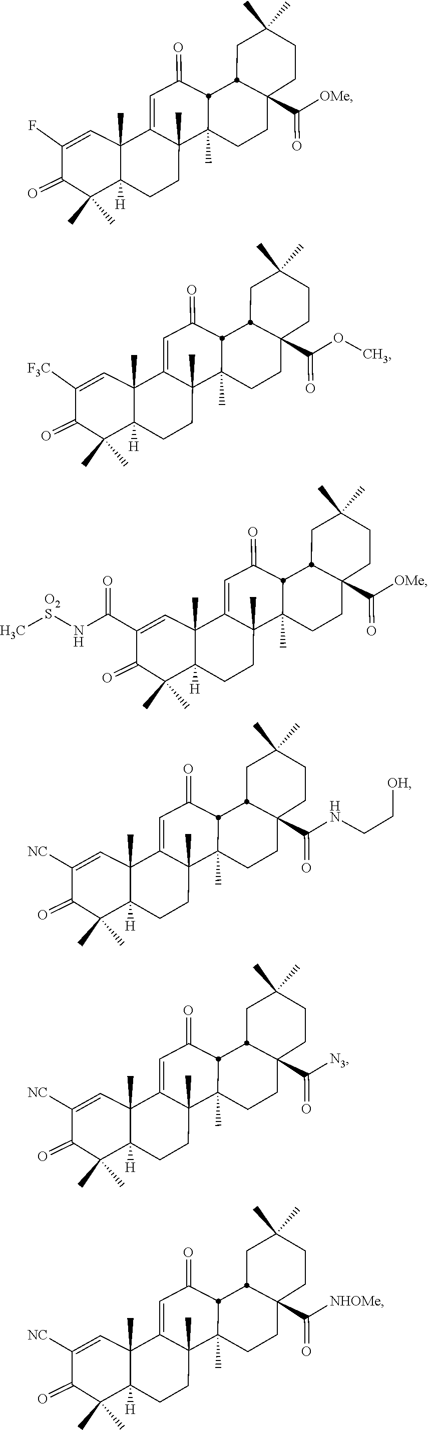

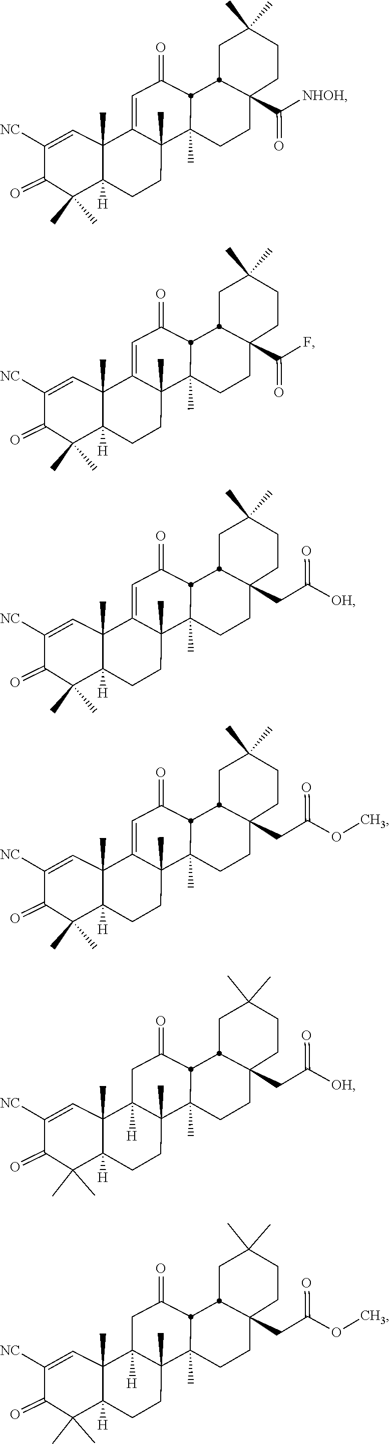

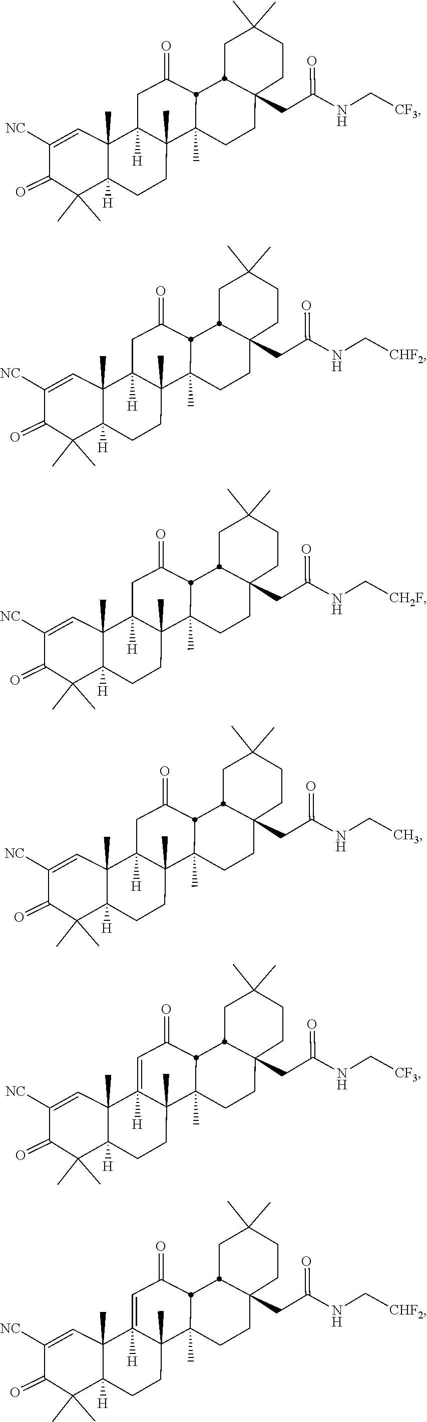

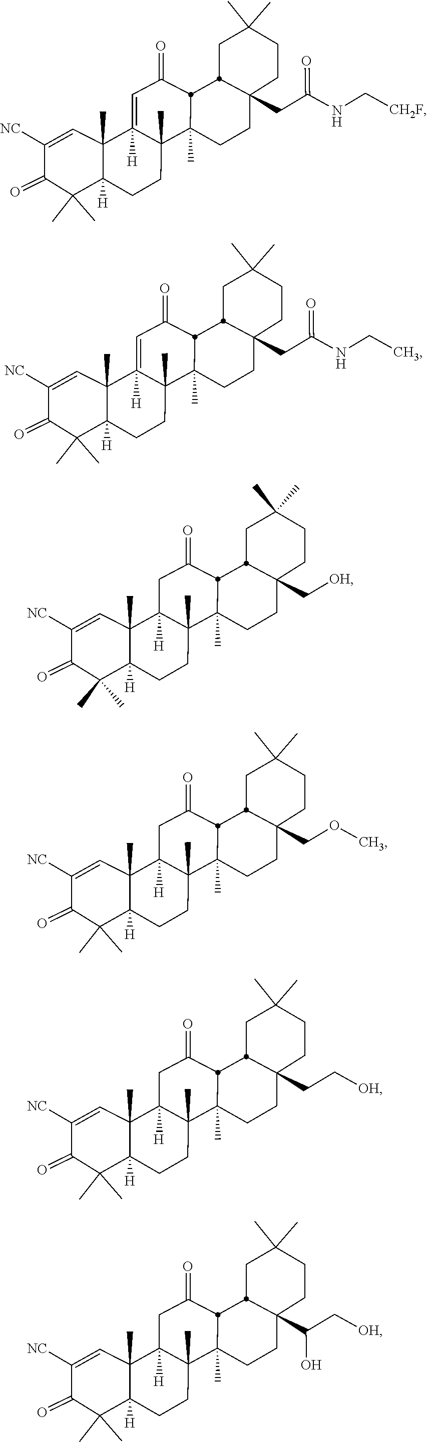

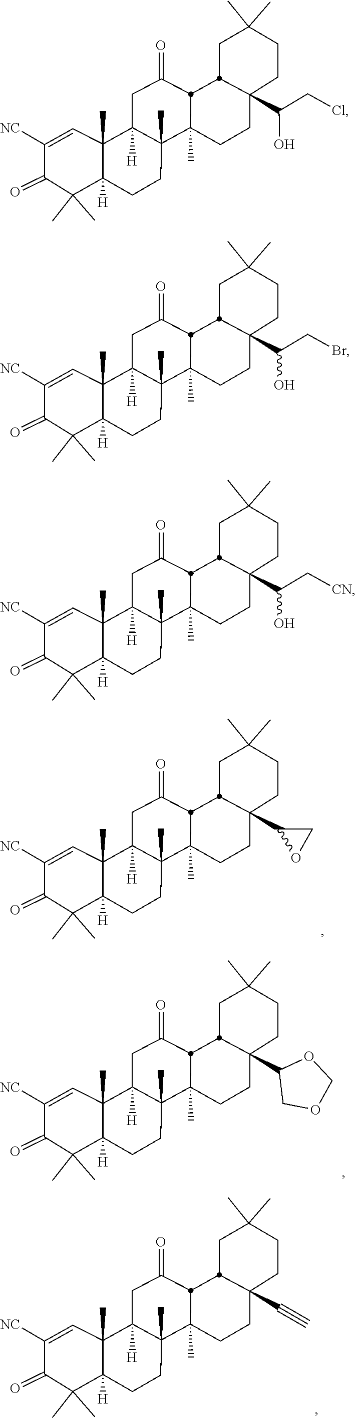

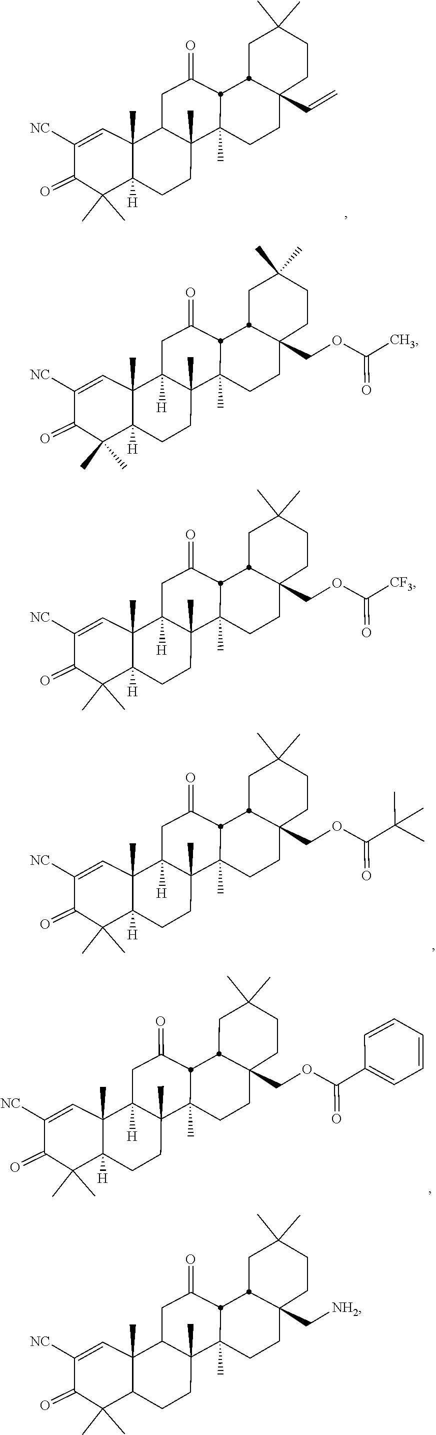

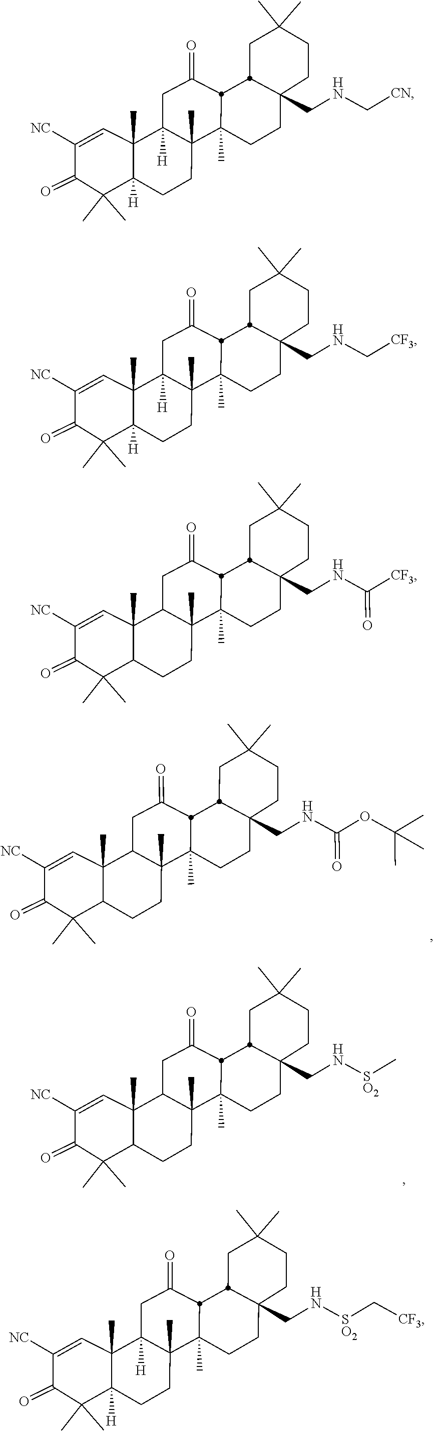

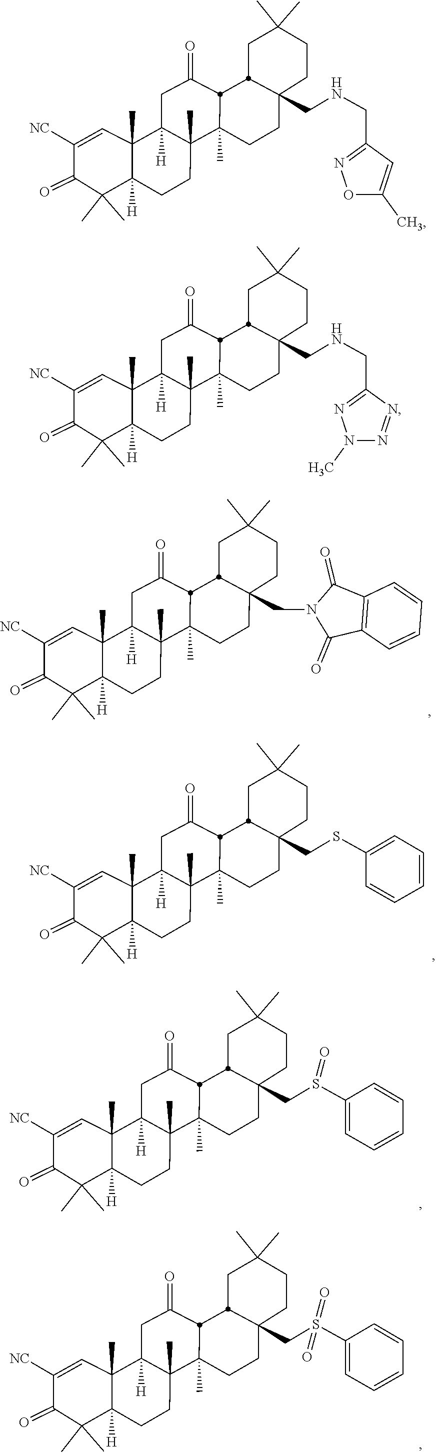

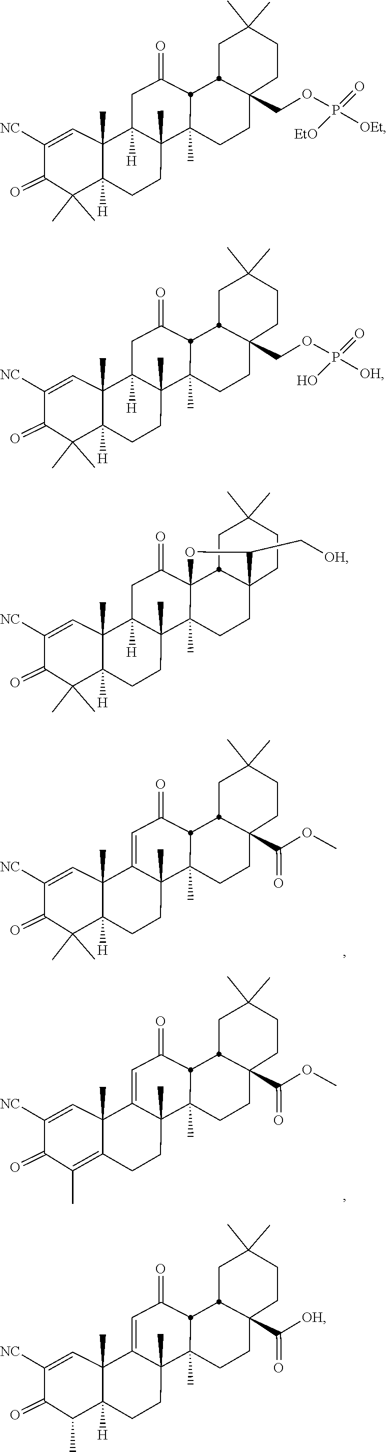

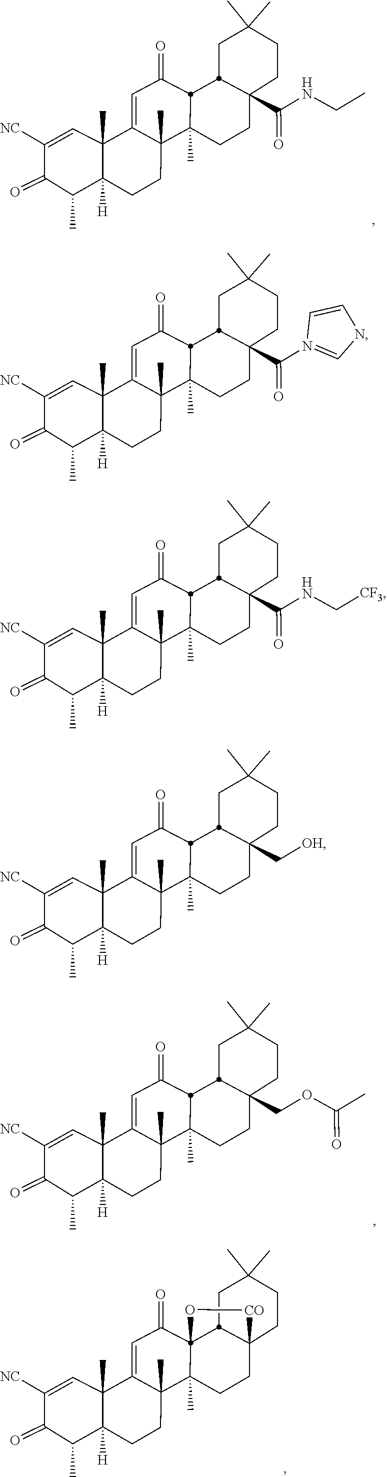

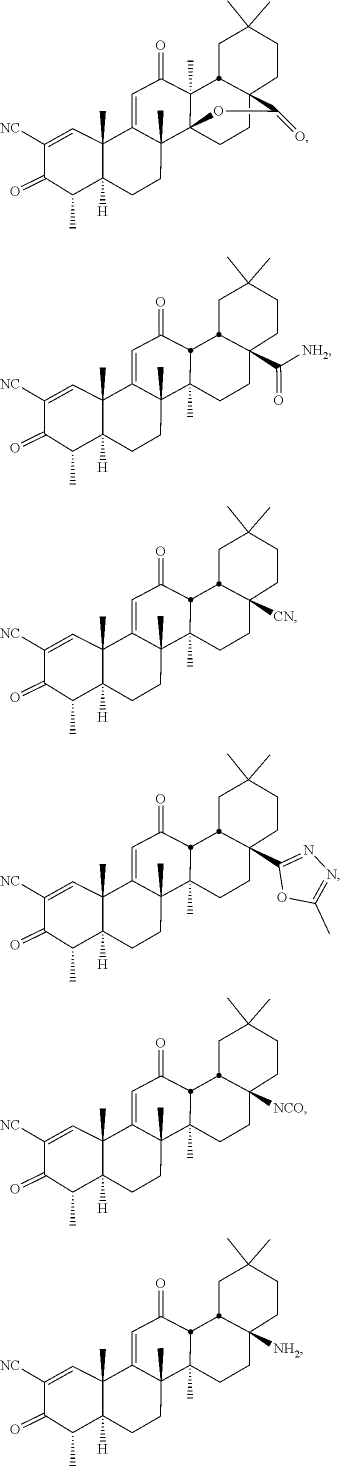

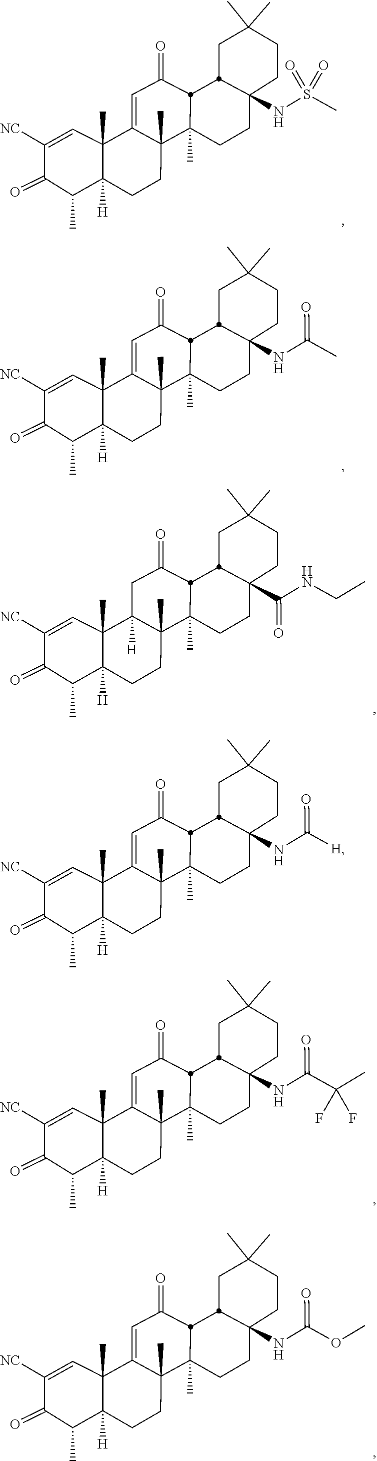

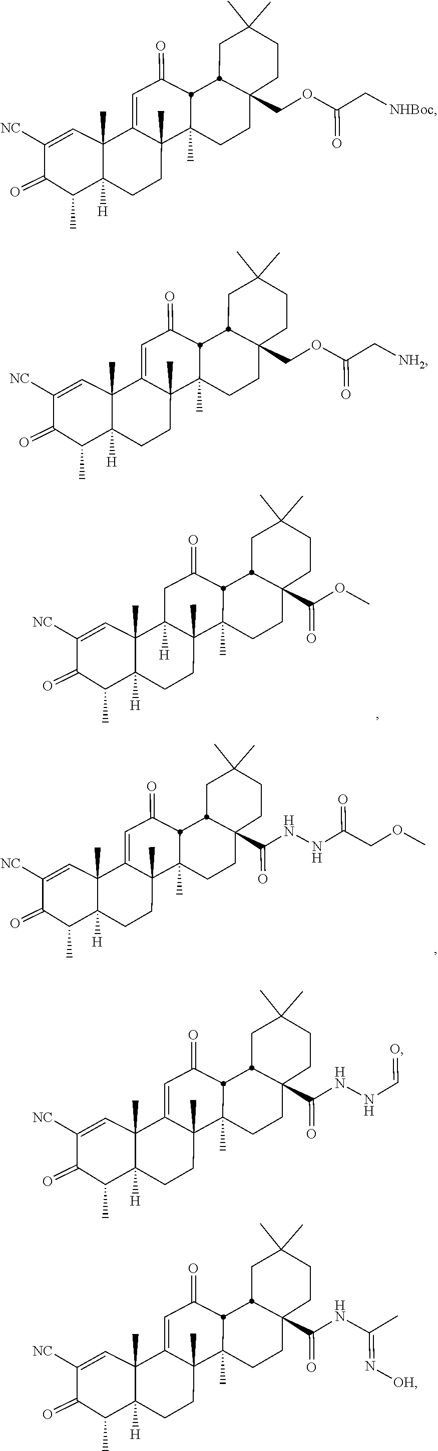

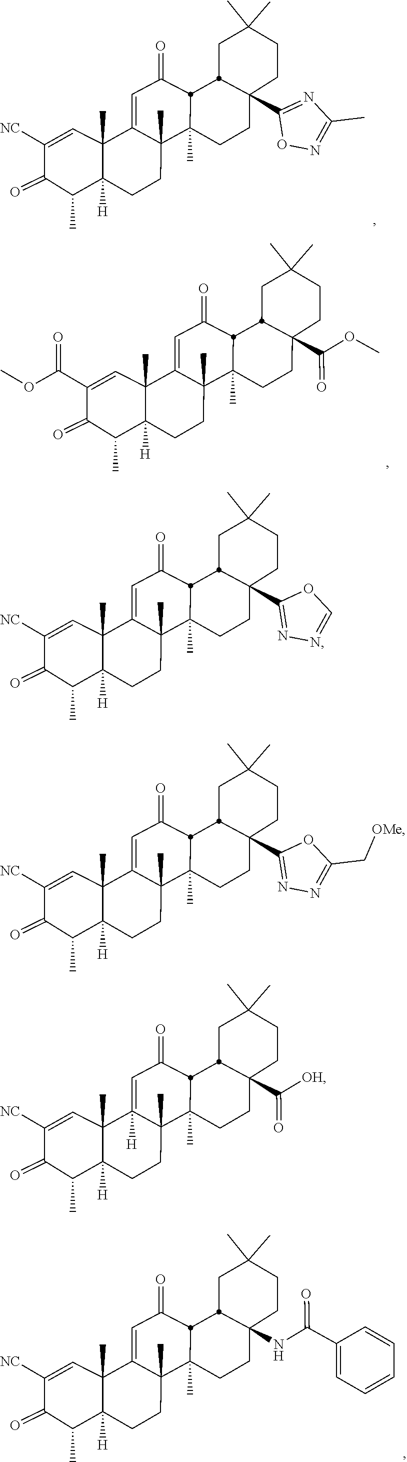

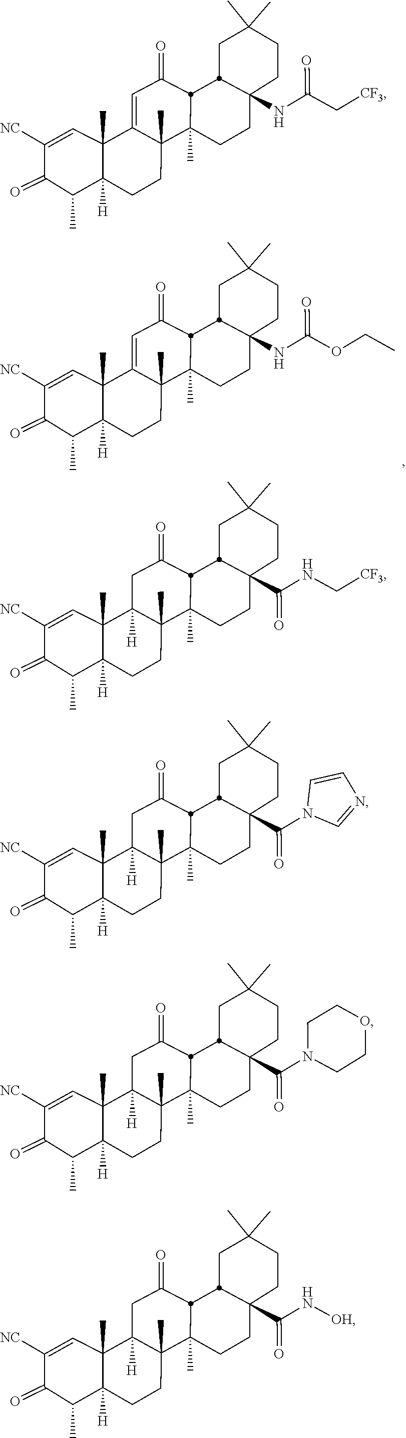

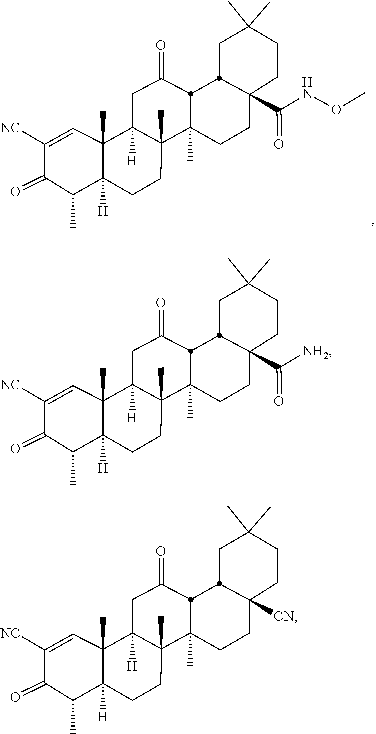

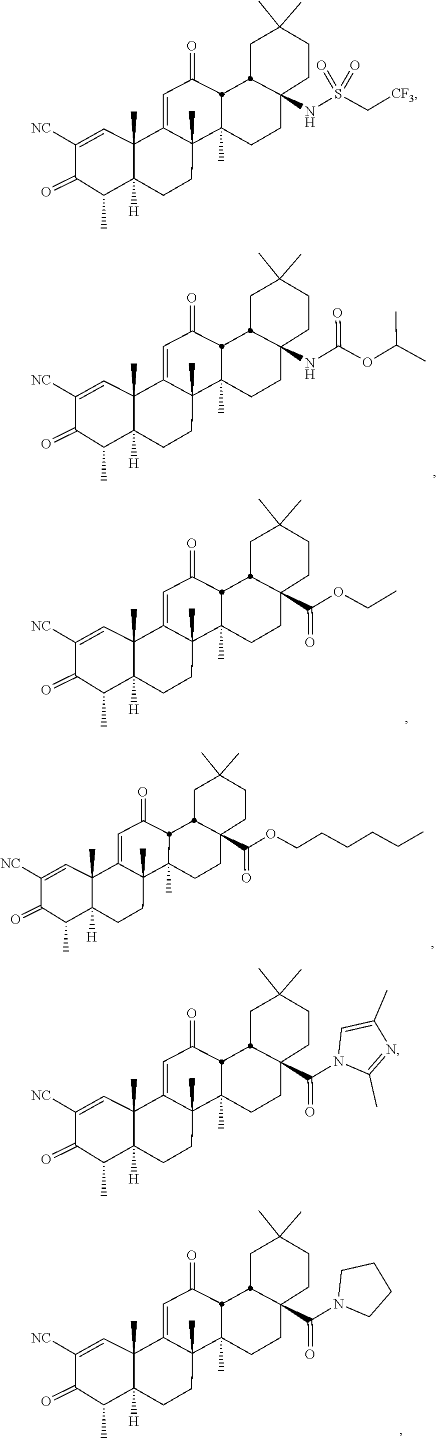

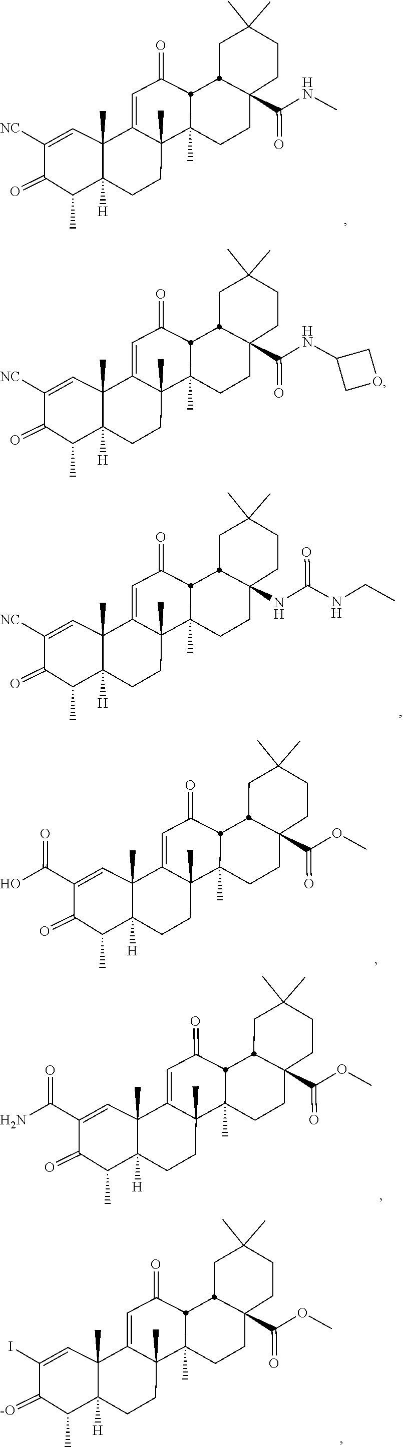

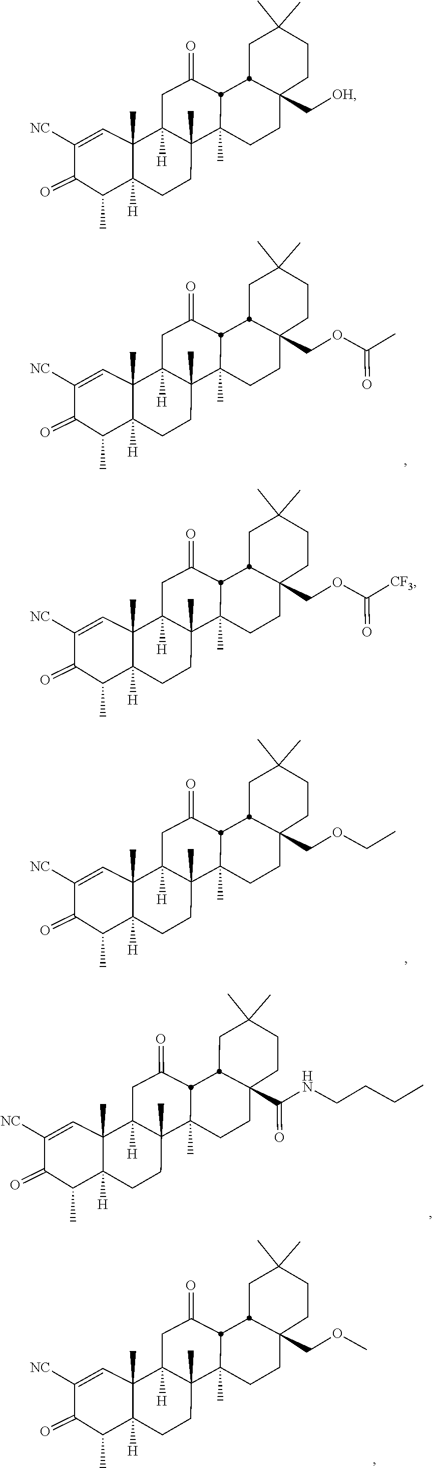

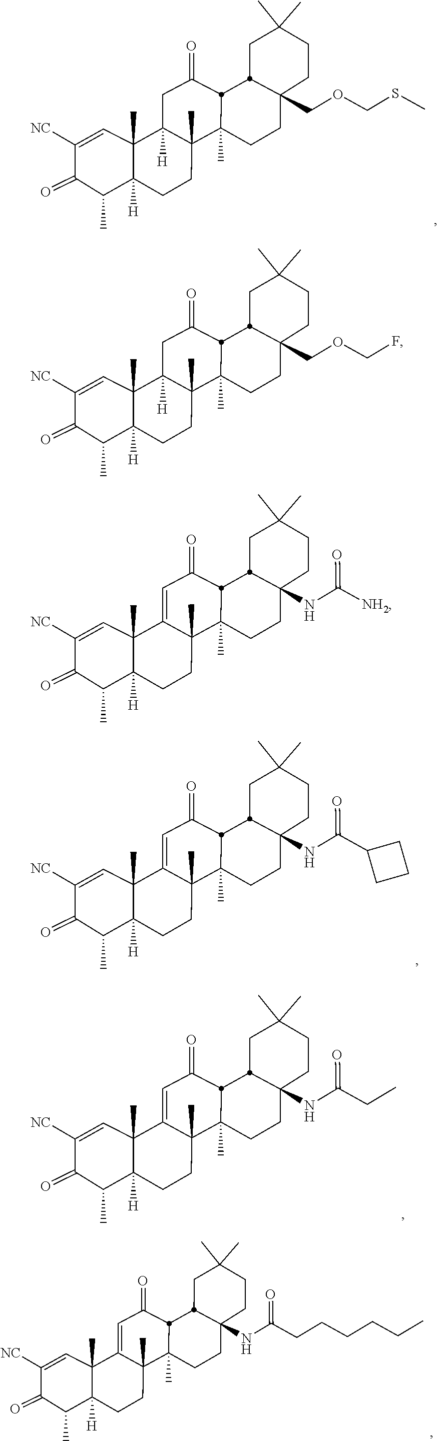

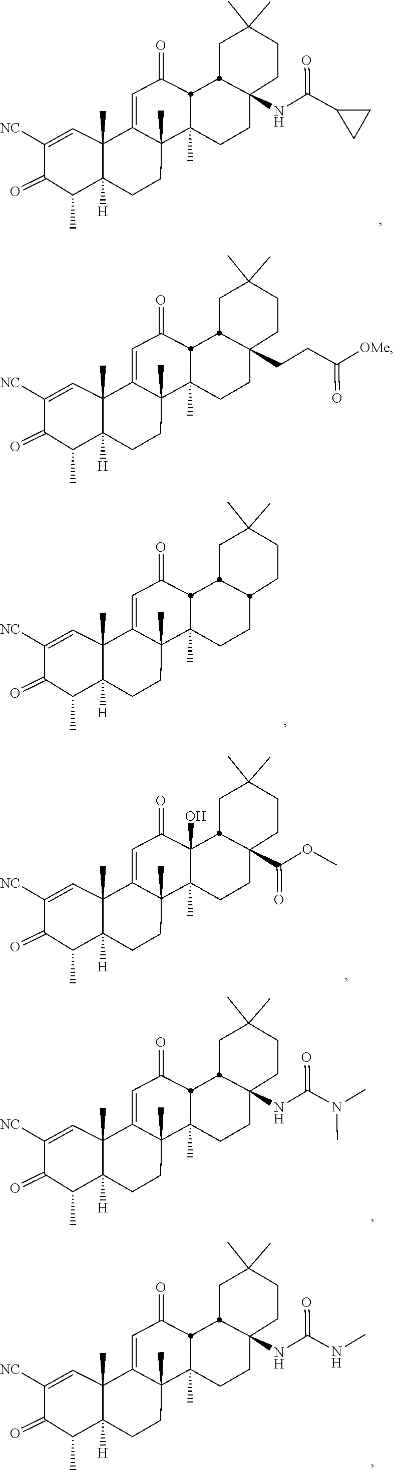

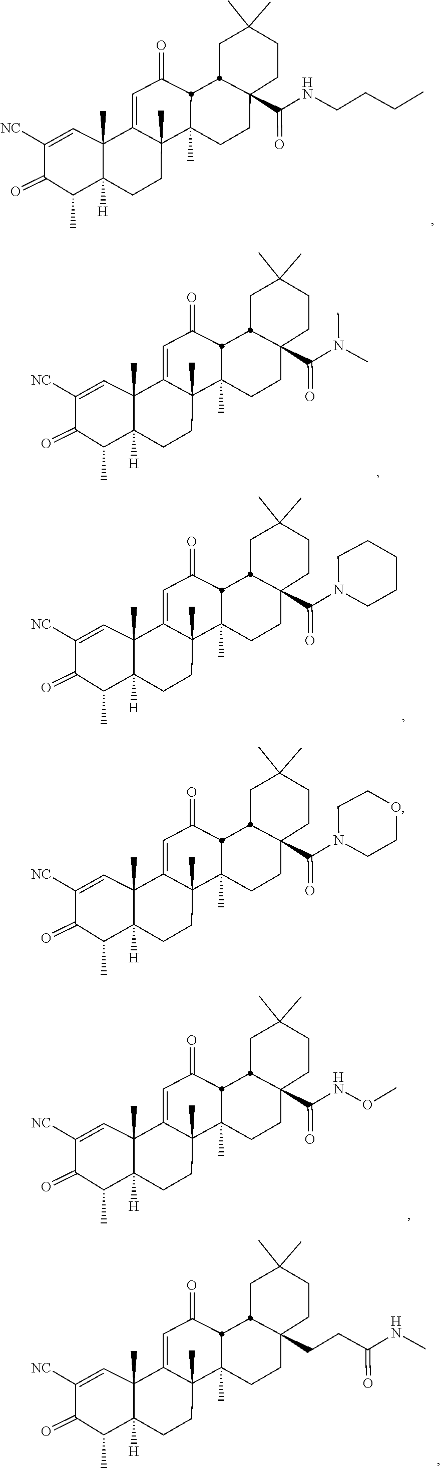

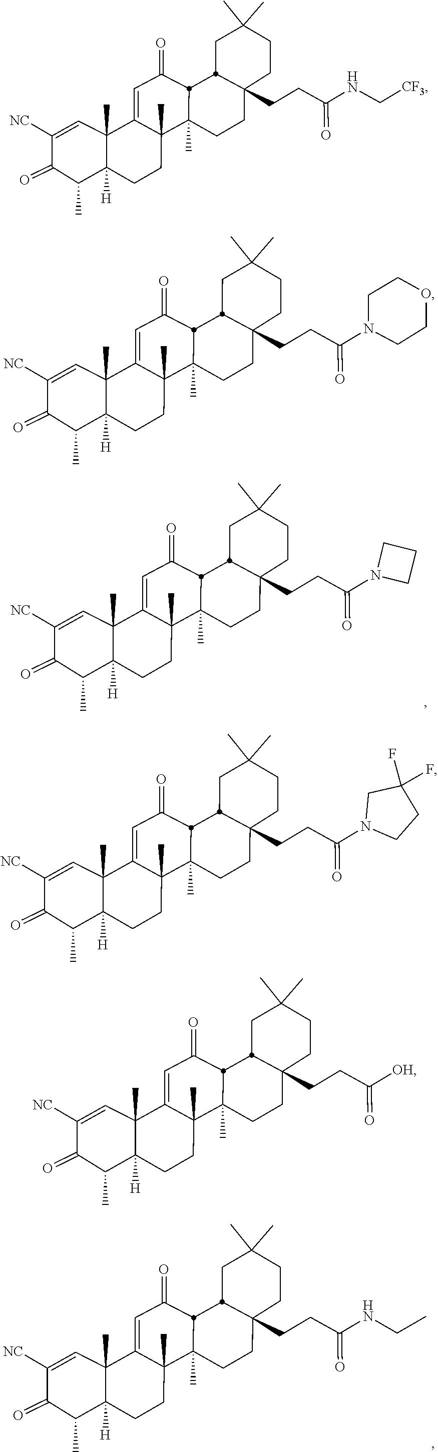

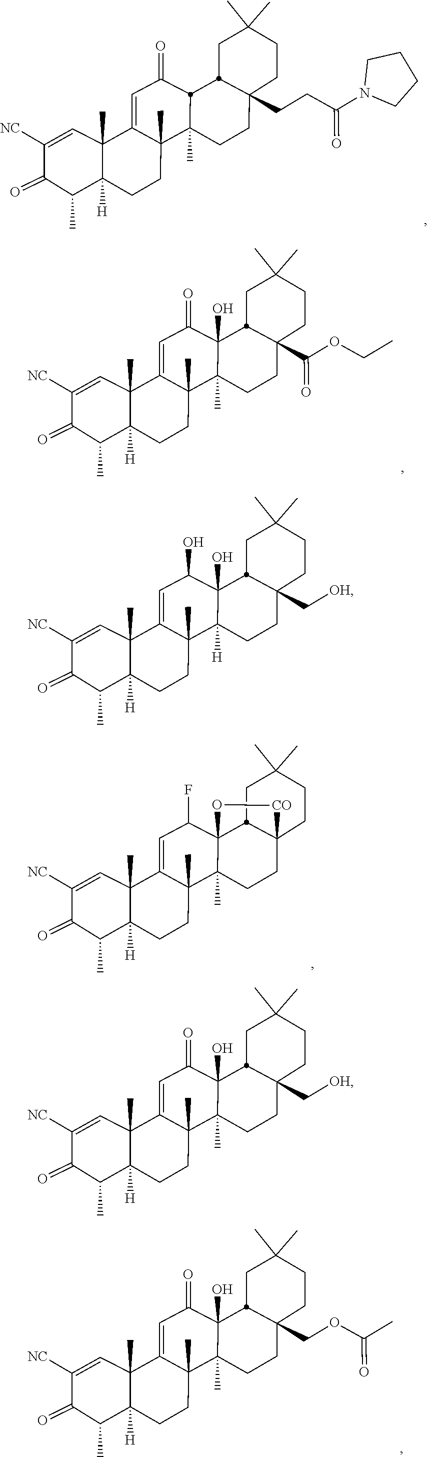

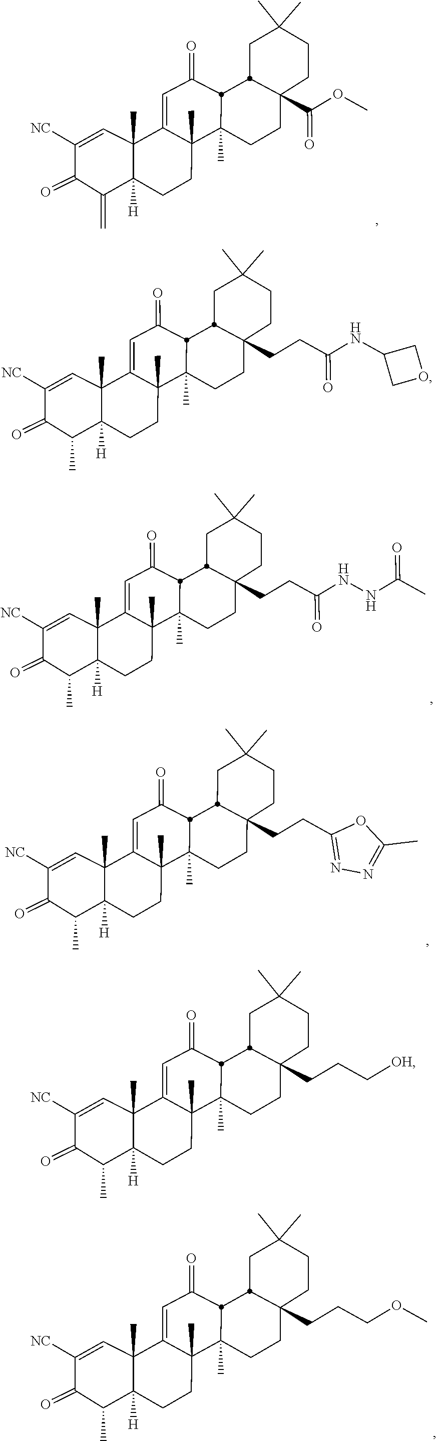

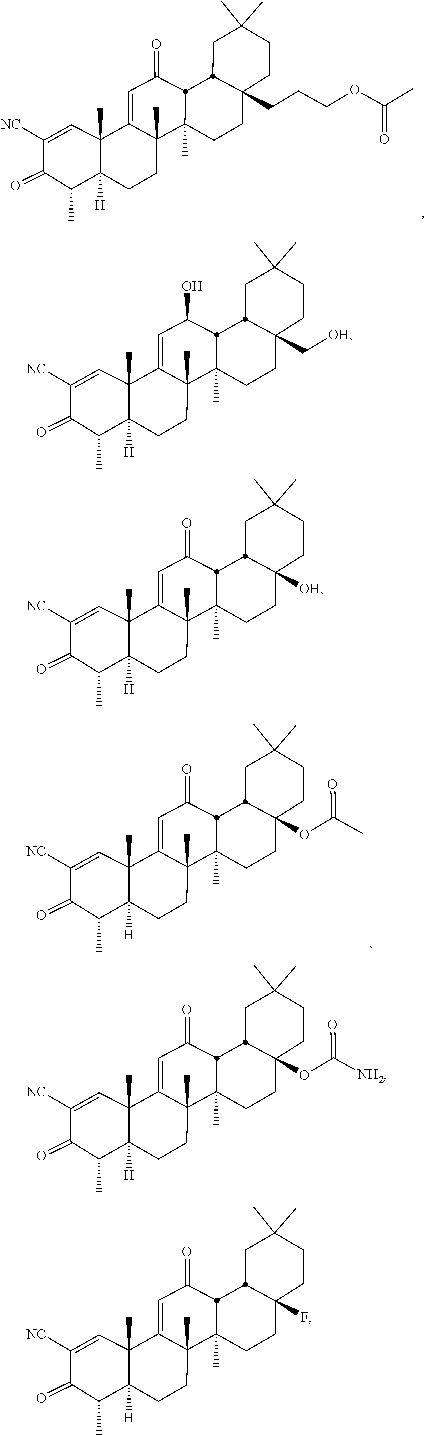

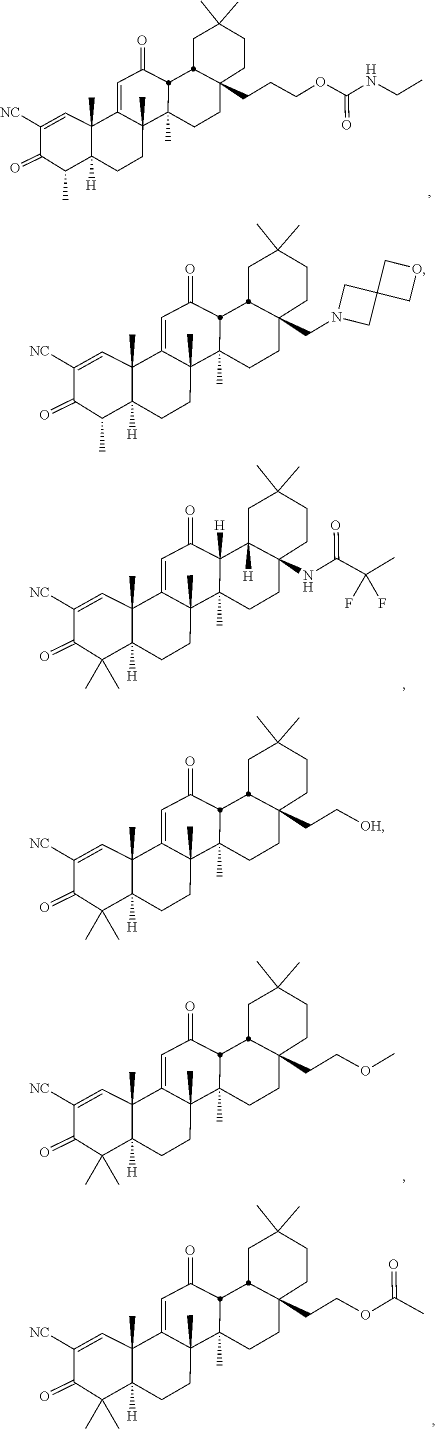

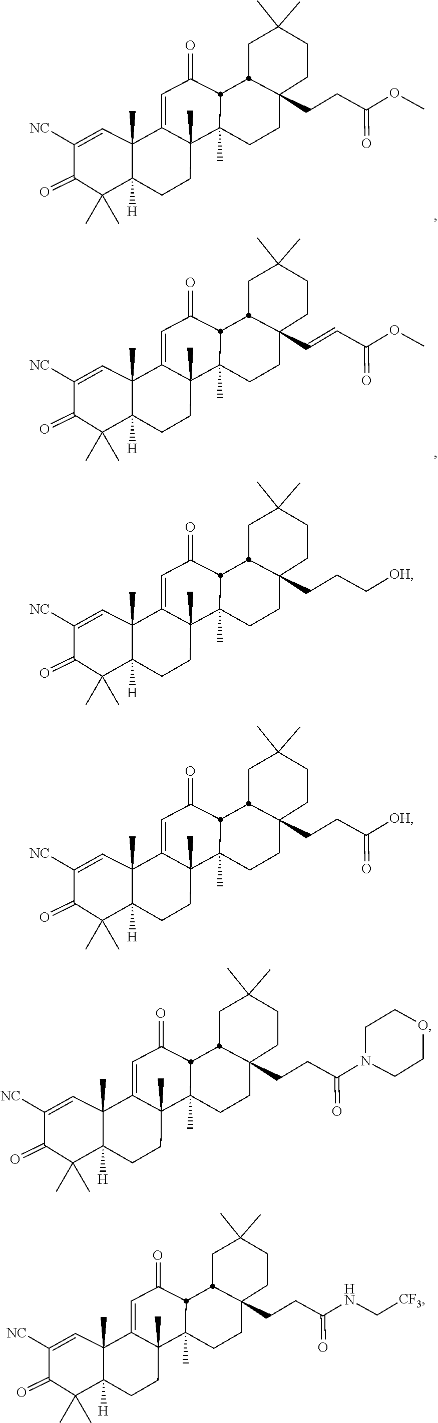

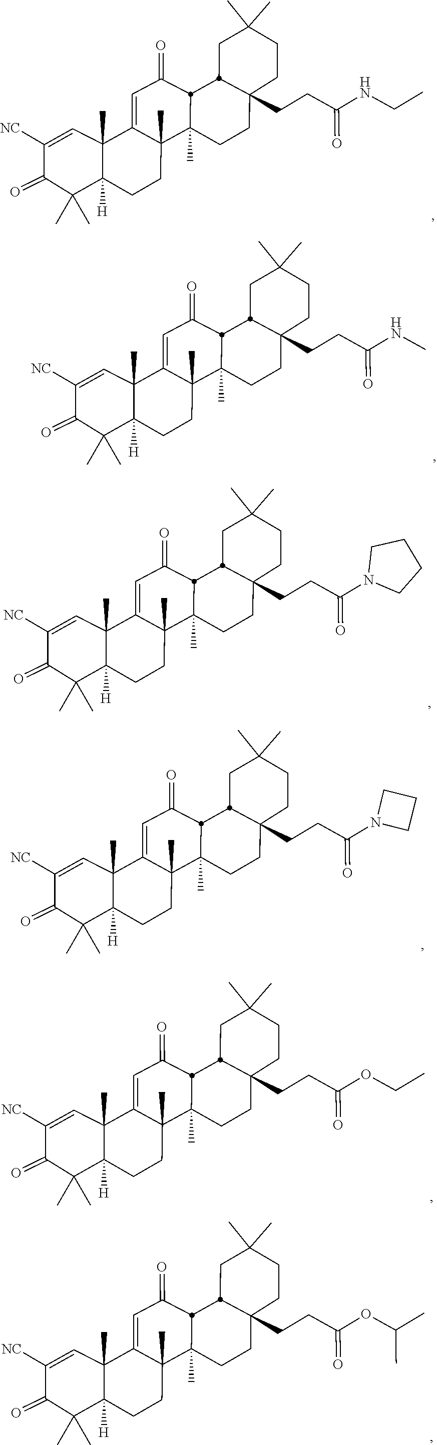

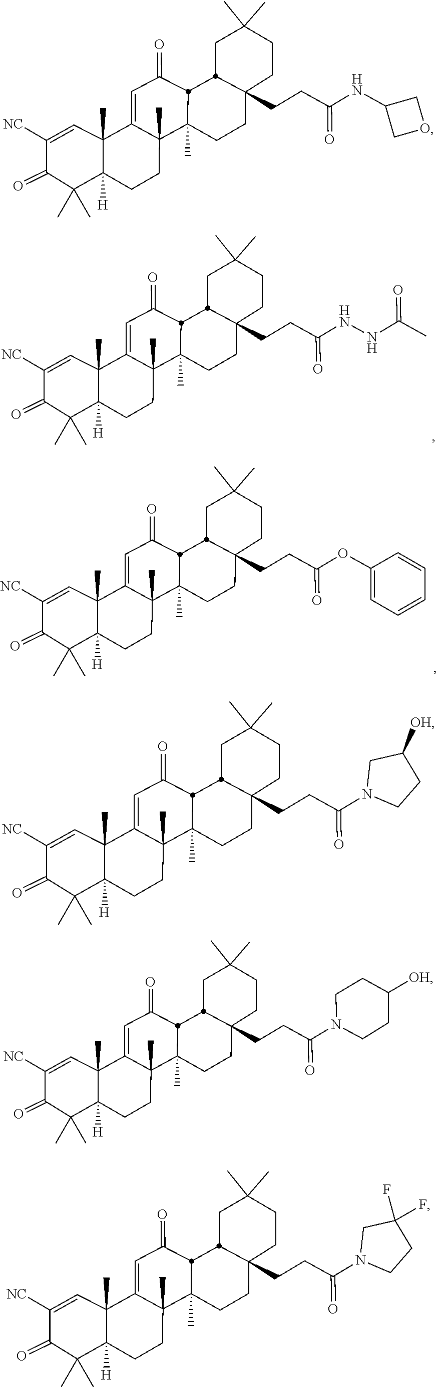

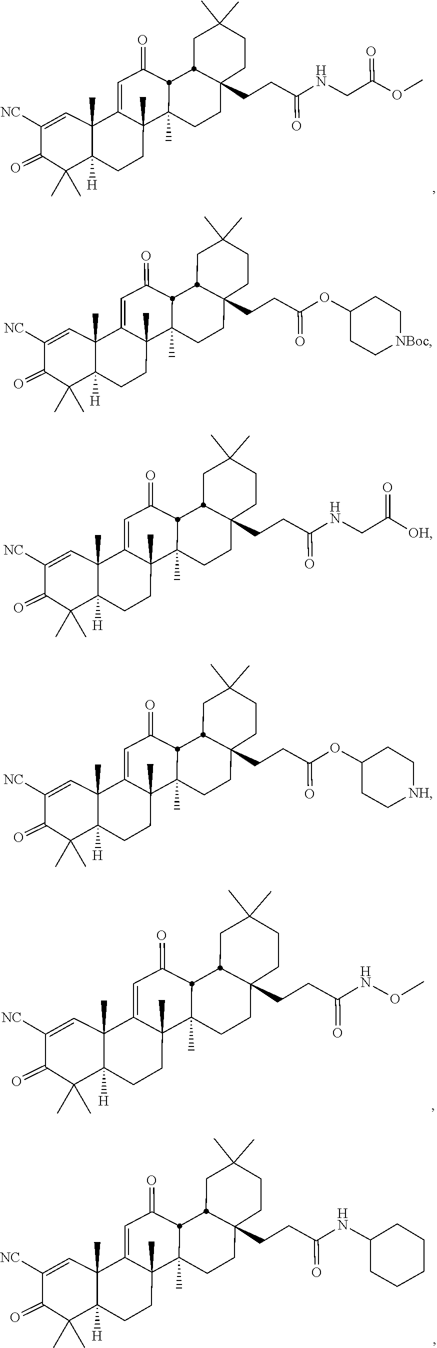

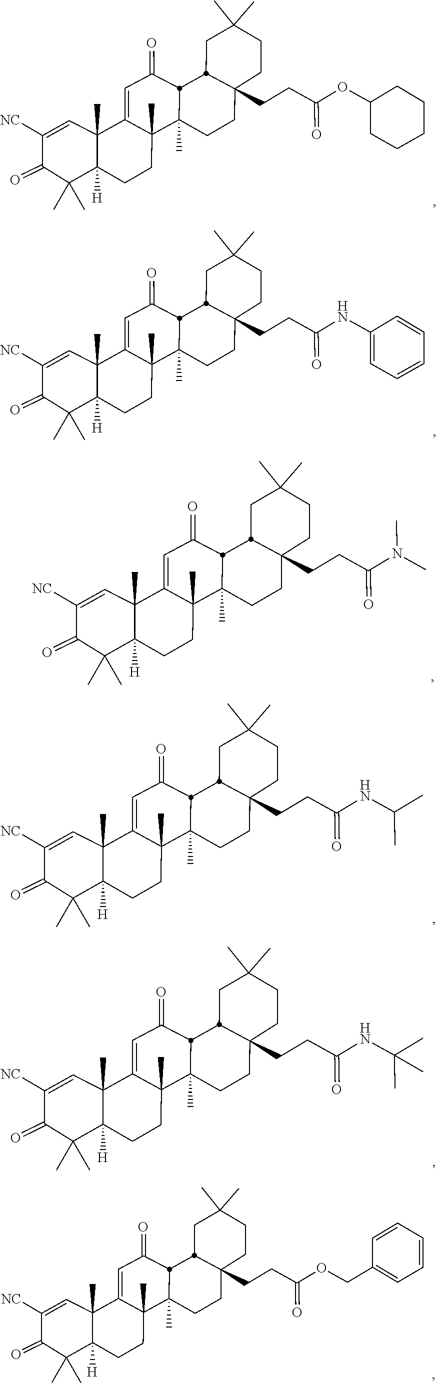

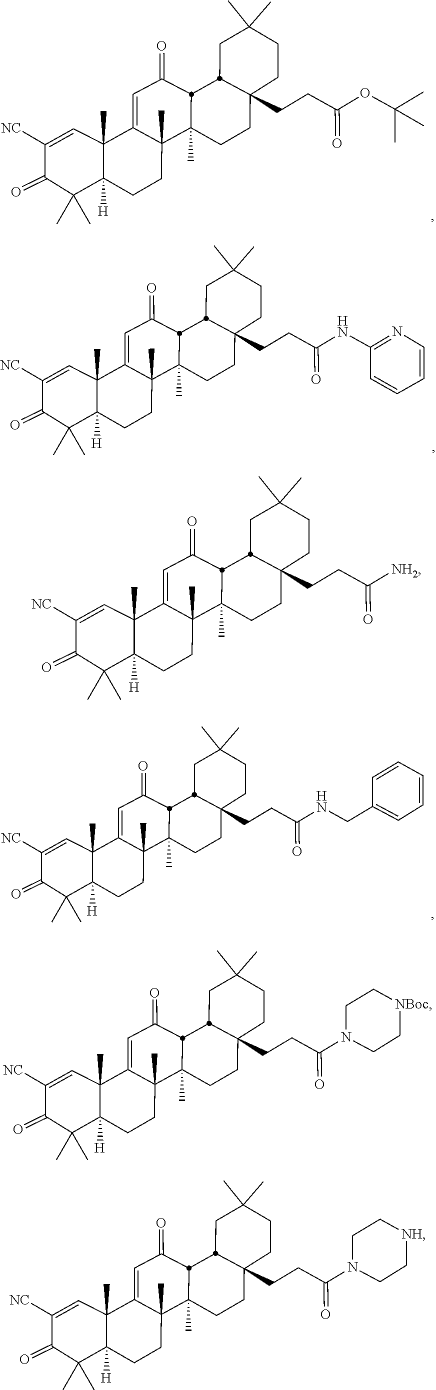

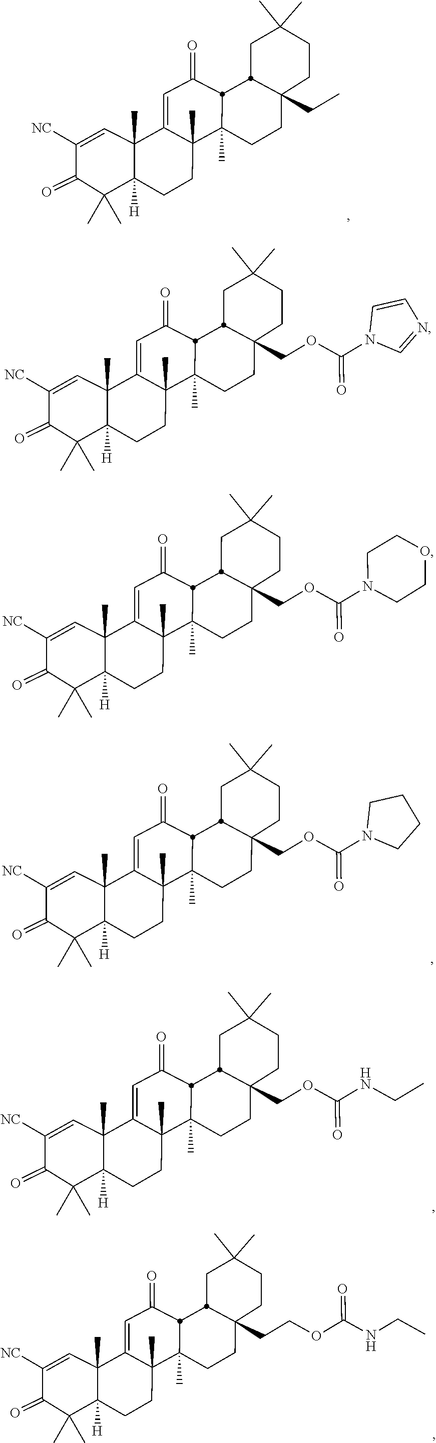

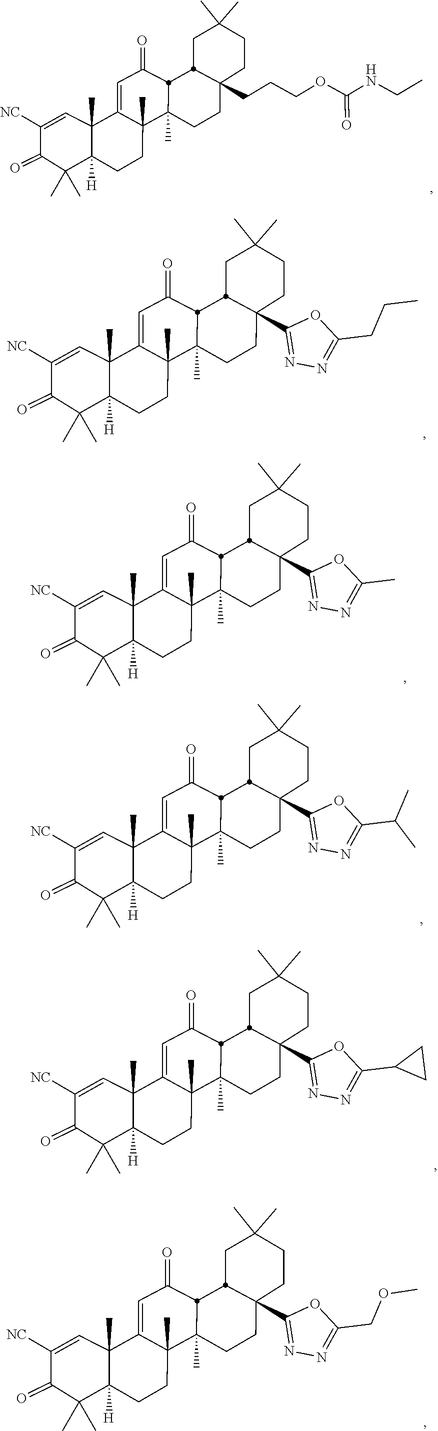

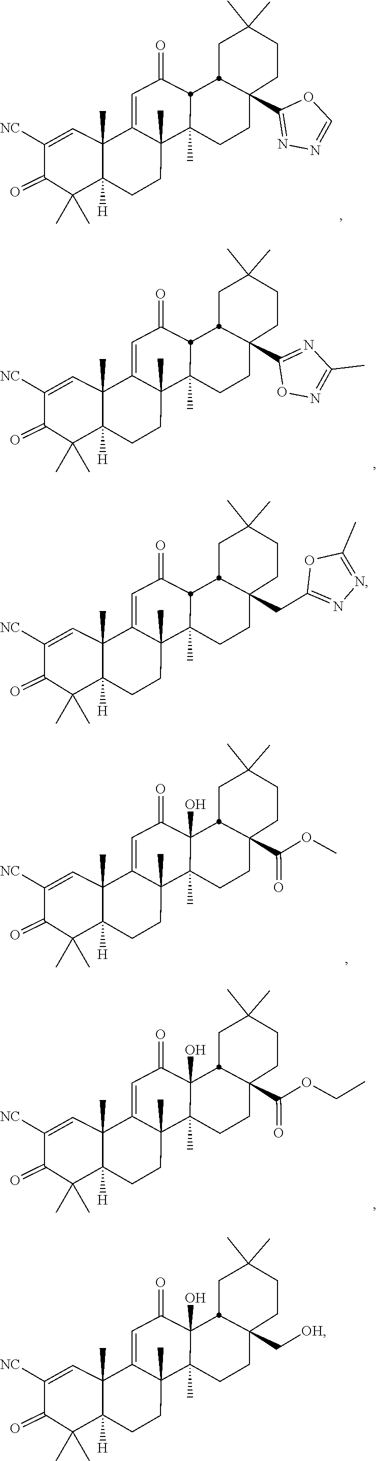

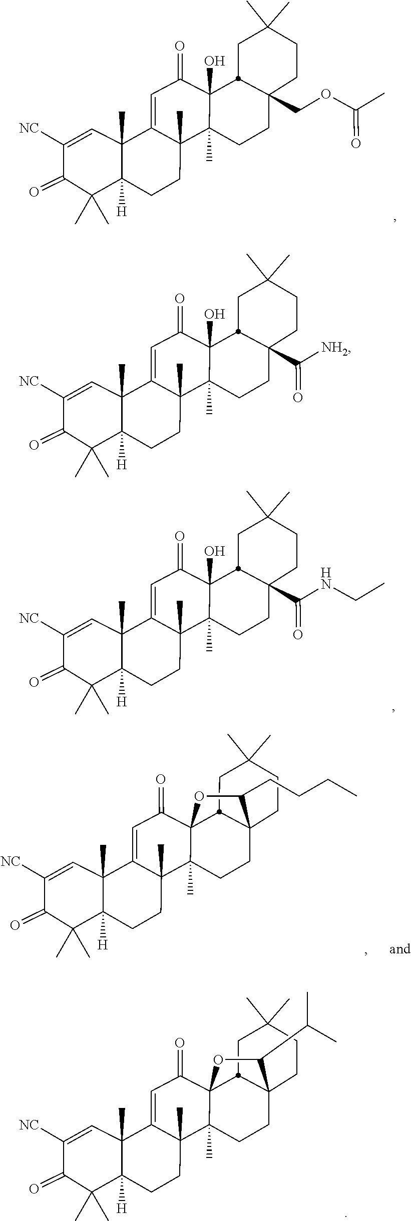

Table 3 summarizes in vitro results for several of these compounds in which RAW264.7 macrophages were pre-treated with DMSO or drugs at various concentrations (nM) for 2 hours, and then treated with 20 ng/mL IFNγ for 24 hours. NO concentration in the media was determined using a Griess reagent system; cell viability was determined using WST-1 reagent. NQO1 CD represents the concentration required to induce a two-fold increase in the expression of NQO1, an Nrf2-regulated antioxidant enzyme, in Hepa1c1c7 murine hepatoma cells (Dinkova-Kostova et al., 2005). All these results are orders of magnitude more active than, for example, the parent oleanolic acid molecule. In part because induction of antioxidant pathways resulting from Nrf2 activation provides important protective effects against oxidative stress and inflammation, analogs of RTA 402 may therefore also be used to for the treatment and/or COVID-19 or symptoms or complications thereof or prevent the onset of symptoms of COVID-19.

TABLE 3
Suppression of IFNγ-induced NO Production

| | RAW264.7 (20 ng/ml IFNγ) | | | | Hepa1c1c7 cells |
|---|---|---|---|---|---|
| Working ID | NO IC₅₀ | | WST-1 IC₅₀ | | NQO1 CD |
| RTA 401 | ~10 | nM | > 200 | nM | 2.3 nM |
| RTA 402 | 2.2 | nM | 80 | nM | 1.0 nM |
| RTA 403 | ~0.6 | nM | 100 | nM | 3.3 nM |
| RTA 404 | 5.8 | nM | 100 | nM | n/a |
| RTA 405 | 6 | nM | ~200 | nM | n/a |
| TP-225 | ~0.4 | nM | 75 | nM | 0.28 nM |

Without being bound by theory, the potency of the compounds of the present invention, e.g., RTA 402, is largely derived from the addition of α,β-unsaturated carbonyl groups. In in vitro assays, most activity of the compounds can be abrogated by the introduction of dithiothreitol (DTT), N-acetyl cysteine (NAC), or glutathione (GSH); thiol containing moieties that interact with α,β-unsaturated carbonyl groups (Wang et al., 2000; Ikeda et al., 2003; 2004; Shishodia et al., 2006). Biochemical assays have established that RTA 402 directly interacts with a critical cysteine residue (C179) on IKKβ (see below) and inhibits its activity (Shishodia et al., 2006; Ahmad et al., 2006). IKKβ controls activation of NF-κB through the “classical” pathway which involves phosphorylation-induced degradation of IκB resulting in release of NF-κB dimers to the nucleus. In macrophages, this pathway is responsible for the production of many pro-inflammatory molecules in response to TNFα and other pro-inflammatory stimuli.

RTA 402 also inhibits the JAK/STAT signaling pathway at multiple levels. JAK proteins are recruited to transmembrane receptors (e.g., IL-6R) upon activation by ligands such as interferons and interleukins. JAKs then phosphorylate the intracellular portion of the receptor causing recruitment of STAT transcription factors. The STATs are then phosphorylated by JAKs, form dimers, and translocate to the nucleus where they activate transcription of several genes involved in inflammation. RTA 402 inhibits constitutive and IL-6-induced STAT3 phosphorylation and dimer formation and directly binds to cysteine residues in STAT3 (C259) and in the kinase domain of JAK1 (C1077). Biochemical assays have also established that the triterpenoids directly interact with critical cysteine residues on Keap1 (Dinkova-Kostova et al., 2005). Keap1 is an actin-tethered protein that keeps the transcription factor Nrf2 sequestered in the cytoplasm under normal conditions (Kobayashi and Yamamoto, 2005). Oxidative stress results in oxidation of the regulatory cysteine residues on Keap1 and causes the release of Nrf2. Nrf2 then translocates to the nucleus and binds to antioxidant response elements (AREs) resulting in transcriptional activation of many antioxidant and anti-inflammatory genes. Another target of the Keap1/Nrf2/ARE pathway is heme oxygenase 1 (HO-1). HO-1 breaks down heme into bilirubin and carbon monoxide and plays many antioxidant and anti-inflammatory roles (Maines and Gibbs, 2005). HO-1 has recently been shown to be potently induced by the triterpenoids (Liby et al., 2005), including RTA 402. RTA 402 and many structural analogs have also been shown to be potent inducers of the expression of other Phase 2 proteins (Yates et al., 2007). RTA 402 is a potent inhibitor of NF-κB activation. Furthermore, RTA 402 activates the Keap1/Nrf2/ARE pathway and induces expression of HO-1.

Compounds employed may be made using the methods described by Honda et al. (2000a); Honda et al. (2000b); Honda et al. (2002); and U.S. Patent Application Publications 2009/0326063, 2010/0056777, 2010/0048892, 2010/0048911, 2010/0041904, 2003/0232786, 2008/0261985 and 2010/0048887, all of which are incorporated by reference herein. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure (2013), which is incorporated by reference herein. In addition, the synthetic methods may be further modified and optimized for preparative, pilot- or large-scale production, either batch of continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

Compounds of the present invention may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration.

Chemical formulas used to represent compounds of the present invention will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

Polymorphic forms of the compounds of the present invention, e.g., Forms A, B, C, D, and I-VI of CDDO-Me, may be used in accordance with the methods of this inventions. Form B displays a bioavailability that is surprisingly better than that of Form A. Specifically, the bioavailability of Form B was higher than that of Form A CDDO-Me in monkeys when the monkeys received equivalent dosages of the two forms orally, in gelatin capsules. See U.S. Patent Application Publication 2009/0048204, PCT Publication WO 2019014412, Chinese Patent Publication CN 102887936, and Chinese Patent Publication CN 102875634, each of which is incorporated by reference herein in its entirety.

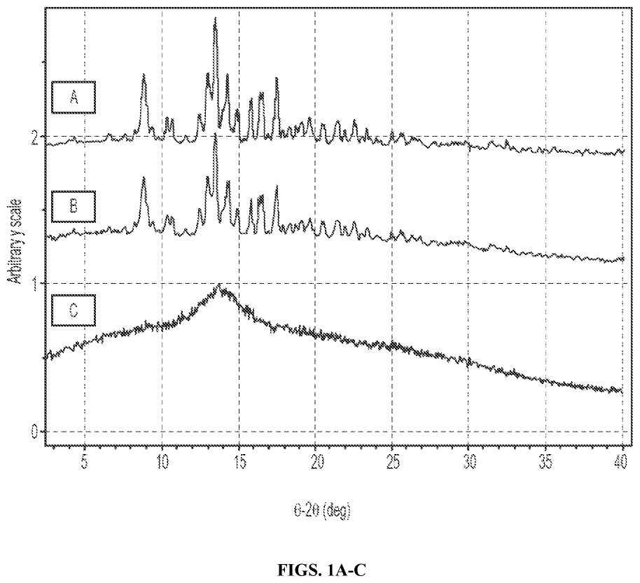

“Form A” of CDDO-Me (RTA 402) is unsolvated (non-hydrous) and can be characterized by a distinctive crystal structure, with a space group of P4₃ 2₁2 (no. 96) unit cell dimensions of a=14.2 Å, b=14.2 Å and c=81.6 Å, and by a packing structure, whereby three molecules are packed in helical fashion down the crystallographic b axis. In some embodiments, Form A can also be characterized by X-ray powder diffraction (XRPD) pattern (CuKα) comprising significant diffraction peaks at about 8.8, 12.9, 13.4, 14.2 and 17.4°θ. In some variations, the X-ray powder diffraction of Form A is substantially as shown in FIG. 1A or FIG. 1B.

Unlike Form A, “Form B” of CDDO-Me is in a single phase but lacks such a defined crystal structure. Samples of Form B show no long-range molecular correlation, i.e., above roughly 20 Å. Moreover, thermal analysis of Form B samples reveals a glass transition temperature (T_{g}) in a range from about 120° C. to about 130° C. In contrast, a disordered nanocrystalline material does not display a T_{g} but instead only a melting temperature (Tₘ), above which crystalline structure becomes a liquid. Form B is typified by an XRPD spectrum (FIG. 1C) differing from that of Form A (FIG. 1A or FIG. 1B). Since it does not have a defined crystal structure, Form B likewise lacks distinct XRPD peaks, such as those that typify Form A, and instead is characterized by a general “halo” XRPD pattern. In particular, the non-crystalline Form B falls into the category of “X-ray amorphous” solids because its XRPD pattern exhibits three or fewer primary diffraction halos. Within this category, Form B is a “glassy” material.

Form A and Form B of CDDO-Me are readily prepared from a variety of solutions of the compound. For example, Form B can be prepared by fast evaporation or slow evaporation in MTBE, THF, toluene, or ethyl acetate. Form A can be prepared in several ways, including via fast evaporation, slow evaporation, or slow cooling of a CDDO-Me solution in ethanol or methanol. Preparations of CDDO-Me in acetone can produce either Form A, using fast evaporation, or Form B, using slow evaporation.

Various means of characterization can be used together to distinguish Form A and Form B CDDO-Me from each other and from other forms of CDDO-Me. Illustrative of the techniques suitable for this purpose are solid state Nuclear Magnetic Resonance (NMR), X-ray powder diffraction (compare FIGS. 1A & B with FIG. 1C), X-ray crystallography, differential scanning calorimetry (DSC), dynamic vapor sorption/desorption (DVS), Karl Fischer analysis (KF), hot stage microscopy, modulated differential screening calorimetry, FT-IR, and Raman spectroscopy. In particular, analysis of the XRPD and DSC data can distinguish Form A, Form B, and a hemibenzenate form of CDDO-Me. See U.S. Patent Application Publication 2009/0048204, which is incorporated by reference herein in its entirety.

Additional details regarding polymorphic forms of CDDO-Me are described in U.S. Patent Application Publication 2009/0048204, PCT Publication WO 2009/023232, PCT Publication WO 2010/093944, PCT Publication WO 2019014412, Chinese Patent Publication CN 102887936, and Chinese Patent Publication CN 102875634, which are all incorporated herein by reference in their entireties.

Non-limiting specific formulations of the compounds disclosed herein include CDDO-Me polymer dispersions. See, for example, PCT Publication WO 2010/093944, which is incorporated herein by reference in its entirety. Some of the formulations reported therein exhibit higher bioavailability than either the micronized Form A or nanocrystalline Form A formulations. Additionally, the polymer dispersion-based formulations demonstrate further surprising improvements in oral bioavailability relative to the micronized Form B formulations. For example, the methacrylic acid copolymer, Type C and HPMC-P formulations showed the greatest bioavailability in the subject monkeys.

Compounds employed in methods of the invention may also exist in prodrug form. Since prodrugs enhance numerous desirable qualities of pharmaceuticals, e.g., solubility, bioavailability, manufacturing, etc., the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject or patient, cleaves to form a hydroxy, amino, or carboxylic acid, respectively.

It should be recognized that the particular anion or cation forming a part of any salt of this invention is not critical, so long as the salt, as a whole, is pharmacologically acceptable. Examples of pharmaceutically acceptable salts and their methods of preparation and use are presented in Handbook of Pharmaceutical Salts: Properties, and Use (2002), which is incorporated herein by reference.

In some embodiments, the compounds employed in the methods described in the present invention have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

### III. Pharmaceutical Formulations and Routes of Administration

Administration of the compounds of the present invention to a patient will follow general protocols for the administration of pharmaceuticals, taking into account the toxicity, if any, of the drug. It is expected that the treatment cycles would be repeated as necessary.

The compounds of the present invention may be administered by a variety of methods, e.g., orally or by injection (e.g., subcutaneous, intravenous, intraperitoneal, etc.). Depending on the route of administration, the active compounds may be coated by a material to protect the compound from the action of acids and other natural conditions which may inactivate the compound. They may also be administered by continuous perfusion/infusion of a disease or wound site. Specific examples of formulations, including a polymer-based dispersion of CDDO-Me that showed improved oral bioavailability, are provided in U.S. Patent Application Publication No. 2009/0048204, which is incorporated herein by reference in its entirety. It will be recognized by those skilled in the art that other methods of manufacture may be used to produce dispersions of the present invention with equivalent properties and utility (see, Repka et al., 2002 and references cited therein). Such alternative methods include but are not limited to solvent evaporation, extrusion, such as hot melt extrusion, and other techniques.

To administer the active compound by other than parenteral administration, it may be necessary to coat the compound with, or co-administer the compound with, a material to prevent its inactivation. For example, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The therapeutic compound may also be administered parenterally, intraperitoneally, intraspinally, or intracerebrally. Dispersions may be prepared in, e.g., glycerol, liquid polyethylene glycols, mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

The therapeutic compound can be orally administered, for example, with an inert diluent or an assimilable edible carrier. The therapeutic compound and other ingredients may also be enclosed in a hard- or soft-shell gelatin capsule, compressed into tablets, or incorporated directly into the subject's or patient's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects or patients to be treated; each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

The therapeutic compound may also be administered topically to the skin, eye, or mucosa. Alternatively, if local delivery to the lungs is desired the therapeutic compound may be administered by inhalation in a dry-powder or aerosol formulation.

The therapeutic compound may be formulated in a biocompatible matrix for use in a drug-eluting stent.

In some embodiments, the effective dose range for the therapeutic compound can be extrapolated from effective doses determined in animal studies for a variety of different animals. In general a human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present invention or composition comprising a compound of the present invention administered to a subject or a patient may be determined by physical and physiological factors such as age, sex, body weight, severity of condition, the type of disease being treated, previous or concurrent therapeutic interventions, idiopathy of the subject or the patient and on the route of administration. These factors may be determined by a skilled artisan. The practitioner responsible for administration will typically determine the concentration of active ingredient(s) in a composition and appropriate dose(s) for the individual subject or patient. The dosage may be adjusted by the individual physician in the event of any complication.

In some embodiments, the pharmaceutically effective amount is a daily dose from about 0.1 mg to about 500 mg of the compound. In some variations, the daily dose is from about 1 mg to about 300 mg of the compound. In some variations, the daily dose is from about 10 mg to about 200 mg of the compound. In some variations, the daily dose is about 25 mg of the compound. In other variations, the daily dose is about 75 mg of the compound. In still other variations, the daily dose is about 150 mg of the compound. In further variations, the daily dose is from about 0.1 mg to about 30 mg of the compound. In some variations, the daily dose is from about 0.5 mg to about 20 mg of the compound. In some variations, the daily dose is from about 1 mg to about 15 mg of the compound. In some variations, the daily dose is from about 1 mg to about 10 mg of the compound. In some variations, the daily dose is from about 1 mg to about 5 mg of the compound.

In some embodiments, the pharmaceutically effective amount is a daily dose of 0.01-25 mg of compound per kg of body weight. In some variations, the daily dose is 0.05-20 mg of compound per kg of body weight. In some variations, the daily dose is 0.1-10 mg of compound per kg of body weight. In some variations, the daily dose is 0.1-5 mg of compound per kg of body weight. In some variations, the daily dose is 0.1-2.5 mg of compound per kg of body weight.

In some embodiments, the pharmaceutically effective amount is a daily dose of 0.1-1000 mg of compound per kg of body weight. In some variations, the daily dose is 0.15-20 mg of compound per kg of body weight. In some variations, the daily dose is 0.20-10 mg of compound per kg of body weight. In some variations, the daily dose is 0.40-3 mg of compound per kg of body weight. In some variations, the daily dose is 0.50-9 mg of compound per kg of body weight. In some variations, the daily dose is 0.60-8 mg of compound per kg of body weight. In some variations, the daily dose is 0.70-7 mg of compound per kg of body weight. In some variations, the daily dose is 0.80-6 mg of compound per kg of body weight. In some variations, the daily dose is 0.90-5 mg of compound per kg of body weight. In some variations, the daily dose is from about 1 mg to about 5 mg of compound per kg of body weight.

An effective amount typically will vary from about 0.001 mg/kg to about 1,000 mg/kg, from about 0.01 mg/kg to about 750 mg/kg, from about 0.1 mg/kg to about 500 mg/kg, from about 0.2 mg/kg to about 250 mg/kg, from about 0.3 mg/kg to about 150 mg/kg, from about 0.3 mg/kg to about 100 mg/kg, from about 0.4 mg/kg to about 75 mg/kg, from about 0.5 mg/kg to about 50 mg/kg, from about 0.6 mg/kg to about 30 mg/kg, from about 0.7 mg/kg to about 25 mg/kg, from about 0.8 mg/kg to about 15 mg/kg, from about 0.9 mg/kg to about 10 mg/kg, from about 1 mg/kg to about 5 mg/kg, from about 100 mg/kg to about 500 mg/kg, from about 1.0 mg/kg to about 250 mg/kg, or from about 10.0 mg/kg to about 150 mg/kg, in one or more dose administrations daily, for one or several days (depending, of course, of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some particular embodiments, the amount is less than 10,000 mg per day with a range, for example, of 750 mg to 9,000 mg per day.

The effective amount may be less than 1 mg/kg/day, less than 500 mg/kg/day, less than 250 mg/kg/day, less than 100 mg/kg/day, less than 50 mg/kg/day, less than 25 mg/kg/day, less than 10 mg/kg/day, or less than 5 mg/kg/day. It may alternatively be in the range of 1 mg/kg/day to 200 mg/kg/day. For example, regarding treatment of patients with COVID-19, the unit dosage may be an amount that reduces urine protein concentration by at least 40% as compared to an untreated subject or patient. In another embodiment, the unit dosage is an amount that reduces urine protein concentration to a level that is within ±10% of the urine protein level of a healthy subject or patient.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 1 mg/kg/body weight to about 5 mg/kg/body weight, a range of about 5 mg/kg/body weight to about 100 mg/kg/body weight, about 5 microgram/kg/body weight to about 500 milligram/kg/body weight, etc., can be administered, based on the numbers described above.

In certain embodiments, a pharmaceutical composition of the present invention may comprise, for example, at least about 0.1% of a compound of the present invention. In other embodiments, the compound of the present invention may comprise between about 2% to about 75% of the weight of the unit, or between about 25% to about 60%, for example, and any range derivable therein.

Single or multiple doses of the agents are contemplated. Desired time intervals for delivery of multiple doses can be determined by one of ordinary skill in the art employing no more than routine experimentation. As an example, subjects or patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the subject or patient has eaten or will eat.

Non-limiting specific formulations include CDDO-Me polymer dispersions (see U.S. Patent Application Publication No. 2009/0048204, filed Aug. 13, 2008, which is incorporated herein by reference). Some of the formulations reported therein exhibited higher bioavailability than either the micronized Form A or nanocrystalline Form A formulations. Additionally, the polymer dispersion-based formulations demonstrated further surprising improvements in oral bioavailability relative to the micronized Form B formulations. For example, the methacrylic acid copolymer, Type C and HPMC-P formulations showed the greatest bioavailability in the subject monkeys.

### IV. Combination Therapy

In addition to being used as a monotherapy, the compounds of the present invention may also find use in combination therapies. Effective combination therapy may be achieved with a single composition or pharmacological formulation that includes both agents, or with two distinct compositions or formulations, administered at the same time, wherein one composition includes a compound of this invention, and the other includes the second agent(s). Alternatively, the therapy may precede or follow the other agent treatment by intervals ranging from minutes to months.

Various combinations may be employed, such as when a compound of the present invention is “A” and “B” represents a secondary agent, non-limiting examples of which are described below:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

It is contemplated that other therapeutic agents may be used in conjunction with the treatments of the current invention. In some embodiments, the present invention contemplates the use of one or more other therapies for the treatment of COVID-19 including the use of an anti-viral agent, an anti-platelet drug, an anti-coagulation agent, or a steroid. In some embodiments, the present invention contemplates the use of one or more other therapies for the treatment of COVID-19 including the use of a SARS-CoV-2 protease inhibitor, anti-platelet drugs, an anti-coagulation agent, a human type I interferon, a corticosteroid, or remdesivir.

In some embodiment, the anti-viral drug is baloxavir, chloroquine phosphate, favipiravir, a viral protease inhibitor (e.g., lopinavir, atazanavir, darunavir, nelfinavir, tironavir, saquinavir, tipranavir), hydroxychloroquine, a neuraminidase inhibitor (e.g., oseltamivir), remdesivir, GS-441524, GS-443902, a SARS-CoV-2-specific monoclonal antibody (e.g., casirivimab (REGN10933), imdevimab (REGN10987), bamlanivimab (LY-CoV555), etesevimab (LY-CoV016), VIR-7831 (GSK4182136), AZD7442, COVID-GUARD (STI-1499), COVI-AMG (STI-2020)), or umifenovir.

In some embodiments, the anti-platelet drug is aspirin, an ADP receptor antagonist (e.g., ticlopidine, clopidogrel, cangrelor, prasugrel, ticagrelor, thienopyridine), or a glycoprotein IIb/IIIa receptor inhibitor (e.g., abciximab, eptifibatide, ticofiban).

In some embodiment, the anti-coagulation agent is rivaroxaban, apixaban, dipyridamole, cilostazol, atromentin, edoxaban, fondaprinux, betrixaban, letaxaban, eribaxaban, hirudin, a thrombin inhibitor (e.g., lepirudin, desirudin, dabigatran, bivalirudin, ximelagatran), argatroban, batroxobin, hementin, low molecular weight heparin, unfractionated heparin, vitamin E, or a vitamin K antagonist (e.g., warfarin (Coumadin), acenocoumarol, phenprocoumon, phenindione).

Human type I interferons (IFNs) are a large subgroup of interferon proteins that help regulate the activity of the immune system. The mammalian types are designated IFN-α (alpha), IFN-β (beta), IFN-κ (kappa), IFN-δ (delta), IFN-ε (epsilon), IFN-τ (tau), IFN-ω (omega), and IFN-ζ (zeta, also known as limitin). Type I interferons have shown efficacy against the replication of various viruses, included Zika virus, chikungunya virus, flaviviruses, and hepatitis C virus. “Interferon compounds” include interferon-alpha, interferon-alpha analogues, interferon-alpha derivatives, interferon-alpha conjugates, interferon beta, interferon-beta analogues, interferon-beta derivatives, interferon-beta conjugates and mixtures thereof. The whole protein or its fragments can be fused with other peptides and proteins such as immunoglobulins and other cytokines. Interferon-alpha and interferon-beta conjugates may represent, for example, a composition comprising interferon-beta coupled to a non-naturally occurring polymer comprising a polyalkylene glycol moiety. Preferred interferon compounds include Roferon®, Intron®, Alferon®, Infergen®, Omniferon®, Alfacon-1, interferon-alpha, interferon-alpha analogues, pegylated interferon-alpha, polymerized interferon-alpha, dimerized interferon-alpha, interferon-alpha conjugated to carriers, interferon-alpha as oral inhalant, interferon-alpha as injectable compositions, interferon-alpha as a topical composition, Roferon® analogues, Intron® analogues, Alferon® analogues, and Infergen® analogues, Omniferon® analogues, Alfacon-1 analogues, interferon beta, Avonex™, Betaseron™, Betaferon™, Rebif™, interferon-beta analogues, pegylated interferon-beta, polymerized interferon-beta, dimerized interferon-beta, interferon-beta conjugated to carriers, interferon-beta as oral inhalant, interferon-beta as an injectable composition, interferon-beta as a topical composition, Avonex™ analogues, Betaseron™ Betaferon™ analogues, and Rebif™ analogues. Alternatively, agents that induce interferon-alpha or interferon-beta production or mimic the action of interferon-alpha or interferon-beta may also be employed. Interferon inducers include tilorone, poly(I)-poly(C), imiquimod, cridanimod, bropirimine.

It is contemplated that other agents may be used in combination with certain aspects of the present invention to improve the therapeutic efficacy of treatment. These additional agents include an anti-viral, a corticosteroid (e.g., dexamethasone, hydrocortisone, methylprednisolone, prednisone, budesonide), an anti-rheumatic drug (e.g., anakinra, baricitinib, sarilumab, tocilizumab), chloroquine, decitabine, hydroxychloroquine, remdesivir, favipiravir, lopinavir, ritonavir, ascorbic acid, a macrolide antibiotic (e.g., azithromycin), colchicine, a prostacyclin (e.g., epoprostenol. iloprost), an interferon (e.g., IFN beta-1a, IFN beta-1b, peginterferon beta-1a, IFN alpha, early IFN alpha-2b), nitric oxide, an antineoplastic agent (e.g., siltuximab, ruxolitinib), sirolimus, vitamin D, zinc, an ACE inhibitor, an angiotensin II receptor blocker, an anticoagulant, famotidine, fluvoxamine, HMG-CoA reductase inhibitors (e.g., statins), immune globulin (e.g., pooled plasma from adult human blood, pooled plasma from individuals who have recovered from COVID-19), an anti-helminthic (e.g., ivermectin, niclosamide), nitazoxanide, a nonsteroidal anti-inflammatory agent (e.g., ibuprofen, indomethacin), and a thrombolytic agent (e.g., t-PA).

### V. Characteristics of Patients Who Might be Excluded from Treatment with Bardoxolone Methyl

Several clinical studies have shown that treatment with bardoxolone methyl improved markers of renal function (including estimated glomerular filtration rate, or eGFR), insulin resistance, and endothelial dysfunction (Pergola et al., 2011). These observations led to the initiation of a large Phase 3 trial (BEACON) of bardoxolone methyl in patients with stage 4 CKD and type 2 diabetes. The primary endpoint in the BEACON trial was a composite of progression to end-stage renal disease (ESRD) and all-cause mortality. This trial was terminated due to excess severe adverse events and mortality in the group of patients treated with bardoxolone methyl.

As discussed below, subsequent analysis of the data from the BEACON trial showed that most of the severe adverse events and mortality involved heart failure and were highly correlated with the presence of one or more risk factors including: (a) elevated baseline levels of B-type natriuretic peptide (BNP; e.g., >200 pg/mL); (b) baseline eGFR <20; (c) history of left-sided heart disease; (d) high baseline albumin-to-creatinine ratio (ACR; e.g., >300 mg/g as defined by dipstick proteinuria of 3+); and (e) advanced age (e.g., >75 years). The analysis indicated that heart failure events were likely related to the development of acute fluid overload in the first three to four weeks of bardoxolone methyl treatment and that this was potentially due to inhibition of endothelin-1 signaling in the kidney. A previous trial of an endothelin receptor antagonist in stage 4 CKD patients was terminated due to a pattern of adverse events and mortality very similar to that found in the BEACON trial. Subsequent non-clinical studies confirmed that bardoxolone methyl, at physiologically relevant concentrations, inhibits endothelin-1 expression in renal proximal tubule epithelial cells and inhibits endothelin receptor expression in human mesangial and endothelial cells. Accordingly, patients at risk of adverse events from inhibition of endothelin signaling may be excluded from future clinical use of bardoxolone methyl.

The present invention concerns new methods of treating COVID-19 symptoms and complications that include modification of the glomerular basement membrane as a significant contributing factor. It also concerns the preparation of pharmaceutical compositions for the treatment of such disorders. In some embodiments of the present invention, patients for treatment are selected on the basis of several criteria: (1) diagnosis of a disorder that involves endothelial dysfunction as a significant contributing factor; (2) lack of elevated levels of B-type natriuretic peptide (BNP; e.g., BNP titers must be <200 pg/mL); (3) lack of chronic kidney disease (e.g., eGFR >60) or lack of advanced chronic kidney disease (e.g., eGFR >45); (4) lack of a history of left-sided myocardial disease; and (5) lack of a high ACR (e.g., ACR below 300 mg/g). In some embodiments of the invention, patients with a diagnosis of type 2 diabetes are excluded. In some embodiments of the invention, patients with a diagnosis of cancer are excluded. In some embodiments, patients of advanced age (e.g., >75 years) are excluded. In some embodiments, patients are closely monitored for rapid weight gain suggestive of fluid overload. For example, patients may be instructed to weigh themselves daily for the first four weeks of treatment and contact the prescribing physician if increases of greater than five pounds are observed.

Provided herein are compounds that may be used to treat a coronavirus (e.g., a beta-coronavirus; e.g., SARS-CoV-2) infection, as well as treat or prevent symptoms or complications thereof, in a patient having a baseline BNP level that is not elevated (e.g., is less than or equal to 200 pg/mL). Provided herein are compounds that may be used to treat a coronavirus (e.g., a beta-coronavirus; e.g., SARS-CoV-2) infection, as well as treat or prevent symptoms or complications thereof, in a patient having a baseline eGFR of greater than 20, greater than 25, greater than 30, greater than 35, greater than 40, greater than 45, greater than 50, greater than 55, or greater than 60 mL/min/1.73 m². Provided herein are compounds that may be used to treat a coronavirus (e.g., a beta-coronavirus; e.g., SARS-CoV-2) infection, as well as treat or prevent symptoms or complications thereof, in a patient having a baseline albumin-to-creatinine ratio that is not high (e.g., is less than or equal to 300 mg/g). Provided herein are compounds that may be used to treat a coronavirus (e.g., a beta-coronavirus; e.g., SARS-CoV-2) infection, as well as treat or prevent symptoms or complications thereof, in a patient without a history of left-sided myocardial disease.

A. BEACON Study

1. Design of Study

Study 402-C-0903, titled “Bardoxolone Methyl Evaluation in Patients with Chronic Kidney Disease and Type 2 Diabetes: The Occurrence of Renal Events” (BEACON) was a phase 3, randomized, double-blind, placebo-controlled, parallel-group, multinational, multicenter study designed to compare the efficacy and safety of bardoxolone methyl (BARD) to placebo (PBO) in patients with stage 4 chronic kidney disease and type 2 diabetes. A total of 2,185 patients were randomized 1:1 to once-daily administration of bardoxolone methyl (20 mg) or placebo. The primary efficacy endpoint of the study was the time-to-first event in the composite endpoint defined as end-stage renal disease (ESRD; need for chronic dialysis, renal transplantation, or renal death) or cardiovascular (CV) death. The study had three secondary efficacy endpoints: (1) change in estimated glomerular filtration rate (eGFR); (2) time-to-first hospitalization for heart failure or death due to heart failure; and (3) time-to-first event of the composite endpoint consisting of non-fatal myocardial infarction, non-fatal stroke, hospitalization for heart failure, or cardiovascular death.

A subset of the BEACON patients consented to additional 24-hour assessments including ambulatory blood pressure monitoring (ABPM) and 24-hour urine collections. An independent Events Adjudication Committee (EAC), blinded to study treatment assignment, evaluated whether renal events, cardiovascular events, and neurological events met the pre-specified definitions of the primary and secondary endpoints. An IDMC, consisting of external clinical experts supported by an independent statistical group, reviewed unblinded safety data throughout the study and made recommendations as appropriate.

2. Demographics and Baseline Characteristics of the Population

Table 4 presents summary statistics on select demographic and baseline characteristics of patients enrolled in BEACON. Demographic characteristics were comparable across the two treatment groups. In all treatment groups combined, the average age was 68.5 years and 57% of the patients were male. The bardoxolone methyl arm had slightly more patients in the age subgroup ≥75 years than the placebo arm (27% in bardoxolone methyl arm versus 24% in the placebo arm). Mean weight and BMI across both treatment groups was 95.2 kg and 33.8 kg/m², respectively. Baseline kidney function was generally similar in the two treatment groups; mean baseline eGFR, as measured by the 4-variable Modified Diet in Renal Disease (MDRD) equation, was 22.5 mL/min/1.73 m² and the geometric mean albumin/creatinine ratio (ACR) was 215.5 mg/g for the combined treatment groups.

TABLE 4
Select Demographics and Baseline Characteristics of Bardoxolone Methyl
(BARD) versus Placebo (PBO) Patients in BEACON (ITT Population)

| | BARD | | PBO | | Total | |
|---|---|---|---|---|---|---|
| | N = 1088 | | N = 1097 | | N = 2185 | |
| Sex, n (%) | | | | | | |
| Male | 626 | (58) | 625 | (57) | 1251 | (57) |
| Female | 462 | (42) | 472 | (43) | 934 | (43) |
| Age at informed consent (years) | | | | | | |
| n | 1088 | | 1097 | | 2185 | |
| Mean (SD) | 68.9 | (9.7) | 68.2 | (9.4) | 68.5 | (9.6) |
| Range (min, max) | 32, 92 | | 29, 93 | | 29, 93 | |
| Age subgroup, n (%) | | | | | | |
| <75 | 786 | (72) | 829 | (76) | 1615 | (74) |
| ≥75 | 302 | (27) | 268 | (24) | 570 | (26) |
| Weight (kg) | | | | | | |
| n | 1087 | | 1097 | | 2184 | |
| Mean (SD) | 95.1 | (22.0) | 95.3 | (21.1) | 95.2 | (21.5) |
| Range (min, max) | 46, 194 | | 45, 186 | | 45, 194 | |
| BMI (kg/m²) | | | | | | |
| n | 1087 | | 1097 | | 2184 | |
| Mean (SD) | 33.7 | (7.1) | 33.9 | (7.2) | 33.8 | (7.1) |
| Range (min, max) | 19, 93 | | 19, 64 | | 19, 93 | |
| eGFR (mL/min/1.73 m²) mean (SD) | | | | | | |
| n | 1088 | | 1097 | | 2185 | |
| Mean (SD) | 22.4 | (4.3) | 22.5 | (4.6) | 22.5 | (4.5) |
| Range (min, max) | 13, 34 | | 13, 58 | | 13, 58 | |
| eGFR MDRD subgroup, n (%) | | | | | | |
| 15-<20 | 325 | (30) | 347 | (32) | 672 | (31) |
| 20-<25 | 399 | (37) | 366 | (33) | 765 | (35) |
| 25-<30 | 311 | (29) | 318 | (29) | 629 | (29) |
| ACR (mg/g) geometric mean | | | | | | |
| n | 1088 | | 1097 | | 2185 | |
| Geometric mean | 210.4 | | 220.7 | | 215.5 | |
| (95% CI) | (188, 236) | | (196, 249) | | (198, 234) | |
| Range (min, max) | <1, 4581 | | <1, 79466 | | <1, 79466 | |
| ACR subgroup, n (%) | | | | | | |
| <30 | 200 | (18) | 211 | (19) | 411 | (19) |
| 30-300 | 348 | (32) | 308 | (28) | 656 | (30) |
| >300 | 540 | (50) | 578 | (53) | 1118 | (51) |

Patients were administered placebo or 20 mg of bardoxolone methyl once daily.

B. BEACON Results

1. Effect of Bardoxolone Methyl on eGFR

On average, bardoxolone methyl patients had expected increases in eGFR that occurred by Week 4 of treatment and remained above baseline through Week 48. In contrast, placebo-treated patients on average had unchanged or slight decreases from baseline. The proportion of patients with eGFR declines was markedly reduced in bardoxolone methyl-versus placebo-treated patients. The eGFR trajectories and the proportions of decliners observed in BEACON after one year of treatment were consistent with modeled expectations and results from the BEAM study (RTA402-C-0804). As shown in Table 5, the number of patients who experienced a renal and urinary disorder serious adverse event (SAE) was lower in the bardoxolone methyl group than in the placebo group (52 vs. 71, respectively). Additionally, and as discussed in the following section, slightly fewer ESRD events were observed in the bardoxolone methyl group than in the placebo group. Collectively, these data suggest that bardoxolone methyl treatment did not worsen renal status acutely or over time.

TABLE 5
Incidence of Treatment-Emergent Serious Adverse Events in BEACON
within Each Primary System Organ Class (Safety Population)

| | | | Bardoxolone | |
|---|---|---|---|---|
| | Placebo | | methyl | |
| | N = 1093 | | N = 1092 | |
| MedDRA System Organ Class | n (%) | | n (%) | |
| Patients with any serious adverse event | 295 | (27) | 363 | (33) |
| Number of serious adverse events | 557 | | 717 | |
| Cardiac disorders | 84 | (8) | 124 | (11) |
| Infections and infestations | 63 | (6) | 79 | (7) |
| Renal and urinary disorders | 71 | (6) | 52 | (5) |
| Metabolism and nutrition disorders | 42 | (4) | 51 | (5) |
| Gastrointestinal disorders | 39 | (4) | 46 | (4) |
| Respiratory, thoracic and mediastinal | 32 | (3) | 43 | (4) |
| disorders | | | | |
| Nervous system disorders | 35 | (3) | 37 | (3) |
| General disorders and administration site | 20 | (2) | 29 | (3) |
| conditions | | | | |
| Vascular disorders | 18 | (2) | 20 | (2) |
| Injury, poisoning and procedural | 17 | (2) | 19 | (2) |
| complications | | | | |
| Musculoskeletal and connective tissue | 13 | (1) | 21 | (2) |
| disorders | | | | |
| Blood and lymphatic system disorders | 11 | (1) | 20 | (2) |
| Neoplasms benign, malignant and | 10 | (1) | 11 | (1) |
| unspecified (incl. cysts and polyps) | | | | |
| Hepatobiliary disorders | 8 | (1) | 4 | (<1) |
| Psychiatric disorders | 3 | (<1) | 3 | (<1) |
| Eye disorders | 2 | (<1) | 3 | (<1) |
| Investigations | 2 | (<1) | 3 | (<1) |
| Reproductive system and breast disorders | 3 | (1) | 2 | (<1) |
| Skin and subcutaneous tissue disorders | 1 | (<1) | 4 | (<1) |
| Ear and labyrinth disorders | 1 | (<1) | 3 | (<1) |
| Endocrine disorders | 1 | (<1) | 1 | (<1) |

| | | | |
|---|---|---|---|
| Immune system disorders | 0 | 2 | (<1) |
| Surgical and medical procedures | 0 | 2 | (<1) |

Table includes only serious adverse events with onset more than 30 days after a patient's last dose of study drug. Column header counts and denominators are the number of patients in the safety population. Each patient is counted at most once in each System Organ Class and Preferred Term.

2. Primary Composite Outcome in BEACON

Table 6 provides a summary of adjudicated primary endpoints that occurred on or before the date of study termination (Oct. 18, 2012). Despite the slight reduction in the number of ESRD events in the bardoxolone methyl vs. placebo treatment groups, the number of composite primary endpoints was equal in the two treatment groups (HR=0.98) due to a slight increase in cardiovascular death events, as depicted in plots of time-to-first composite primary event analysis.

TABLE 6
Adjudicated Primary Endpoints in Bardoxolone Methyl (BARD)
vs. Placebo (PBO) Patients in BEACON (ITT Population)

| | PBO | BARD | Hazard | |
|---|---|---|---|---|
| | N = 1097 | N = 1088 | ratio | p- |
| | n (%) | n (%) | (95% CI)^{a} | value^{b} |
| Composite primary | 69 (6) | 69 (6) | 0.98 (0.70, | 0.92 |
| efficacy outcome | | | 1.37) | |
| Patient's first event | | | | |
| End stage renal disease | 51 (5) | 43 (4) | | |
| (ESRD) | | | | |
| Chronic dialysis | 47 (4) | 40 (4) | | |
| Renal transplant | 3 (<1) | 1 (<1) | | |
| Renal death | 1 (<1) | 2 (<1) | | |
| CV death | 18 (2) | 26 (2) | | |

^{a}Hazard ratio (bardoxolone methyl/placebo) and 95% confidence interval (CI) were estimated using a Cox proportional hazards model with treatment group, continuous baseline eGFR, and continuous baseline log ACR as covariates. Breslow's method of handling ties in event time was used.
^{b}Treatment group comparisons used SAS's Type 3 chi-square test and two-sided p-value associated with the treatment group variable in the Cox proportional hazards model.

C. Effects of Bardoxolone Methyl on Heart Failure and Blood Pressure

1. Adjudicated Heart Failure in BEACON

The data in Table 7 present a post-hoc analysis of demographic and select laboratory parameters of BEACON patients stratified by treatment group and occurrence of an adjudicated heart failure event. The number of patients with heart failure includes all events through last date of contact (ITT Population).

Comparison of baseline characteristics of patients with adjudicated heart failure events revealed that both bardoxolone methyl-treated and placebo-treated patients with heart failure were more likely to have had a prior history of cardiovascular disease and heart failure and had higher baseline values for B-type natriuretic peptide (BNP) and QTc interval with Fredericia correction (QTcF). Even though the risk for heart failure was higher in the bardoxolone methyl-treated patients, these data suggest that development of heart failure in both groups appeared to be associated with traditional risk factors for heart failure. Baseline ACR was significantly higher in bardoxolone methyl-treated patients with heart failure events than those without. Also of note, the mean baseline level of BNP in patients who experienced heart failure in both treatment groups was meaningfully elevated and suggested that these patients were likely retaining fluid and in sub-clinical heart failure prior to randomization.

TABLE 7
Select Demographic and Baseline Characteristics for Bardoxolone Methyl vs.
Placebo Patients Stratified by Heart Failure Status

| | | | Without Heart | | | |
|---|---|---|---|---|---|---|
| | With Heart Failure | | Failure | | Total | |
| | BARD | PBO | BARD | PBO | BARD | PBO |
| Patients | (N = 103) | (N = 57) | (N = 985) | (N = 1040) | (N = 1088) | (N = 1097) |
| Age (years), Mean ± | 70.3 ± 9 | 69.2 ± 8.2 | 68.7 ± 9.8 | 68.1 ± 9.5 | 68.9 ± 9.7 | 68.2 ± 9.4 |
| SD | | | | | | |
| History of CVD, N | 80 (78)ª | 47 (82)^{b} | 529 (54) | 572 (55) | 609 (56) | 619 (56) |
| (%) | | | | | | |
| History of HF, N (%) | 36 (35)^{a} | 21 (37)^{b} | 130 (13) | 133 (13) | 166 (15) | 154 (14) |
| History of MI, N (%) | 33 (32)ª | 22 (39)^{b} | 185 (19) | 188 (18) | 218 (20) | 210 (19) |
| History of A-FIB, N | 4 (4)ª | 3 (5) | 46 (5) | 40 (4) | 50 (5) | 43 (4) |
| (%) | | | | | | |
| Concomitant Med | | | | | | |
| Use, N (%) | | | | | | |
| ACEi/ARB | 35 (34)ª | 16 (28)^{b} | 659 (67) | 701 (67) | 694 (64) | 717 (65) |
| Diuretic | 39 (38)ª | 15 (26)^{b} | 528 (54) | 586 (56) | 567 (52) | 601 (55) |
| Beta-Blocker | 38 (37)ª | 23 (40) | 482 (49) | 506 (49) | 520 (48) | 529 (48) |
| Statin | 57 (55) | 26 (46)^{b} | 640 (65) | 721 (69) | 697 (64) | 747 (68) |
| Calcium Channel | 25 (24)ª | 17 (30)^{b} | 406 (41) | 467 (45) | 431 (40) | 484 (44) |
| Blocker | | | | | | |
| eGFR (mL/min/1.73 | 21.7 ± 4.6 | 22.2 ± 4.7 | 22.5 ± 4.2 | 22.5 ± 4.6 | 22.4 ± 4.3 | 22.5 ± 4.6 |
| m²), | | | | | | |
| Mean ± SD | | | | | | |
| ACR (mg/g), Geo | 353.9ª | 302.0 | 199.3 | 216.9 | 210.4 | 220.7 |
| Mean | | | | | | |
| SBP (mmHg), Mean ± | 139.5 ± 13.3 | 142.3 ± 11.2 | 139.5 ± 11.6 | 139.6 ± 11.8 | 139.5 ± 11.7 | 139.8 ± 11.8 |
| SD | | | | | | |
| DBP (mmHg), Mean ± | 66.4 ± 9.1ª | 69.1 ± 8.8 | 70.4 ± 8.7 | 70.8 ± 8.6 | 70.1 ± 8.8 | 70.7 ± 8.7 |
| SD | | | | | | |
| BNP (pg/mL) | | | | | | |
| Mean ± SD | 526.0 ± 549.4ª | 429.8 ± 434.3^{b} | 223.1 ± 257.5 | 232.3 ± 347.1 | 251.2 ± 309.1 | 242.7 ± 354.7 |
| >100, N (%) | 78 (76)ª | 43 (75)^{b} | 547 (56) | 544 (52) | 625 (57) | 587 (54) |
| QTcF (ms) | | | | | | |
| Mean ± SD | 447.9 ± 31.2^{a,c} | 432.5 ± 27.6^{b} | 425.3 ± 27.8 | 424.7 ± 27.9 | 427.4 ± 28.9 | 425.1 ± 28 |
| >450, N (%) | 40 (39)ª | 14 (25) | 170 (17) | 167 (16) | 210 (19) | 181 (16) |

^{a}p < 0.05 for BARD patients with HF vs. BARD patients without HF
^{b}p < 0.05 for PBO patients with HF vs. PBO patients without HF
^{c}p < 0.05 for BARD vs. PBO patients with HF

2. Assessment of Clinical Parameters Associated with BNP Increases

As a surrogate of fluid retention, a post-hoc analysis was performed on a subset of patients for whom BNP data were available at baseline and Week 24. Patients in the bardoxolone methyl arm experienced a significantly greater increase in BNP than patients in the placebo arm (Mean±SD: 225±598 vs. 34±209 pg/mL, p<0.01). Also noted was a higher proportion of bardoxolone methyl- vs. placebo-treated patients with increases in BNP at Week 24 (Table 8).

BNP increases at Week 24 did not appear to be related to baseline BNP, baseline eGFR, changes in eGFR, or changes in ACR. However, in bardoxolone methyl-treated patients only, baseline ACR was significantly correlated with Week 24 changes from baseline in BNP, suggesting that the propensity for fluid retention may be associated with baseline severity of renal dysfunction, as defined by albuminuria status, and not with the general changes in renal function, as assessed by eGFR (Table 9).

Further, these data suggest that increases in eGFR, which are glomerular in origin, are distinct anatomically, as sodium and water regulation occurs in the renal tubules.

TABLE 8
Analysis of BNP and eGFR Values of Bardoxolone
Methyl vs. Placebo Patients Stratified by
Changes from Baseline in BNP at Week 24

| WK 24 BNP | Treat- | | Median | Mean | Mean WK 24 |
|---|---|---|---|---|---|
| Change | ment | N | BL BNP | BL eGFR | ΔeGFR |
| <25% | PBO | 131 | 119.0 | 23.5 | −0.6 |
| Increase | BARD | 84 | 187.0 | 22.3 | 6.1 |
| 25% to 100% | PBO | 48 | 102.5 | 22.0 | 0.4 |
| Increase | BARD | 45 | 119.0 | 22.7 | 5.5 |
| ≥100% | PBO | 37 | 143.5 | 23.1 | 0.1 |
| Increase | BARD | 82 | 155.0 | 21.9 | 7.6 |

Post-hoc analysis of changes in BNP in BEACON at Week 24.

TABLE 9
Correlations between Changes from Baseline
in BNP at Week 24 and Baseline ACR in Bardoxolone
Methyl vs. Placebo Patients in BEACON

| Treatment | N | Correlation Coefficient | P-value |
|---|---|---|---|
| PBO | 216 | 0.05 | 0.5 |
| BARD | 211 | 0.20 | <0.01 |

Post-hoc analysis of changes in BNP in BEACON at Week 24. Only patients with baseline and Week 24 BNP values included in analysis.

3. Serum Electrolytes

No clinically meaningful changes were noted in serum potassium or serum sodium for the subset of patients with 24-hr urine collections (Table 10). The change in serum magnesium levels in bardoxolone methyl-treated patients was consistent with changes observed in prior studies.

TABLE 10
Week 4 Changes from Baseline in Serum Electrolytes in Bardoxolone Methyl vs.
Placebo 24-hour ABPM Sub-Study Patients

| | | Serum Potassium | | | Serum Sodium | | | Serum Magnesium | | |
|---|---|---|---|---|---|---|---|---|---|---|
| | | (mmol/L) | | | (mmol/L) | | | (m Eq/L) | | |
| | | BL | WK4 | WK4 Δ | BL | WK4 | WK4 Δ | BL | WK4 | WK4 Δ |
| PBO | n | 88 | 87 | 87 | 88 | 87 | 87 | 88 | 87 | 87 |
| | Mean ± SE | 4.8 ± 0.1 | 4.7 ± 0.1 | −0.10 ± 0.04* | 140.2 ± 0.2 | 139.7 ± 0.3 | −0.3 ± 0.2 | 1.72 ± 0.03 | 1.69 ± 0.03 | −0.03 ± 0.02 |
| BARD | n | 83 | 77 | 77 | 83 | 77 | 77 | 83 | 77 | 77 |
| | Mean ± SE | 4.7 ± 0.1 | 4.8 ± 0.1 | 0.10 ± 0.05*^{†} | 140.1 ± 0.3 | 140.3 ± 0.3 | 0.2 ± 0.3 | 1.74 ± 0.02 | 1.53 ± 0.03 | −0.21 ± 0.02*^{†} |

Data include only BEACON patients enrolled in the 24-hour ABPM sub-study. Changes in serum electrolyte values only calculated for patients with baseline and Week 4 data.
*p < 0.05 for Week 4 versus baseline values within each treatment group;
^{†}p < 0.05 for Week 4 changes in BARD vs. PBO patients.

4. 24-hour Urine Collections

A subset of patients consented to additional 24-hr assessments (sub-study) of ambulatory blood pressure monitoring (ABPM) and 24-hr urine collection at selected visits. Urinary sodium excretion data from BEACON sub-study patients revealed a clinically meaningful reduction in urine volume and excretion of sodium at Week 4 relative to baseline in the bardoxolone methyl-treated patients (Table 11). These decreases were significantly different from Week 4 changes in urine volume and urinary sodium observed in placebo-treated patients. Also of note, reductions in serum magnesium were not associated with renal loss of magnesium.

Additionally, in a pharmacokinetic study in patients with type 2 diabetes and stage 3b/4 CKD administered bardoxolone methyl for eight weeks (402-C-1102), patients with stage 4 CKD had significantly greater reductions of urinary sodium and water excretion than stage 3b CKD patients (Table 12).

TABLE 11
Week 4 Changes from Baseline in 24-hour Urine Volume, Urinary Sodium, and Urinary Potassium in Bardoxolone Methyl vs.
Placebo 24-hour ABPM Sub-Study Patients

| | | Urine Volume | | | Urinary Sodium | | | Urinary Potassium | | | Urinary Magnesium | | |
|---|---|---|---|---|---|---|---|---|---|---|---|---|---|
| | | (mL) | | | (mmol/24 h) | | | (mmol/24 h) | | | (mmol/24 h) | | |
| | | BL | WK4 | WK4 Δ | BL | WK4 | WK4 Δ | BL | WK4 | WK4 Δ | BL | WK4 | WK4 Δ |
| PBO | n | 87 | 72 | 71 | 81 | 68 | 62 | 81 | 68 | 62 | 59 | 53 | 46 |
| | Mean ± SE | 2053 ± 82 | 1928 ± 89 | −110 ± 71 | 160 ± 8 | 145 ± 8 | −11 ± 9 | 55 ± 3 | 52 ± 3 | −3 ± 3 | 7.5 ± 0.5 | 6.0 ± 0.5 | −0.6 ± 0.4 |
| BARD | n | 82 | 64 | 63 | 77 | 61 | 57 | 77 | 61 | 57 | 56 | 43 | 40 |
| | Mean ± SE | 2024 ± 83 | 1792 ± 84 | −247 ± 71* | 164 ± 9 | 140 ± 9 | −27 ± 9* | 60 ± 3 | 52 ± 2 | −7 ± 3* | 7.0 ± 0.4 | 6.0 ± 0.4 | −0.9 ± 0.5 |

Data include only BEACON patients enrolled in the 24-hour ABPM sub-study. Changes at Week 4 only calculated for patients with baseline and Week 4 data.
*p < 0.05 for Week 4 versus baseline values within each treatment group;
† p < 0.05 for Week 4 changes in BARD versus PBO patients.

TABLE 12
Week 8 Changes from Baseline in 24-h Urine Volume and 24-h
Urinary Sodium Bardoxolone Methyl-treated Patients Grouped
by CKD Severity (from a Patient Pharmacokinetic Study)

| | | Urine Volume (mL) | | Urinary Sodium (mmol/24 h) | |
|---|---|---|---|---|---|
| CKD Stage | N | WK 8 Δ | p-value | WK 8 Δ | p-value |
| Stage 3b | 9 | 355 | 0.04 | −12 | 0.02 |
| Stage 4 | 6 | −610 | | −89 | |

Patients were treated with 20 mg bardoxolone methyl once daily for 56 consecutive days; post-treatment follow-up visit occurred on Study Day 84. Data are means. Data include patients with baseline and Week 8 data.

5. Hospital Records from EAC Adjudication Packets

The first scheduled post-baseline assessment in BEACON was at Week 4. Since many of the heart failure events occurred prior to Week 4, the clinical database provides limited information to characterize these patients. Post-hoc review of the EAC case packets for heart failure cases that occurred prior to Week 4 was performed to assess clinical, vitals, laboratory, and imaging data collected at the time of the first heart failure event (Tables 13 and 14).

Examination of these records revealed common reports of rapid weight gain immediately after randomization, dyspnea and orthopnea, peripheral edema, central/pulmonary edema on imaging, elevated blood pressure and heart rate, and preserved ejection fraction. The data suggest that heart failure was caused by rapid fluid retention concurrent with preserved ejection fraction and elevated blood pressure. The preserved ejection fraction is consistent with clinical characteristics of heart failure caused by diastolic dysfunction stemming from ventricular stiffening and impaired diastolic relaxation. This collection of signs and symptoms differs in clinical characteristics from heart failure with reduced ejection fraction, which occurs because of weakened cardiac pump function or contractile impairment (Vasan et al., 1999). Therefore, rapid fluid accumulation in patients with stuff ventricles and minimal renal reserve likely resulted in increased fluid back-up into the lungs and the noted clinical presentation.

Baseline central laboratory values from the clinical database were compared to local laboratory values obtained on admission for heart failure that were included in the EAC packets. Unchanged serum creatinine, sodium, and potassium concentrations in bardoxolone methyl-treated patients with heart failure events that occurred within the first four weeks after randomization (Table 14) suggest that heart failure was not associated with acute renal function decline or acute kidney injury. Overall, the clinical data suggest that the etiology of heart failure is not caused by a direct renal or cardiotoxic effect, but is more likely to be due to sodium and fluid retention.

TABLE 13
Post-Hoc Analysis of Cardiovascular Parameters of Bardoxolone Methyl vs.
Placebo Patients with Heart Failure Events Occurring Within First Four Weeks of Treatment

| | | LVEF | SBP (mmHg) | | | DBP (mmHg) | | | Heart Rate (bpm) | | |
|---|---|---|---|---|---|---|---|---|---|---|---|
| | | HF | BL | HF | Δ | BL | HF | Δ | BL | HF | Δ |
| PBO | n | 4 | 8 | 6 | 6 | 8 | 6 | 6 | 8 | 5 | 5 |
| | Mean ± SE | 49% ± 6% | 141 ± 5 | 148 ± 11 | −4.7 ± 7.2 | 65 ± 3 | 65 ± 5 | 1.2 ± 3.6 | 70 ± 3 | 65 ± 3 | −3.6 ± 2.9 |
| BARD | n | 23 | 42 | 33 | 33 | 42 | 34 | 34 | 42 | 32 | 32 |
| | Mean ± SE | 52% ± 2% | 142 ± 2 | 154 ± 4 | 10.5 ± 3.1 | 67 ± 2 | 75 ± 2 | 7.9 ± 2.1 | 67 ± 1 | 81 ± 3 | 14.5 ± 2.7 |

Post-hoc analyses of heart failure cases in BEACON. Vital signs at baseline calculated from the average of three standard cuff measurements. Vital signs from HF hospitalization gathered from admission notes included in EAC Adjudication packets and represent singular assessments using different BP monitoring equipment. LVEF only assessed during HF hospitalization. Timing of HF admission calculated from event start date and treatment start date and varied from Weeks 0-4 for each patient.

TABLE 14
Post-Hoc Analysis of Serum Electrolytes of Bardoxolone Methyl vs. Placebo
Patients with Heart Failure Events Occurring Within First Four Weeks of Treatment

| | | Serum Creatinine | | | Serum Sodium | | | Serum Potassium | | |
|---|---|---|---|---|---|---|---|---|---|---|
| | | (mg/dL) | | | (mmol/L) | | | (mmol/L) | | |
| | | BL | HF | Δ | BL | HP | Δ | BL | HF | Δ |
| PBO | n | 8 | 8 | 8 | 8 | 8 | 8 | 8 | 8 | 8 |
| | Mean ± SE | 3.4 ± 0.2 | 3.3 ± 0.2 | −0.1 ± 0.2 | 140.0 ± 1.0 | 137.0 ± 1.0 | −2.5 ± 0.6 | 4.5 ± 0.2 | 4.4 ± 0.1 | −0.1 ± 0.2 |
| BARD | n | 42 | 38 | 38 | 42 | 30 | 30 | 42 | 34 | 34 |
| | Mean ± SE | 2.8 ± 0.1 | 2.7 ± 0.1 | −0.1 ± 0.1 | 140.0 ± 0.0 | 139.0 ± 1.0 | −1.0 ± 0.5 | 4.7 ± 0.1 | 4.8 ± 0.1 | 0.1 ± 0.1 |

Post-hoc analyses of heart failure cases in BEACON. Baseline clinical chemistries assessed at central laboratory. Clinical chemistries from HF hospitalization gathered from hospital notes included in EAC Adjudication packets and represent assessments made at different local laboratories.

6. Blood Pressure in BEACON

Mean changes from baseline in systolic and diastolic blood pressures for bardoxolone methyl-treated and placebo-treated patients, based on the average of triplicate standardized blood pressure cuff measurements collected at each visit. Blood pressure was increased in the bardoxolone methyl group relative to the placebo group, with mean increases of 1.9 mmHg in systolic and 1.4 mmHg in diastolic blood pressures noted in the bardoxolone methyl group by Week 4 (the first post-randomization assessment). The increases in systolic blood pressure (SBP) appeared to diminish by Week 32, while diastolic blood pressure (DBP) increases were sustained.

The Week 4 SBP and DBP increases in bardoxolone methyl-treated patients relative to placebo-treated patients were more apparent in the ABPM measurements. This difference in magnitude could be due to the different techniques that were used or to differences in baseline characteristics in the ABPM sub-study patients. Patients in the ABPM sub-study had a higher baseline ACR than the entire population. Regardless, the data demonstrate that bardoxolone methyl increased blood pressure in the BEACON patient population.

7. Blood Pressure Changes in Prior CKD Studies

In an open label, dose-ranging study in type 2 diabetic patients with stage 3b-4 CKD (402-C-0902), no dose-related trend in blood pressure changes or change at any individual dose level was noted following 85 consecutive days of treatment at doses ranging from 2.5 to 30 mg of bardoxolone methyl (amorphous dispersion formulation, as used in BEACON). Post-hoc analysis of blood pressure data stratified by CKD stage suggests that bardoxolone methyl-treated patients with stage 4 CKD tended to have increases in blood pressure relative to baseline levels, with the effect most appreciable in the three highest dose groups, whereas bardoxolone methyl-treated patients with stage 3b CKD had no apparent change (Table 15). Although sample sizes in the dose groups stratified by CKD stage are small, these data suggest that the effect of bardoxolone methyl treatment on blood pressure may be related to CKD stage.

Blood pressure values from a phase 2b study with bardoxolone methyl (BEAM, 402-C-0804), which used an earlier crystalline formulation of the drug and employed a titration design, were highly variable and despite noted increases in some bardoxolone methyl treatment groups, no clear dose-related trend was observed in blood pressure.

TABLE 15
Changes from Baseline in Systolic and Diastolic Blood Pressure
in Patients with Type 2 Diabetes and Stage 3b-4 CKD Stratified
by Baseline CKD Stage Dosed with Bardoxolone Methyl

| Dose (mg) | CKD Stage | N | ΔSBP | ΔDBP |
|---|---|---|---|---|
| 2.5 | 3b/4 | 14 | 0.1 ± 4.2 | 0.2 ± 1.8 |
| | 3b | 10 | 0 ± 4.4 | 1 ± 2 |
| | 4 | 4 | 0.3 ± 11 | −1.5 ± 3.9 |
| 5 | 3b/4 | 24 | −1.5 ± 2.3 | −1.4 ± 1.5 |
| | 3b | 19 | −2.1 ± 2 | −1.3 ± 1.4 |
| | 4 | 5 | 0.5 ± 9.1 | −1.4 ± 5.6 |
| 10 | 3b/4 | 24 | −2.4 ± 3.1 | 0.3 ± 1.3 |
| | 3b | 20 | −4.2 ± 3.4 | −0.3 ± 1.3 |
| | 4 | 4 | 6.1 ± 6.7 | 3.6 ± 4.5 |
| 15 | 3b/4 | 48 | 1.1 ± 2.3 | −1 ± 1.2 |
| | 3b | 26 | −2.2 ± 3.3 | −1.3 ± 1.5 |
| | 4 | 22 | 5 ± 2.8 | −0.6 ± 1.9 |
| 30 | 3b/4 | 12 | 7.2 ± 6.2 | 3.2 ± 2.2 |
| | 3b | 3 | −0.4 ± 13.8 | −1.8 ± 3.9 |
| | 4 | 9 | 9.7 ± 7.3 | 4.7 ± 2.5 |

Patients were administered 2.5, 5, 10, 15, or 30 mg doses of bardoxolone methyl once daily for 85 days.

8. Blood Pressure and QTcF in Healthy Volunteers

Intensive blood pressure monitoring was employed in a separate Thorough QT Study, which was conducted in healthy volunteers. In both bardoxolone methyl-treated groups, one given the therapeutic dose, 20 mg, which was also studied in BEACON, and one given the supratherapeutic dose of 80 mg, the change in blood pressure did not differ from changes observed in placebo-treated patients after 6 days of once daily administration. Bardoxolone methyl did not increase QTcF as assessed by placebo-corrected QTcF changes (ΔΔQTcF) after 6 days of treatment at 20 or 80 mg.

Bardoxolone methyl has also been tested in non-CKD disease settings. In early clinical studies of bardoxolone methyl in oncology patients (RTA 402-C-0501, RTA 402-C-0702), after 21 consecutive days of treatment at doses that ranged from 5 to 1300 mg/day (crystalline formulation), no mean change in blood pressure was observed across all treatment groups. Similarly, in a randomized, placebo-controlled study in patients with hepatic dysfunction (RTA 402-C-0701), 14 consecutive days of bardoxolone methyl treatment at doses of 5 and 25 mg/day (crystalline formulation) resulted in mean decreases in systolic and diastolic blood pressure (Table 16).

Collectively, these data suggest that bardoxolone methyl does not prolong the QT interval and does not cause blood pressure increases in patients who do not have baseline cardiovascular morbidity or stage 4 CKD.

TABLE 16
Changes from Baseline in Blood Pressure in Patients with
Hepatic Dysfunction Treated with Bardoxolone Methyl

| | | Mean ΔSBP ± SE (mmHg) | | Mean ΔDBP ± SE (mmHg) | |
|---|---|---|---|---|---|
| Dose | N | D 7 | D 14 | D 7 | D 14 |
| PBO | 4 | −10 ± 8.5 | −1.3 ± 5.5 | −4.0 ± 2.0 | 0.0 ± 3.1 |
| 5 mg | 6 | −12.8 ± 5.2 | −8.8 ± 5.1 | −2.0 ± 2.3 | −1.7 ± 3.2 |
| 25 mg | 6 | −11.5 ± 5.2 | −1.2 ± 3.6 | −4.0 ± 2.8 | −1.5 ± 4.1 |

9. Summary and Analysis of Heart Failure

Comparison of baseline characteristics of patients with heart failure events revealed that while the risk for heart failure was higher in the bardoxolone methyl-treated patients, both bardoxolone methyl-treated and placebo-treated patients with heart failure were more likely to have had a prior history of cardiovascular disease and heart failure and on average, had higher baseline ACR, BNP, and QTcF. Thus, development of heart failure in these patients was likely associated with traditional risk factors for heart failure. Additionally, many of the patients with heart failure were in subclinical heart failure prior to randomization, as indicated by their high baseline BNP levels.

As a surrogate of fluid retention after randomization, post-hoc analysis was performed on a subset of patients for whom BNP data were available, and increases were significantly greater in bardoxolone methyl-treated patients vs. placebo-treated patients at Week 24, with the BNP increases in bardoxolone methyl-treated patients directly correlated with baseline ACR. Urinary sodium excretion data from BEACON ABPM sub-study patients revealed a clinically meaningful reduction in urine volume and excretion of sodium at Week 4 relative to baseline in the bardoxolone methyl-treated patients only. In another study, urinary sodium levels and water excretion were reduced in stage 4 CKD patients but not in stage 3b CKD patients. Together, these data suggest that bardoxolone methyl differentially affects sodium and water handling, with retention of these more pronounced in patients with stage 4 CKD.

Consistent with this phenotype for fluid retention, post-hoc review of the narrative descriptions for heart failure events provided in hospital admission notes, together with anecdotal reports from investigators, indicates that heart failure events in bardoxolone methyl-treated patients were often preceded by rapid fluid weight gain and were not associated with acute decompensation of the kidneys or heart.

Blood pressure changes, indicative of overall volume status, were also increased in the bardoxolone methyl group relative to the placebo group as measured by standardized blood pressure cuff monitoring in BEACON. Pre-specified blood pressure analysis in healthy volunteer studies demonstrated no changes in either systolic or diastolic blood pressure. While the intent-to-treat (ITT) analyses of phase 2 CKD studies conducted with bardoxolone methyl showed no clear changes in blood pressure, post-hoc analyses of these studies suggest that increases in both systolic and diastolic blood pressure are dependent on CKD stage. Taken together, these data suggest that the effects of bardoxolone methyl treatment on blood pressure may be associated with CKD disease severity.

Thus, the urinary electrolyte, BNP, and blood pressure data collectively support that bardoxolone methyl treatment can differentially affect volume status, having no clinically detectable effect in healthy volunteers or early-stage CKD patients, while likely promoting fluid retention in patients with more advanced renal dysfunction and with traditional risk factors associated with heart failure at baseline. The increases in eGFR are likely due to glomerular effects whereas effects on sodium and water regulation are tubular in origin. As eGFR change was not correlated with heart failure, the data suggest that effects on eGFR and sodium and water regulation are anatomically and pharmacologically distinct.

The increased risk for heart failure and related adverse events with bardoxolone methyl treatment was not observed in prior studies (Table 17). However, since prior studies of bardoxolone methyl enrolled 10-fold fewer patients, the increased risk, if present, may have been undetectable. Moreover, BEACON limited enrollment to patients with stage 4 CKD, a population known to be at higher risk for cardiovascular events relative to patients with stage 3b CKD. Thus, the advanced nature of renal disease and significant cardiovascular risk burden of the BEACON population (manifested, among other markers, by low baseline eGFR, high baseline ACR, and high baseline BNP levels) were likely important factors in the observed pattern of cardiovascular events.

To examine further the relationship between key endpoints in BEACON and clinically meaningful thresholds of traditional risk factors of fluid overload, an additional post-hoc analysis was performed. Various eligibility criteria related to these risk factors were applied to exclude patients at most risk and explore the resulting outcomes from BEACON. Combinations of select criteria, including exclusion of patients with eGFR of 20 mL/min/1.73 m² or less, markedly elevated levels of proteinuria, and either age over 75 or BNP greater than 200 pg/mL abrogate the observed imbalances (Table 18). Applying these same criteria to SAEs also markedly improves or abrogates the noted imbalances (Table 19). Taken together, these findings suggest utility of these and other renal and cardiovascular risk markers in future selection criteria for clinical studies with bardoxolone methyl.

TABLE 17
Frequency of Treatment-Emergent Adverse Events Related to Heart Failure¹ by
Primary System Organ Class (SOC) Observed in Prior Chronic Kidney Disease Studies with
Bardoxolone Methyl

| | | | 0804 (BEAM) | | | | 0902 | | | | |
|---|---|---|---|---|---|---|---|---|---|---|---|
| | Study | | BARD (Crystalline) | | | | BARD (SDD) | | | | |
| | | Preferred | PBO | 25 mg | 75 mg | 150 mg | 2.5 mg | 5 mg | 10 mg | 15 mg | 30 mg |
| | SOC | Term | (N = 57) | (N = 57) | (N = 57) | (N = 56) | (N = 14) | (N = 25) | (N = 28) | (N = 50) | (N = 14) |
| AEs | Metab | Oedema | 3 (5) | 3 (5) | 1 (2) | 3 (5) | 0 | 0 | 0 | 0 | 1 (7) |
| | | peripheral | | | | | | | | | |
| | | Fluid overload | 0 | 3 (5) | 2 (4) | 0 | — | — | — | — | — |
| | Genrl | Oedema | 11 (19) | 11 (19) | 10 (18) | 11 (20) | 0 | 3 (12) | 5 (18) | 3 (6) | 3 (21) |
| | | peripheral | | | | | | | | | |
| | | Generalised | 0 | 2 (4) | 0 | 0 | — | — | — | — | — |
| | | oedema | | | | | | | | | |
| | Resp | Dyspnoea | 5 (9) | 2 (4) | 6 (11) | 4 (7) | 0 | 0 | 0 | 0 | 1 (7) |
| | | Dyspnoea | 0 | 1 (2) | 0 | 3 (5) | 1 (7) | 0 | 0 | 0 | 0 |
| | | exertional | | | | | | | | | |
| | | Orthopnoea | 1 (2) | 0 | 0 | 0 | — | — | — | — | — |
| | | Pulmonary | 0 | 0 | 1 (2) | 0 | — | — | — | — | — |
| | | oedema | | | | | | | | | |
| | Inv | Ejection | 0 | 1 (2) | 0 | 0 | — | — | — | — | — |
| | | fraction | | | | | | | | | |
| | | decreased | | | | | | | | | |
| | Card | Oedema | 1 (2) | 4 (7) | 3 (5) | 4 (7) | 0 | 0 | 1 (4) | 1 (2) | 0 |
| | | peripheral | | | | | | | | | |
| | | Cardiac failure | 3 (5) | 2 (4) | 3 (5) | 3 (5) | 0 | 0 | 1 (4) | 0 | 1 (7) |
| | | congestive | | | | | | | | | |
| | | Dyspnoea | 0 | 0 | 1 (2) | 0 | — | — | — | — | — |
| | | paroxysmal | | | | | | | | | |
| | | nocturnal | | | | | | | | | |
| SAEs | Card | Cardiac failure | 3 (5) | 2 (4) | 2 (4) | 2 (4) | 0 | 0 | 1 (4) | 0 | 1 (7) |
| | | congestive | | | | | | | | | |
| | Genrl | Oedema | 0 | 0 | 0 | 1 (2) | — | — | — | — | — |
| | | peripheral | | | | | | | | | |
| | Metab | Fluid overload | 0 | 1 (2) | 1 (2) | 0 | — | — | — | — | — |
| | Resp | Dyspnoea | 1 (2) | 0 | 0 | 0 | — | — | — | — | — |
| | | Pulmonary | 0 | 0 | 1 (2) | 0 | — | — | — | — | — |
| | | oedema | | | | | | | | | |

In 402-C-0804, patients were administered 25, 75, 150 mg of bardoxolone methyl (crystalline formulation) or placebo once daily for 52 weeks. In RTA402-C-0903, patients were administered 2.5, 5, 10, 15, or 30 mg doses of bardoxolone methyl (SDD formulation) once daily for 85 days.
1 Adverse events with preferred terms matching Standardized MedDRA Queries for cardiac failure outlined in the BEACON EAC Charter (Submission Serial 133, dated Feb. 2, 2012).

TABLE 18
Effect of Excluding Patients with Select Baseline Characteristics on Primary
Endpoints, Heart Failure, and All-Cause Mortality in BEACON

| | | | Eligibility Criteria (N) | | | | | | |
|---|---|---|---|---|---|---|---|---|---|
| | | | | | | | | BL | |
| | | | | No | | | | ACR ≤ 1000, | BL ACR ≤ 300, |
| | Observed | | BL | h/o | BL | BL | | eGFR > 20, | eGFR > 20, |
| Event | | N | BNP ≤ 200 | HF | ACR ≤ 1000 | eGFR > 20 | Age ≤ 75 | Age ≤ 75 | BNP ≤ 200 |
| Heart | BARD | 103 | 22 | 67 | 63 | 56 | 75 | 19 | 5 |
| Failure | PBO | 57 | 16 | 36 | 40 | 37 | 45 | 20 | 3 |
| All-Cause | BARD | 44 | 14 | 35 | 32 | 27 | 20 | 11 | 5 |
| Death | PBO | 31 | 8 | 24 | 21 | 18 | 23 | 11 | 4 |
| ESRD | BARD | 47 | 12 | 35 | 21 | 18 | 38 | 9 | 1 |
| | PBO | 55 | 22 | 44 | 27 | 14 | 46 | 6 | 1 |
| Randomized | BARD | 1088 | 559 | 922 | 798 | 735 | 786 | 368 | 209 |
| Patients | PBO | 1097 | 593 | 943 | 792 | 718 | 829 | 400 | 217 |

Post-hoc analysis of outcomes in BEACON. Observed totals for number of patients with heart failure, all-cause and cardiovascular deaths, and ESRD includes all events through last date of contact (ITT Population).

TABLE 19
Effect of Excluding Patients with Select Baseline Characteristics on Treatment-
Emergent Serious Adverse Events by Primary SOC in BEACON (ITT Population)

| | Primary SOC | | | | | |
|---|---|---|---|---|---|---|
| | | | BL ACR ≤ | | | |
| | | | 1000, | | BL ACR ≤ 300, | |
| | | | eGFR > 20, | | eGFR > 20, | |
| | All Patients | | Age ≤ 75 | | BNP ≤ 200 | |
| | PBO | BARD | PBO | BARD | PBO | BARD |
| Treatment | (N = 1097) | (N = 1088) | (N = 400) | (N = 368) | (N = 217) | (N = 209) |
| Blood and lymphatic system | 11 (1) | 20 (2) | 3 (<1) | 4 (<1) | 2 (<1) | 0 |
| disorders | | | | | | |
| Cardiac disorders | 84 (8) | 124 (11) | 32 (3) | 35 (3) | 10 (1) | 16 (1) |
| Ear and labyrinth disorders | 1 (<1) | 3 (<1) | 1 (<1) | 1 (<1) | 0 | 1 (<1) |
| Endocrine disorders | 1 (<1) | 1 (<1) | 1 (<1) | 1 (<1) | 1 (<1) | 1 (<1) |
| Eye disorders | 2 (<1) | 3 (<1) | 1 (<1) | 1 (<1) | 1 (<1) | 0 |
| Gastrointestinal disorders | 39 (4) | 46 (4) | 13 (1) | 10 (1) | 8 (1) | 7 (1) |
| General disorders and administration | 20 (2) | 29 (3) | 3 (<1) | 2 (<1) | 2 (<1) | 3 (<1) |
| site conditions | | | | | | |
| Hepatobiliary disorders | 8 (1) | 4 (<1) | 2 (<1) | 1 (<1) | 0 | 1 (<1) |
| Immune system disorders | 0 | 2 (<1) | 0 | 0 | 0 | 0 |
| Infections and infestations | 63 (6) | 79 (7) | 20 (2) | 20 (2) | 12 (1) | 9 (1) |
| Injury, poisoning and procedural | 17 (2) | 19 (2) | 3(<1) | 4 (<1) | 0 | 2 (<1) |
| complications | | | | | | |
| Investigations | 2 (<1) | 3 (<1) | 1 (<1) | 2 (<1) | 0 | 0 |
| Metabolism and nutrition disorders | 42 (4) | 51 (5) | 11 (1) | 14 (1) | 9 (1) | 5 (<1) |
| Musculoskeletal and connective | 13 (1) | 21 (2) | 6 (1) | 9 (1) | 3 (<1) | 6 (1) |
| tissue disorders | | | | | | |
| Neoplasms benign, malignant and | 10 (1) | 11 (1) | 6 (1) | 3 (<1) | 2 (<1) | 1 (<1) |
| unspecified | | | | | | |
| Nervous system disorders | 35 (3) | 37 (3) | 13 (1) | 6 (1) | 9 (1) | 4 (<1) |
| Psychiatric disorders | 3 (<1) | 3 (<1) | 1 (<1) | 2 (<1) | 1 (<1) | 1 (<1) |
| Renal and urinary disorders | 71 (6) | 52 (5) | 14 (1) | 9 (1) | 2 (<1) | 4 (<1) |
| Reproductive system and breast | 3 (<1) | 2 (<1) | 0 | 0 | 0 | 0 |
| disorders | | | | | | |
| Respiratory, thoracic and | 32 (3) | 43 (4) | 11 (1) | 15 (1) | 7 (1) | 6 (1) |
| mediastinal disorders | | | | | | |
| Skin and subcutaneous tissue | 1 (<1) | 4 (<1) | 1 (<1) | 1 (<1) | 1 (<1) | 1 (<1) |
| disorders | | | | | | |
| Surgical and medical procedures | 0 | 2 (<1) | 0 | 1 (<1) | 0 | 1 (<1) |
| Vascular disorders | 18 (2) | 20 (2) | 5 (<1) | 4 (<1) | 2 (<1) | 2 (<1) |

Post-hoc analyses of treatment-emergent serious adverse events in BEACON. Event totals include only SAEs with onset no more than 30 days after a patient's last dose of study drug.

D. Potential Mechanisms of Fluid Overload in BEACON

Data presented in prior sections suggest that bardoxolone methyl promotes fluid retention in a subset of patients who are at most risk of developing heart failure independent of drug administration. The data suggest that the effects are not associated with acute or chronic renal or cardiac toxicity. Therefore, a comprehensive list of well-established renal mechanisms that affect volume status (Table 20) was explored to determine if any of the etiologies matched the clinical phenotype observed with bardoxolone methyl.

Initial investigation focused on possible activation of the renin-angiotensin-aldosterone system. Activation of this pathway reduces serum potassium due to increased renal excretion. However, bardoxolone methyl did not affect serum potassium and slightly reduced urinary potassium in the BEACON sub-study (Table 10).

Another potential mechanism that was investigated was whether transtubular ion gradient changes may have resulted in sodium and consequent water resorption, since bardoxolone methyl affects serum magnesium and other electrolytes.

However, this mechanism also involves potassium regulation, and baseline serum magnesium did not appear to be associated with fluid retention or heart failure hospitalizations.

After other etiologies were excluded for reasons listed in Table 19, suppression of endothelin signaling was the primary remaining potential mechanism of volume regulation that was consistent with the bardoxolone methyl treatment effect in

BEACON. Therefore, an extensive investigation of modulation of the endothelin pathway as a potential explanation for fluid retention observed in the BEACON study was conducted.

TABLE 20
Established Renal Mechanisms Affecting Volume Status

| | Na⁺ | K⁺ | Effect on | |
|---|---|---|---|---|
| Mechanism | Retention | Retention | GFR | Comments |
| Bardoxolone Methyl | ↑ | None | ↑ | ↑ Na⁺ retention independent of K⁺ Stage 4 CKD patients, ↑ GFR |
| Endothelin | ↓ | None | ↓ | Suppression of endothelin fits BARD pattern |
| Endothelial Nitric | ↓ | None | ↑ | NO ↓ Na⁺ reabsorption and ↑ GFR |
| Oxide (NO) | | | | BARD ↑ both Na⁺ and GFR |
| | | | | BARD has been shown in vitro and in vivo to increase bioavailable |
| | | | | endothelial NO, but Na⁺ effect is likely independent of NO and GFR |
| | | | | changes |
| Antidiuretic | ↑ | ↑ | ↓ at ↑ levels | ADH ↑Na⁺ and K⁺ while ↓GFR |
| Hormone (ADH) | | | of ADH | BARD does not affect K⁺ and ↑ GFR |
| Transtubular ion | ↑ with | ↑ | No direct | Ion gradients have dual effect on Na⁺ and K⁺; Cl, HCO₃⁻ gradient often |
| gradients | ↑ GFR | | effect | generated as HCO₃⁻ absorption dependent on Na⁺ absorption |
| | | | | BARD does not affect K⁺ or HCO₃⁻ |
| Renin-Angiotensin- | ↑ | ↓ | ↑ | RAAS signaling ↑ K urinary excretion and ↓ serum levels |
| Aldosterone (RAAS) | | | | BARD does not affect K⁺ levels and has been shown to ↓ AII levels in |
| | | | | CKD patients and suppress AII signaling in vitro and in vivo |
| Pressure Natriuresis | ↓ | ↓ | Slight ↑ | Volume expansion leads to ↑ medullary plasma flow and ↓ |
| | | | | hypertonicity; ↓ water absorption in the loop of Henle with ↓ of Na⁺ |
| | | | | and K⁺ |
| | | | | BARD-mediated magnitude of volume expansion unlikely sufficient |
| | | | | to promote this effect; BARD ↑ Na⁺ and does not affect K⁺ |
| Prostaglandins (PGE₂, | ↓ | Slight ↓ | ↑ | PGs ↑GFR and ↑Na⁺ urine excretion |
| PGI₂) | | | | BARD ↑ Na⁺ retention, not excretion |
| Natriuretic peptides | ↓ | Slight ↓ | ↑ | Natriuretic peptides have divergent effects on Na⁺ and GFR with |
| | | | | slight effect on K⁺ |
| | | | | BNP and other natriuretic peptides ↑ Na⁺ urine excretion |
| | | | | BARD ↑ Na⁺ retention, not excretion |
| | | | | BARD does no interfere with natriuretic peptides, as GFR would |
| | | | | likely ↓ |
| Peritubular factors | ↑ with | ↑ with | None | Na⁺ and K⁺ move with GFR |
| | ↑ GFR | ↑ GFR | | BARD does not affect K⁺ |

Mechanisms and characteristics of fluid retention.

1. Modulation of the Endothelin System

The most directly analogous clinical data for comparison of the effects of known endothelin pathway modulators to the BEACON study are those with the endothelin receptor antagonist (ERA) avosentan. Avosentan was studied in stage 3-4 CKD patients with diabetic nephropathy in the ASCEND study, a large outcomes study to assess the time to first doubling of serum creatinine, ESRD, or death (Mann et al., 2010). While the baseline eGFR in this study was slightly above the mean baseline eGFR in BEACON, patients in the ASCEND study had a mean ACR that was approximately seven-fold higher than BEACON (Table 21). Therefore, the overall cardiovascular risk profile was likely similar between the two studies.

As in BEACON, the ASCEND study was terminated prematurely due to an early imbalance in heart failure hospitalization and fluid overload events. Importantly, avosentan-induced fluid overload-related adverse events, including serious and non-serious, were increased only within the first month of treatment.

Examination of key endpoints in the ASCEND study reveals an approximate three-fold increase in risk of congestive heart failure (CHF) with a modest, non-significant increase in death. Additionally, a small, numerical reduction in ESRD events was also observed. The BEACON study demonstrated similar findings, albeit with a lower incidence of heart failure events. Nonetheless, the two studies showed striking similarities in clinical presentation and timing of heart failure, as well as influences on other key endpoints (Table 22).

TABLE 21
Select Demographic and Baseline Characteristics for Patients in ASCEND* and BEACON (ITT Population)

| | ASCEND | | | BEACON | |
|---|---|---|---|---|---|
| | PBO | Avosentan 25 mg | Avosentan 50 mg | PBO | BARD 20 mg |
| BL Characteristic | (N = 459) | (N = 455) | (N = 478) | (N = 1097) | (N = 1088) |
| Age | 61 ± 9 | 61 ± 9 | 61 ± 9 | 68 ± 9 | 69 ± 10 |
| History of CHF (% of patients) | 13.5% | 14.5% | 14.4% | 15% | 14% |
| Systolic Blood Pressure (mmHg) | 135 ± 15 | 137 ± 14 | 137 ± 14 | 140 ± 12 | 140 ± 12 |
| BMI (kg/m²) | 30 ± 6 | 30 ± 6 | 30 ± 7 | 34 ± 7 | 34 ± 7 |
| eGFR (mL/min/1.73 m²) | 33 ± 11 | 34 ± 11 | 33 ± 11 | 22 ± 5 | 22 + 4 |
| Median ACR (mg/g) | 1540 | 1416 | 1474 | 221 | 210 |
| ACEi/ARB (% of patients) | 100% | 100% | 100% | 84% | 85% |
| Diuretics (% of patients) | 65% | 64% | 65% | 64% | 64% |

*Results from a randomized, double-blind, placebo-controlled trial of 1392 patients with type 2 diabetes and overt nephropathy receiving avosentan (25 or 50 mg) or placebo in addition to continued angiotensin-converting enzyme inhibition and/or angiotensin receptor blockade (ASCEND).

TABLE 22
Occurrence of Death, End Stage Renal Disease, or Heart
Failure in ASCEND and BEACON (ITT Population)

| | ASCEND | | | BEACON | |
|---|---|---|---|---|---|
| | PBO | Avosentan 25 mg | Avosentan 50 mg | PBO | BARD 20 mg |
| Event | (N = 459) | (N = 455) | (N = 478) | (N = 1097) | (N = 1088) |
| CHF | 2.2% | 5.9%* | 6.1%* | 5.0% | 8.8%* |
| Death | 2.6% | 3.6% | 4.6% | 2.8% | 4.0% |
| ESRD | 6.5% | 4.4% | 5.0% | 4.6% | 4.0% |

Occurrence of adjudicated CHF, death, and ESRD events in ASCEND and BEACON. In ASCEND, for an event to be qualified as CHF, the patient had to have typical signs and/or symptoms of heart failure and receive new therapy for CHF and be admitted to the hospital for at least 24 hours; ESRD was defined as need for dialysis or renal transplantation or an eGFR <15 mL/mm/1.73 m². Percentages for BEACON include all CHF and ESRD events through last date of contact and total number of deaths at the time of database lock (Mar. 21, 2013). ESRD in BEACON was defined as need for chronic dialysis, renal transplantation, or renal death; additional details and definitions for heart failure are outlined in the BEACON EAC Charter.
*p < 0.05 vs. placebo.

2. Mechanism of Endothelin Receptor Antagonist-Induced Fluid Overload

The role of endothelin in fluid overload has been extensively investigated. Through the use of knock-out models in mice, investigators have demonstrated that acute disruption of the endothelin pathway followed by a salt challenge promotes fluid overload. Specific knock-out of endothelin-1 (ET-1), endothelin receptor type A (ETA), endothelin receptor type B (ETB), or the combination of ETA and ETB have all been shown to promote fluid overload in animals with a clinical phenotype consistent with ERA-mediated fluid overload in patients. These effects are caused by acute activation of the epithelial sodium channel (ENaC), which is expressed in the collecting ducts of the kidney, where it reabsorbs sodium and promotes fluid retention (Vachiery and Davenport, 2009).

3. Relationship between Plasma and Urinary Endothelin-1 in Humans

An assessment of plasma and urinary levels of endothelin-1 (ET-1) in humans with eGFR values ranging from stage 5 CKD to supra-normal (8 to 131 mL/min/1.73 m²) has been previously reported (Dhaun et al., 2009). Plasma levels significantly and inversely correlated with eGFR, but due to the modest slope of the curve, meaningful differences of ET-1 were not readily apparent across the large eGFR range assessed. As a surrogate for kidney production of ET-1, the organ where the most ET-1 is produced, fractional excretion of ET-1 was calculated by assessing the plasma and urinary levels of ET-1. From eGFRs >100 to approximately 30 mL/min/1.73 m², urinary levels were relatively unchanged. However, ET-1 levels appear to increase exponentially with decreasing eGFR in patients with stage 4 and 5 CKD. These data suggest that renal ET-1 is primarily dysregulated in patients with advanced (stage 4 and 5) CKD. Based on these published data, the inventors hypothesized that the differential effects on fluid handling by bardoxolone methyl, if due to endothelin modulation, could be due to the disparate endogenous production of ET-1 in the kidney, which is meaningfully increased in stage 4 and 5 CKD patients.

4. Bardoxolone Methyl Modulates Endothelin Signaling

As described above, bardoxolone methyl reduces ET-1 expression in human cell lines, including mesangial cells found in the kidney as well as endothelial cell. Furthermore, in vitro and in vivo data suggest that bardoxolone methyl and analogs modulate the endothelin pathway to promote a vasodilatory phenotype by suppressing the vasoconstrictive ETA receptor and restoring normal levels of the vasodilatory ETB receptor. Thus, the potent activation of Nrf2-related genes with bardoxolone methyl is associated with suppression of pathological endothelin signaling and facilitates vasodilation by modulating expression of ET receptors.

E. Rationale for BEACON Termination

1. Adjudicated Heart Failure

Hospitalizations for heart failure or death due to heart failure were among the cardiovascular events adjudicated by the EAC. An imbalance in adjudicated heart failure and related events was the major finding that contributed to the early termination of BEACON. Additionally, heart failure-related AEs, such as edema, contributed to a higher discontinuation rate than expected. The overall imbalance in time-to-first adjudicated heart failure appeared to result from the large contribution of events occurring within the first three to four weeks after initiation of treatment. The Kaplan-Meyer analysis shows that after this initial period the event rates between the treatment arms appear to maintain parallel trajectories. The pattern reflected an acute, physiologic effect that precipitated hospitalization for heart failure versus a cumulative toxic effect.

2. Mortality

At the time of the termination of the study, more deaths had occurred in the bardoxolone methyl group than in the placebo group, and the relationship between mortality and heart failure was unclear. A majority of the fatal outcomes (49 of the 75 deaths) occurring prior to clinical database lock (Mar. 4, 2013) were confirmed as being cardiovascular in nature (29 bardoxolone methyl patients vs. 20 placebo patients). Most of the cardiovascular deaths were classified as “cardiac death—not otherwise specified,” based on pre-specified definitions outlined in the BEACON EAC charter. On final analysis, the Kaplan-Meier analysis for overall survival showed no apparent separation until approximately Week 24. There were three fatal heart failure events, all in bardoxolone methyl-treated patients. In addition, as reflected in Table 16, the percentage of deaths occurring in patients that were over 75 years old was higher in bardoxolone methyl-treated patients compared to placebo-treated patients. Notably, if patients over 75 years old are excluded, the numbers of fatal events in the bardoxolone methyl arm compared to the placebo arm are 20 and 23, respectively.

3. Summary of Other Safety Data from BEACON

In addition to the effects of bardoxolone methyl treatment on eGFR and renal SAEs, the number of hepatobiliary SAEs was reduced in the bardoxolone methyl group relative to the placebo group (4 versus 8, respectively; Table 5), and no Hy's Law cases were observed. The number of neoplasm-related SAEs was also balanced across both groups. Lastly, bardoxolone methyl treatment was not associated with QTc prolongation, as assessed by ECG assessments at Week 24 (Table 23).

TABLE 23
Change from Baseline in QTcF at Week 24 in Bardoxolone Methyl
versus Placebo Patients in BEACON (Safety Population)

| Timepoint/ | Observed | | Change from baseline | |
|---|---|---|---|---|
| QTcF | | Bardoxolone | | Bardoxolone |
| interval | Placebo | methyl | Placebo | methyl |
| (msec) | N = 1093 | N = 1092 | N = 1093 | N = 1092 |
| n | 719 | 639 | 719 | 637 |
| Mean (SD) | 428.8 (29.2) | 425.8 (26.5) | 3.6 (16.4) | −0.9 (19.2) |
| Range (min, | 362, 559 | 355, 518 | −59, 82 | −88, 69 |
| max) | | | | |
| Quartiles | 408, 426, 445 | 407, 425, 443 | −7, 3, 13 | −13, −1, 10 |
| (25th, | | | | |
| median, | | | | |
| 75th) | | | | |

Data includes only ECG assessments collected on or before a patient's last dose of study drug. Visits are derived relative to a patient's first dose of study drug.

F. BEACON Conclusions

In summary, interrogation of data from studies conducted with bardoxolone methyl revealed that the drug can differentially regulate fluid retention, with no clinically detectable effect in healthy volunteers or early-stage CKD patients, while likely pharmacologically promoting fluid retention in patients with advanced renal dysfunction. Since the development of heart failure in both bardoxolone methyl- and placebo-treated patients was associated with traditional risk factors for heart failure, this pharmacological effect in patients with baseline cardiac dysfunction may explain the increased risk for heart failure with bardoxolone methyl treatment in BEACON. These data suggest that decreasing the overall risk for heart failure in future clinical studies by selecting a patient population with lower baseline risk for heart failure should avoid increases in heart failure associated with bardoxolone methyl treatment. Importantly, the available data show that fluid overload in BEACON was not caused by a direct renal or cardiac toxicity. The clinical phenotype of fluid overload is similar to that observed with ERAs in advanced CKD patients, and preclinical data demonstrate that bardoxolone methyl modulates the endothelin pathway. As acute disruption of the endothelin pathway in advanced CKD patients is known to activate a specific sodium channel (ENaC) that can promote acute sodium and volume retention (Schneider, 2007), these mechanistic data, along with the clinical profile of bardoxolone methyl patients with heart failure, provide a reasonable hypothesis to the mechanism of fluid retention in BEACON. Because compromised renal function may be an important factor that contributes to a patient's inability to compensate for short-term fluid overload, and because relatively limited numbers of patients with earlier stages of CKD have been treated to date, exclusion of patients with CKD (e.g., patients with an eGFR <60) from treatment with BARD and other AIMs may be prudent and may be included as an element of the present invention.

### VI. Diagnostic Tests

A. Measurement of B-type Natriuretic Peptide (BNP) Levels

B-type natriuretic peptide (BNP) is a 32-amino acid neurohormone that is synthesized in the ventricular myocardium and released into circulation in response to ventricular dilation and pressure overload. The functions of BNP include natriuresis, vasodilation, inhibition of the renin-angiotensin-aldosterone axis, and inhibition of sympathetic nerve activity. The plasma concentration of BNP is elevated among patients with congestive heart failure (CHF), and increases in proportion to the degree of left ventricular dysfunction and the severity of CHF symptoms.

Numerous methods and devices are well known to the skilled artisan for measuring BNP levels in patient samples, including serum and plasma. With regard to polypeptides, such as BNP, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. Nos. 5,631,170 and 5,955,377. In a specific example, B-type natriuretic peptide (BNP) levels may be determined by the following method(s): protein immunoassays as described in US Patent Publication 2011/0201130, which is incorporated by reference in its entirety herein. Furthermore, a number of commercially available methods exist (e.g., Rawlins et al., 2005, which is incorporated herein by reference in its entirety).

B. Measurement of Albumin/Creatinine Ratio (ACR)

Conventionally, proteinuria is diagnosed by a simple dipstick test. Traditionally, dipstick protein tests are quantified by measuring the total quantity of protein in a 24-hour urine collection test.

Alternatively the concentration of protein in the urine may be compared to the creatinine level in a spot urine sample. This is termed the protein/creatinine ratio (PCR). The UK Chronic Kidney Disease Guidelines (2005; which are incorporated herein by reference in their entirety) states PCR is a better test than 24-hour urinary protein measurement. Proteinuria is defined as a protein/creatinine ratio greater than 45 mg/mmol (which is equivalent to albumin/creatinine ratio of greater than 30 mg/mmol or approximately 300 mg/g as defined by dipstick proteinuria of 3+) with very high levels of proteinuria being for a PCR greater than 100 mg/mmol.

Protein dipstick measurements should not be confused with the amount of protein detected on a test for microalbuminuria, which denotes values for protein for urine in mg/day versus urine protein dipstick values which denote values for protein in mg/dL. That is, there is a basal level of proteinuria that can occur below 30 mg/day which is considered non-pathological. Values between 30-300 mg/day are termed microalbuminuria which is considered pathologic. Urine protein lab values for microalbumin of >30 mg/day correspond to a detection level within the “trace” to “1+” range of a urine dipstick protein assay. Therefore, positive indication of any protein detected on a urine dipstick assay obviates any need to perform a urine microalbumin test as the upper limit for microalbuminuria has already been exceeded.

C. Measurement of Estimated Glomerular Filtration Rate (eGFR)

A number of formulae have been devised to estimate GFR values on the basis of serum creatinine levels. A commonly used surrogate marker for estimate of creatinine clearance (eC_{Cr}) is the Cockcroft-Gault (CG) formula, which in turn estimates GFR in mL/min. It employs serum creatinine measurements and a patient's weight to predict the creatinine clearance. The formula, as originally published, is:

$$eCCr = \frac{( 140 - Age ) \times Mass ( in kg )}{72 \times Serum creatinine ( in \frac{mg}{dL} )}$$

This formula expects weight to be measured in kilograms and creatinine to be measured in mg/dL, as is standard in the USA. The resulting value is multiplied by a constant of 0.85 if the patient is female. This formula is useful because the calculations are simple and can often be performed without the aid of a calculator.

When serum creatinine is measured in μmon, then:

$$eCCr = \frac{( 140 - Age ) \times Mass ( in kg ) \times Constant}{Serum creatinine ( in \frac{µmol}{L} )}$$

where Constant is 1.23 for men and 1.04 for women.

One interesting feature of the Cockcroft and Gault equation is that it shows how dependent the estimation of C_{Cr} is based on age. The age term is (140−age). This means that a 20-year-old person (140−20=120) will have twice the creatinine clearance as an 80-year-old (140−80=60) for the same level of serum creatinine. The CG equation assumes that a woman will have a 15% lower creatinine clearance than a man at the same level of serum creatinine.

Alternatively, eGFR values may be calculated using the Modification of Diet in Renal Disease (MDRD) formula. The 4-variable formula is as follows:

eGFR=175×Standardized serum creatinine^{-1.154}×Age^{-0.203}×C

where C is 1.212 if the patient is a black male, 0.899 if the patient is a black female, and 0.742 if the patient is a non-black female. Serum creatinine values are based on the IDMS-traceable creatinine determination (see below).

Chronic kidney disease is defined as a GFR less than 60 mL/min/1.73 m² that is present for three or more months.

D. Measurement of Serum Creatinine Levels

A serum creatinine test measures the level of creatinine in the blood and provides an estimate glomerular filtration rate. Serum creatinine values in the BEACON and BEAM trials were based on the isotope dilution mass spectrometry (IDMS)-traceable creatinine determinations. Other commonly used creatinine assay methodologies include (1) alkaline picrate methods (e.g., Jaffe method [classic] and compensated [modified] Jaffe methods), (2) enzymatic methods, (3) high-performance liquid chromatography, (4) gas chromatography, and (5) liquid chromatography. The IDMS method is widely considered to be the most accurate assay (Peake and Whiting, 2006, which is incorporated herein by reference in its entirety).

### VII. Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

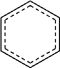

covers, for example,

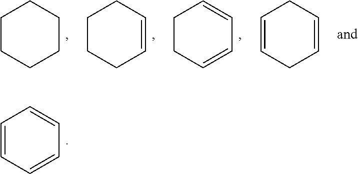

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

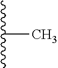

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

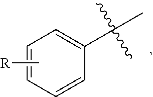

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

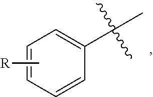

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “Cn” defines the maximum number (n) of carbon atoms that can be in the group/class, with the minimum number as small as possible for the group/class in question. For example, it is understood that the minimum number of carbon atoms in the groups “alkyl_{(C≤8)}”, “cycloalkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8})” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group refers to a planar unsaturated ring of atoms with 4n+2 electrons in a fully conjugated cyclic π system.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above. When any of these terms is used with the “substituted” modifier, one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

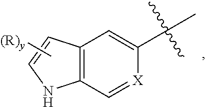

is a non-limiting example of cycloalkanediyl group. A “cycloalkane” refers to the class of compounds having the formula H—R, wherein R is cycloalkyl as this term is defined above. When any of these terms is used with the “substituted” modifier, one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂CCCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” when used without the “substituted” modifier refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

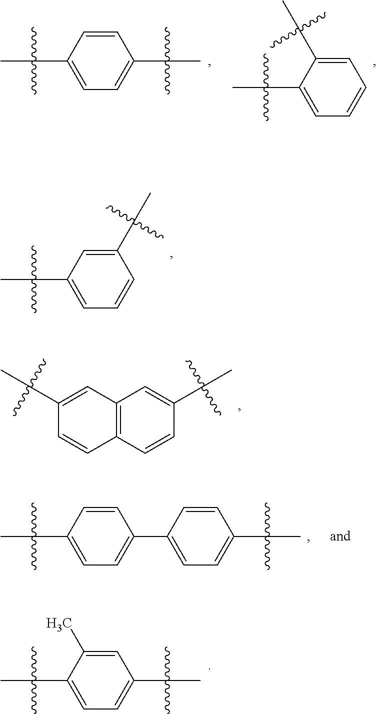

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms is nitrogen, oxygen or sulfur, and wherein the heteroaryl group consists of no atoms other than carbon, hydrogen, aromatic nitrogen, aromatic oxygen and aromatic sulfur. If more than one ring is present, the rings may be fused or unfused. Unfused rings are connected with a covalent bond. As used herein, the term heteroaryl does not preclude the presence of one or more alkyl or aryl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms is nitrogen, oxygen or sulfur, and wherein the heterocycloalkyl group consists of no atoms other than carbon, hydrogen, nitrogen, oxygen and sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier, one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, “alkyl sulfonylamino”, or “cycloalkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, alkylsulfonyl, and cycloalkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. When any of these terms is used with the “substituted” modifier, one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The terms “alkylsulfonyl” and “alkylsulfinyl” when used without the “substituted” modifier refers to the groups —S(O)₂R and —S(O)R, respectively, in which R is an alkyl, as that term is defined above. The terms “cycloalkylsulfonyl”, “alkenylsulfonyl”, “alkynylsulfonyl”, “aryl sulfonyl”, “aralkyl sulfonyl”, “heteroarylsulfonyl”, and “heterocycloalkylsulfonyl” are defined in an analogous manner. When any of these terms is used with the “substituted” modifier, one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients. When used in the context of X-ray powder diffraction, the term “about” is used to indicate a value of ±0.2 °2θ from the reported value, preferably a value of ±0.1 °2θ from the reported value. When used in the context of differential scanning calorimetry or glass transition temperatures, the term “about” is used to indicate a value of ±10° C. relative to the maximum of the peak, preferably a value of ±2° C. relative to the maximum of the peak. When used in other contexts, the term “about” is used to indicate a value of ±10% of the reported value, preferably a value of ±5% of the reported value. It is to be understood that, whenever the term “about” is used, a specific reference to the exact numerical value indicated is also included.

An “active ingredient” (AI) (also referred to as an active compound, active substance, active agent, pharmaceutical agent, agent, biologically active molecule, or a therapeutic compound) is the ingredient in a pharmaceutical drug or a pesticide that is biologically active. The similar terms active pharmaceutical ingredient (API) and bulk active are also used in medicine, and the term active substance may be used for pesticide formulations.

As used herein, average molecular weight refers to the weight average molecular weight (Mw) as determined by static light scattering.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or a subject with a compound means that amount of the compound which, when administered to the patient or the subject for treating or preventing a disease, is an amount sufficient to effect the treatment or prevention of the disease. In a particular embodiment, a measure of an effective treatment is a reduction in the concentration of protein in the urine to less than 300 mg/dL. In a preferred embodiment, the therapy is sufficient to reduce the concentration of protein in the urine to less than 100 mg/dL or a more preferred embodiment, less than 30 mg/dL. When the presence of blood is used as a marker of therapeutic effectiveness, an effective therapy results in the absence of macroscopic blood in the urine while microscopic blood may still be present. In a preferred embodiment, an effective therapy results in the absence of any blood including microscopic blood which would only be visible using a microscope or in an urinalysis. Finally, an effective therapy would result in an improvement in the glomerular filtration rate. Glomerular filtration rate can be estimated using a variety of different methods using creatinine including the Cockcroft-Gault formula, the Modification of Diet in Renal Disease (MDRD) formula, the Chronic Kidney Disease Epidemiology Collaboration (CKD-EPI) formula, the Mayo Quadratic formula, or the Schwartz formula. In general, the Schwartz formula may be used for children below the age of 12. These methods are further elaborated on in the sections above and in the Examples below. For example, an effective treatment may result a glomerular filtration rate (or an estimated glomerular filtration rate) of greater than 60 mL/min/1.73 m². More preferably, the effective treatment may result in an glomerular filtration rate of greater than 90 mL/min/1.73 m².

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Non-limiting examples of such salts include acid addition salts formed with inorganic acids such as hydrochloric acid, hydrobromic acid, sulfuric acid, nitric acid, and phosphoric acid; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, and trim ethyl acetic acid. Pharmaceutically acceptable salts al so include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Non-limiting examples of acceptable organic bases include ethanolamine, diethanolamine, triethanolamine, tromethamine, and N-methylglucamine. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical agent, pharmaceutical preparation, pharmaceutical composition, pharmaceutical formulation, pharmaceutical product, medicinal product, medicine, medication, medicament, or simply a drug) is a drug used to diagnose, cure, treat, or prevent disease. An active ingredient (AI) (defined above) is the ingredient in a pharmaceutical drug or a pesticide that is biologically active. The similar terms active pharmaceutical ingredient (API) and bulk active are also used in medicine, and the term active substance may be used for pesticide formulations. Some medications and pesticide products may contain more than one active ingredient. In contrast with the active ingredients, the inactive ingredients are usually called excipients (defined above) in pharmaceutical contexts.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-13-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—A Phase II/III, Randomized, Double-Blind, Placebo-Controlled Trial of the Effects of Bardoxolone Methyl in Patients Infected with SARS-Corona Virus-2 (COVID-19)

This multi-center, double-blind, placebo-controlled, randomized Phase 2/3 trial will study the safety, tolerability, and efficacy of bardoxolone methyl in approximately 400-440 patients hospitalized with confirmed COVID-19. Bardoxolone methyl or placebo will be administered for the duration of hospitalization (until recovery), for up to a maximum of 29 days. A final follow-up visit will occur 60 days after randomization. Total duration of study participation is estimated to be approximately 2 months.

Patients will be randomized using permuted block randomization in a 1:1 fashion to either once-daily administration of bardoxolone methyl (20 mg) or matching placebo. In some cases, the patients may be treated using a dose titration. The dose titration may be, for example, from 5 to 10 to 20 to 30 mg or from 10 to 20 to 30 mg. In any case, the dose titration may be capped at 20 mg. Patients may be stratified by age (≤50, 50 to 70, or ≥70 years of age). Randomization will be stratified by study center and invasive mechanical ventilation (i.e., mechanical ventilation with endotracheal intubation) use at baseline (yes or no). Randomization will be performed using an interactive web response system (IWRS). Following randomization on Day 1, patients will be assessed while hospitalized on Days 3, 5, 8, 11, 15, 22, and 29. Assessments will include clinical status assessments, vital sign measurements, clinical chemistry collection, and adverse event collection. Patients that recover prior to Day 29 will complete an end-of-treatment visit. Patients will have an in-person follow-up on Day 29, regardless of treatment adherence and recovery status prior to Day 29, and a safety follow-up 60 days after randomization for clinical status assessments, vital sign measurements, clinical chemistry collection, and adverse event collection. Follow-up in-person visits are preferred but recognizing quarantine and other factors may limit the subject's ability to return to the site for the visit. In this case, the visit may be performed by phone.

Enrollment of patients ≥70 years of age may be limited (e.g., comprise no more than 10% of all randomized patients), pending safety review by the DSMB and executive committee. Treatment will be administered for the duration of hospitalization (until recovery), with an expected duration of 10 days. For patients who are still hospitalized for more than 10 days, treatment can be continued for a maximum treatment duration of 29 days. Dose de-escalation (down to 10 mg) is permitted during the study if indicated clinically. Once a patient's dose has been reduced, dose re-escalation back to a higher dose is permitted.

The Phase 2 portion of the trial will include approximately 40 patients and is primarily designed to provide an initial assessment of the safety (e.g., frequency, intensity, and relationship to study drug of serious adverse events [including unexpected deaths], change from baseline in vital sign and laboratory assessments) of bardoxolone methyl in COVID-19 patients when compared with matching placebo. The phase 2 primary endpoint will be assessed during the treatment phase, defined as while hospitalized and up to Day 15.

Enrollment of the phase 3 cohort will initiate after safety and proof of concept has been confirmed in the Phase 2 cohort. The Phase 3 portion of the trial will include approximately 360-400 additional patients, and is primarily designed to (1) determine whether bardoxolone methyl in COVID-19 patients increases the probability of recovery (defined as alive, free of respiratory failure [e.g., need for non-invasive or invasive mechanical ventilation, high flow oxygen, or ECMO], and free of renal replacement therapy [RRT]) (WHO ordinal scale score ≤5 and no RRT) within 29 days when compared with matching placebo and (2) assess the safety of bardoxolone methyl in COVID-19 patients when compared with matching placebo. The phase 3 primary endpoint will be assessed during the treatment phase, defined as while hospitalized and up to Day 29. A secondary objective is to assess the effect of bardoxolone methyl on other endpoints, including renal function (e.g., change from baseline in eGFR up to Day 29 (or end of treatment)) and number of mechanical ventilation-free days while hospitalized up to Day 29, when compared with matching placebo.

Additional exploratory efficacy endpoints include:
Number of renal replacement therapy (RRT)-free days while hospitalized up to Day 29
Time to recovery up to Day 29
Recovery at Day 29
Recovery at Day 60
Proportion of subjects experiencing a deterioration from baseline (as defined by a 1-point worsening) to end of treatment or Day 29 (whichever comes first) in clinical status using an 11-category ordinal scale:
   0—Uninfected; no viral RNA detected
   1—Asymptomatic; viral RNA detected
   2—Symptomatic; Independent
   3—Symptomatic; assistance needed
   4—Hospitalized; no oxygen therapy
   5—Hospitalized; oxygen by mask or nasal prongs
   6—Hospitalized; oxygen by NIV or High flow
   7—Intubation & Mechanical ventilation; pO₂/FIO₂>1=150 or SpO₂/FIO₂>1=200
   8—Mechanical ventilation pO₂/FIO₂<150 (SpO₂/FIO₂<200) or vasopressors
   9—Mechanical ventilation pO₂/FIO₂<150 and vasopressors, dialysis or ECMO
   10— Death
All-cause mortality
Change from baseline in PaO2/FiO2 (while hospitalized on Day 5, 8, 15, and end of treatment)
Change from baseline in D-dimer, C-reactive protein (CRP), LDH, troponin and cytokine levels (while hospitalized on Day 5, 8, 15, and end of treatment)

Dosing: Several dose-ranging studies which have been conducted with bardoxolone methyl demonstrated that changes in eGFR are dose-dependent. The 20 mg bardoxolone methyl dose used in the present study provides near-optimal pharmacological activity and efficacy while also minimizing potential tolerability issues. Furthermore, most of the available clinical safety and efficacy data for bardoxolone methyl has been collected with the 20 mg dose.

Patients will be administered study drug orally once a day beginning on Day 1 while the patient remains hospitalized. Each dose of study drug should be administered at approximately the same time each day, preferably in the morning. Study drug will be administered at the time points listed in Table 24. Patients unable to receive oral medications (e.g., due to intubation and/or mechanical ventilation) may receive the contents of the capsule through a nasogastric or orogastric tube flushed with water. A vomited dose must not be replaced. A double dose (e.g., missed dose from previous day and dose for current day) must not be taken.

With regard to patient inclusion criteria for the study, an individual must meet all of the following criteria:
1. Laboratory-confirmed COVID-19 infection as determined by polymerase chain reaction (PCR)
2. Hospitalized patients that meets one of the following conditions:
   (i) Radiographic infiltrates by imaging (chest x-ray, CT scan, etc.); OR
   (ii) At rest, blood oxygen saturation ≤94%; OR
   (iii) Require supplemental oxygen; OR
   (iv) Requiring non-invasive ventilation; OR
   (v) Requiring mechanical ventilation for up to 2 days.
3. Age ≥18 years. Enrollment of patients ≥70 years of age may be limited (e.g., comprise no more than 10% of all randomized patients).
4. Participant or legally authorized representative is willing to give informed consent.

With regard to patient exclusion criteria for the study, all patients with any of the following conditions or characteristics will be excluded from the study:
1. Intubated and on mechanical ventilation (invasive) for three or more days at the time of randomization
2. Known left ventricular ejection fraction (LVEF)<40% or prior hospitalization for heart failure
3. Cardiac arrest
4. Shock
5. Uncontrolled bacterial, fungal, or non-COVID viral infection
6. History of eGFR <15 ml/min/1.73 m² or requiring dialysis
7. ALT or AST >5×ULN
8. History of cirrhosis, chronic active hepatitis or severe hepatic disease
9. Pregnant or lactating women
10. Enrolled in other trial of unapproved therapies, unless approved by trial Principal Investigator. In general, co-enrollment will be permitted unless there are safety concerns, mechanistic incompatibility or inability to adjudicate serious adverse events. Concomitant use with strong CYP3A4 inhibitors is prohibited. If a strong CYP3A4 inhibitor is medically necessary, study drug should be temporarily discontinued. Concomitant study drug use with moderate CYP3A4 inhibitors should be avoided whenever possible, and switching to an alternative agent should be considered.
11. If in the opinion of the clinical team, progression to death is imminent and inevitable within the next 24 hours, irrespective of the provision of treatments

With regard to safety endpoints, the following safety endpoints will be assessed during the study: (1) All adverse events that are serious, unexpected, and have a reasonable possibility of having been related to the study drug; and (2) All unexpected deaths.

An adverse event (AE) is any symptom, sign, illness or experience that develops or worsens in severity during the course of the study. Intercurrent illnesses or injuries should be regarded as adverse events. Abnormal results of diagnostic procedures are considered to be adverse events if the abnormality: results in study withdrawal; is associated with a serious adverse event; is associated with clinical signs or symptoms; leads to additional treatment or to further diagnostic tests; or is considered by the investigator to be of clinical significance.

Adverse events are classified as serious or non-serious. A serious adverse event is any AE that is: fatal; life-threatening; requires or prolongs hospital stay; results in persistent or significant disability or incapacity; a congenital anomaly or birth defect; or an important medical event. Important medical events are those that may not be immediately life threatening, but are clearly of major clinical significance. They may jeopardize the subject, and may require intervention to prevent one of the other serious outcomes noted above. For example, drug overdose or abuse, a seizure that did not result in in-patient hospitalization, or intensive treatment of bronchospasm in an emergency department would typically be considered serious. All adverse events that do not meet any of the criteria for serious should be regarded as non-serious adverse events.

Severity for each adverse event will be graded using the NCI Common Terminology Criteria for Adverse Events (CTCAE) system. All adverse events will be recorded:
Grade 1: Mild; asymptomatic or mild symptoms; clinical or diagnostic observations only; intervention not indicated.
Grade 2: Moderate; minimal, local or noninvasive intervention indicated; limiting age-appropriate instrumental activities of daily living.
Grade 3: Severe or medically significant but not immediately life-threatening; hospitalization or prolongation of hospitalization indicated; disabling; limiting self care activities of daily living.
Grade 4: Life-threatening consequences; urgent intervention indicated.
Grade 5: Death related to AE.

For all collected AEs, the clinician who examines and evaluates the participant will determine the AE's causality based on temporal relationship and his/her clinical judgment. The degree of certainty about causality will be graded using the categories below.
Definitely Related—The event follows: (a) a reasonable, temporal sequence from study drug or a study procedure; and (b) cannot be explained by the known characteristics of the participant's clinical state or other therapies; and (c) evaluation of the participant's clinical state indicates to the investigator that the experience is definitely related to study procedures.
Probably Related—The event should be assessed following the same criteria for “Definitely Related”. If in the investigator's opinion at least one or more of the criteria are not present, then is can be assessed as “probably” related. Possibly Related—The event should be assessed following the same criteria for
“Definitely Related”. If in the investigator's opinion at least one or more of the criteria are not present, then it can be assessed as “possibly” related.
Probably Not Related—The event occurred while the participant was receiving study drug/intervention or undergoing study procedures but can reasonably be explained by the known characteristics of the participant's clinical state or other therapies.
Definitely Not Related—The event is definitely produced by the participant's clinical state or by other therapies administered to the participant.
Uncertain Relationship—The event does not meet any of the criteria previously outlined.

The investigator responsible at each local site will be responsible for determining whether an AE is expected or unexpected. An AE will be considered unexpected if the nature, severity, or frequency of the event is not consistent with the risk information previously described for the study agent.

There are a plethora of adverse events expected that will be related to COVID-19 infection and not the use of the drug, including death. Some examples of expected COVID-19 related AE include (but not limited to) death, intubation, need for pressors, mechanical circulatory support, resuscitated cardiac arrest, acute kidney injury, infection (non COVID-19), >5×LAN, disseminated intravascular coagulation, and symptomatic venous thromboembolism.

A total sample size of approximately 400 participants (Phase 2 combined with Phase 3, or Phase 3 alone) randomized 1:1 using permuted block randomization (˜200 bardoxolone methyl; ˜200 placebo) is expected to provide approximately 80% power at a two-sided 0.05 significance level using a generalized linear model analysis to detect a risk ratio of 1.28, corresponding to a 28% increase in probability of recovery, assuming the following:
Follow-up for events for 28 days after randomization (through Day 29)
2% of patients in each group drop-out prior to recovery
Increase in the probability of recovery by 28% corresponds to recovery for 50% patients randomized to placebo relative to 64% of patients randomized to bardoxolone methyl.

Because of the uncertainty around proportion recovered for COVID-19 patients, the protocol allows for the recalculation of the sample size during the trial. At a designated time during the trial (˜70% of projected sample size has accrued) interim analysis will be performed for efficacy, safety, futility and possible sample size recalculation. A simulation study will be used to estimate power based on the primary analysis method accounting for the competing event of death. The sample size will be adjusted, if necessary, to maintain desired power. The details of the sample size recalculation will be specified in the statistical analysis plan. Because these analyses will be based on pooled, blinded data, the recalculation will not affect the Type 1 error rate, nor will the data integrity of the study be affected.

The analysis will be based on intention to treat using a generalized linear model analysis to compare the probability of recovery within 29 days. The phase 2 analysis for safety will not assess the primary endpoint and will not impact the overall type I error rate of the trial. If the DSMB concludes the trial should continue to the Phase 3 part, then the phase 2 patients will be included in the phase 3 portion of the study (unless Phase 2 is unblinded).

eGFR measurement. The eGFR value will be calculated using the Chronic Kidney Disease Epidemiology Collaboration (CKD-EPI) equation:

eGFR (mL/min/1.73 m²)=141×min (S_{cr}/κ,1)^{α}×max(S_{cr}/κ,1)^{-1.209}×0.993^{Age}×1.018 [if female]×1.159 [if black]

where the patient's age on the date of consent is used, S_{cr} is serum creatinine (mg/dL), κ is 0.7 for females or 0.9 for males, and α is −0.329 for females or −0.411 for males. Min indicates the minimum of S_{cr}/κ or 1 and max indicates the maximum of S_{cr}/κ or 1.

PaO₂/FiO₂ Ratio. PaO2/FiO2 ratio will be calculated by using the PaO₂ from the arterial blood gas and then dividing by the estimated FiO₂ for that oxygen delivery method (0.21 for room air, 0.21+(oxygen flow rate*0.03) for nasal cannula, 0.80 for non-rebreather mask or the recorded FiO2 for non-invasive or invasive ventilation) (Brown et al., 2016).

Fluid Overload. Similar to endothelin receptor antagonists (ERAs) in certain patient populations, including bosentan in advanced congestive heart failure and avosentan in advanced CKD, bardoxolone methyl treatment was found to be associated with an increased risk for fluid overload and heart failure hospitalizations in the BEACON trial, which enrolled patients with Stage 4 CKD (eGFR 15 to 29 mL/min/1.73 m²) and type 2 diabetes (Chin, 2014). The overall increased risk for fluid overload and heart failure events with bardoxolone methyl appeared to be limited to the first three to four weeks after initiation of treatment. Patients with fluid overload events who were treated with intravenous diuretics generally resolved their symptoms. Elevated BNP and prior hospitalization for heart failure were identified as risk factors that contributed to increased risk for these events. For patients without these baseline characteristics, the risk for heart failure events among bardoxolone methyl- and placebo-treated patients was similar (2%) (Chin, 2014). The increased risk for these events from bardoxolone methyl treatment had also not been observed in six previous CKD studies, which were conducted mostly in patients with Stage 3b CKD (eGFR of 30 to 44 mL/min/1.73 m²), patients with hepatic dysfunction, cancer patients, or healthy volunteers.

Subsequent studies, enrolling over 1500 patients, have employed risk mitigation procedures to reduce the potential for bardoxolone methyl-induced fluid overload. These procedures excluded patients with the identified risk factors and ensured close monitoring for fluid retention within the first month of treatment. Since these exclusions have been applied, no increased risk for acute fluid overload AEs with bardoxolone methyl has been observed.

With regard to managing fluid status, specific risk mitigation procedures will be employed to reduce the potential for bardoxolone methyl-induced fluid overload. These include exclusion of patients with any severe renal disease, defined as an eGFR value of <15 mL/min/1.73 m² or those requiring dialysis. To exclude patients with significant cardiac dysfunction, the study will exclude patients with a known left ventricular ejection fraction (LVEF)<40% or prior hospitalization for heart failure.

Transaminase and Gamma-glutamyl Transpeptidase (GGT) Elevations. In clinical studies of bardoxolone methyl, almost all patients had increases of transaminase enzymes above baseline upon initiation of treatment, which followed a consistent pattern. These increases were not associated with elevations in bilirubin or other signs of liver toxicity. In BEACON, fewer hepatobiliary SAEs were observed in the bardoxolone methyl arm than in the placebo arm. The elevations begin immediately after initiation of treatment or an increase in dose; they peak approximately two to four weeks later. In most patients, transaminase elevations were mild, but approximately 4% to 11% of patients experienced an elevation greater than 3× the ULN. The elevations resolved to levels less than the ULN in most all patients with elevations, within two weeks after peak values while patients continued taking study drug. Patients who experienced elevations to greater than 3× the ULN sometimes required additional time to resolve. While some patients have had elevations to above 3× the ULN, persistent elevations to above 3× the ULN have not been observed, and the elevations did not recur once resolved, unless caused by other factors.

Bardoxolone methyl regulates GGT, a known Nrf2 target gene. In clinical studies, low level GGT elevations during treatment were common, mild, and typically lasted longer than ALT/AST elevations. Bilirubin levels in patients experiencing transaminase or GGT elevations due to treatment with bardoxolone methyl either remained at baseline levels or decreased. The ALT, AST, and GGT elevations were generally self-limiting in patients who continued treatment with study drug.

With regard to managing elevated transaminase levels (ALT and/or AST) in this trial, nearly all instances of elevated transaminases due to bardoxolone methyl treatment are expected to be asymptomatic. If ALT or AST levels are found to be more than times greater than ULN, then transaminase levels (as well as total bilirubin (TBL), GGT, alkaline phosphatase (ALP), and International Normalized Ratio (INR)) will be checked within 48 to 72 hours. Repeat testing will be performed every 72 to 96 hours until transaminase levels are below 5× the upper limit of normal (ULN) for at least one week or until the patient withdraws consent.

Muscle Spasms. Muscle spasm was the most frequently reported adverse event in clinical trials of bardoxolone methyl in patients with CKD who also had type 2 diabetes. The muscle spasms most often manifested in the first two months of treatment and resolved spontaneously or with empirical treatment. They occurred mostly at night, in the lower extremities, and were generally mild to moderate in severity. Muscle spasms may result from improved insulin sensitivity and glucose uptake in skeletal muscle cells. Increases in glucose uptake, as assessed by the hyperinsulinemic-euglycemic clamp procedure, were observed in response to bardoxolone methyl in a defined subset of patients enrolled in a Phase 2a study. Clinical signs and laboratory findings associated with the reports of muscle spasms have not been consistent with muscle toxicity. Bardoxolone methyl subjects showed no increase in prominent laboratory findings associated with muscle toxicity, such as increased levels of serum markers, including creatinine, creatine kinase, lactate dehydrogenase (LDH), BUN, uric acid, phosphorus, and potassium.

With regard to managing muscle spasms in this trial, basic symptomatic relief is the first step in managing muscle spasm, including walking, adequate hydration, wearing socks, and stretching before bedtime. Assessment of levels of electrolytes such as magnesium, calcium, and potassium may indicate the need for replacement. If vitamin D levels are low, supplementation may be warranted. Muscle relaxants may also help relieve symptoms.

TABLE 24
Schedule of Assessments

| | | Day | Day | Day | Day | Day | Day | Day | Day | End of | Day 60 Safety |
|---|---|---|---|---|---|---|---|---|---|---|---|
| Assessment | Screening | 1ª | 3 | 5 | 8 | 11 | 15 | 22 | 29^{k} | Treatment ^{j} | Follow-up^{b} |
| Informed | X | | | | | | | | | | |
| consent | | | | | | | | | | | |
| Inclusion/ | X | | | | | | | | | | |
| exclusion | | | | | | | | | | | |
| Demographics | X | | | | | | | | | | |
| and baseline | | | | | | | | | | | |
| disease | | | | | | | | | | | |
| characteristics | | | | | | | | | | | |
| Chest imaging | X^{h} | | | | | | | | | | |
| Medical | X | | | | | | | | | | |
| history | | | | | | | | | | | |
| Pregnancy test | X | | | | | | | | | | |
| for WOCBP^{e} | | | | | | | | | | | |
| Concomitant | X | X^{b,c} | X | X | X | X | X | X | | X | |
| medications | | | | | | | | | | | |
| Vital sign | X | X^{d,e} | X | X | X | X | X | X | X | X | X |
| measurements | | | | | | | | | | | |
| Study drug | | -------------------------------- X -------------------------------- | | | | | | | | | |
| administration | | | | | | | | | | | |
| Adverse event | | X ^{f} | X | X | X | X | | X | X | X | X |
| collection | | | | | | | | | | | |
| Recovery | | | | | | | | | | X | |
| status | | | | | | | | | | | |
| assessment | | | | | | | | | | | |
| Clinical status | X | X^{f,g} | X | X | X | X | X | X | X | X | X |
| assessment | | | | | | | | | | | |
| Comprehensive | X | X ^{d} | | X | X | | | X | X | X | X |
| metabolic | | | | | | | | | | | |
| panel | | | | | | | | | | | |
| Lipid panel | | X ^{d} | | X | X | | | X | | X | |
| Cytokines | | X ^{d} | | X | X | | | X | | X | |
| Ferritin | | X ^{d} | | X | X | | | X | | X | |
| Lactate | | X ^{d} | | X | X | | | X | | X | |
| dehydrogenase | | | | | | | | | | | |
| (LDH) | | | | | | | | | | | |
| C-reactive | | X ^{d} | | X | X | | | X | | X | |
| protein (CRP) | | | | | | | | | | | |
| Cardiac | | X ^{d} | | X | X | | | X | | X | |
| markers | | | | | | | | | | | |
| (Troponin I, | | | | | | | | | | | |
| CK) | | | | | | | | | | | |
| BNP | | X^{h,i} | X | X | X | X | X | X | X | X | X |
| Complete | | X^{i,k} | X | X | | | X | X | X | X | X |
| blood count | | | | | | | | | | | |
| (CBC) | | | | | | | | | | | |
| CBC | | X^{l,m} | X | X | X | X | X | X | X | X | X |
| differential | | | | | | | | | | | |
| PT & INR | | X^{n,o} | X | X | X | X | X | X | | X | |
| Fibrinogen | | X^{p,q} | X | X | X | X | X | X | | X | |
| D-dimer | | X^{r,s} | X | X | X | X | X | X | X | X | X |
| PaO₂/FiO₂ | | X ^{d} | X | X | X | X | X | X | | X | |
| SARS COV-2 | Xⁱ | | | | | | X^{g} | | | X ^{g} | |
| PCR Test | | | | | | | | | | | |

^{a}Day 1 is the day of administration of the first dose.
^{b}Patients who discontinue from study drug prior to recovery or prior to Day 29 will have a safety assessment 60 days after randomization. This will be in-person visit when possible or telephone follow-up.
^{c}Screening assessments conducted on the same day as randomization (Day 1) do not have to be repeated.
^{d}Performed pre-dose on Day 1.
^{e}A serum pregnancy test will be performed at the Screening visit for WOCBP or at any point in time if a pregnancy is suspected. Additional pregnancy assessments will be performed more frequently if required by local law or requested by local regulatory authorities or IRBs/ECs.
^{f} AE assessments on Day 1 should be performed following study drug administration. Only AEs that are serious and have a reasonable possibility of having been related to a study drug will be collected.
^{g}To be performed on Day 15 or End of Treatment, whichever is earlier.
^{h}Results available from test performed as per standard of care can be reviewed for eligibility. If not available or not done, it is not required to be performed.
ⁱAt screening, this test does not need to be performed. Only need to confirm a positive test is available to meet the study's inclusion criteria.
^{j} End of Treatment visit can occur on Day 29 or earlier. If the End of Treatment visit is on Day 29, follow schedule of assessments for End of Treatment visit.
^{k}All patients, regardless of treatment adherence and recovery status prior to Day 29, will have a follow-up visit at Day 29. This will be in-person visit when possible or telephone follow-up.

### Example 2—Results of Phase II Trial

The Phase 2 portion of the trial included 40 patients and was primarily designed to provide an initial assessment of the safety of bardoxolone methyl in COVID-19 patients when compared with matching placebo. The patient were all hospitalized with lab-confirmed COVID-19, and met at least one of the following criteria: (a) radiographic infiltrates by imaging; (b) at rest, blood oxygen saturation ≤94%; (c) required supplemental oxygen; (d) required non-invasive ventilation; and (e) required invasive mechanical ventilation for up to two days. Patients that were intubated and on invasive mechanical ventilation for three or more days were excluded. The demographics and baseline characteristics were similar between the placebo and bardoxolone methyl treatment groups (Table 25). There were no meaningful differences in COVID-19 related or current standard of care therapies between treatment groups (Table 26).

TABLE 25
Patient Demographics

| | Placebo | | Bard | | |
|---|---|---|---|---|---|
| | (n = 19) | | (n = 21) | | P-value |
| Age (years), mean ± SD | 61.3 ± 12.9 | | 57.3 ± 16.3 | | 0.40 |
| <30, n (%) | — | | — | | |
| 30 to <50, n (%) | 4 | (21.1) | 7 | (33.3) | |
| 50 to <70, n (%) | 10 | (52.6) | 10 | (47.6) | |
| ≥70, n (%) | 5 | (25.3) | 4 | (19.0) | |
| Male, n (%) | 13 | (68.4) | 12 | (57.1) | 0.53 |
| Hispanic or Latino, n (%) | 6 | (31.6) | 5 | (23.8) | 0.73 |
| Race, n (%) | | | | | 0.67 |
| White | 11 | (57.9) | 16 | (76.2) | |
| Black or African American | 3 | (15.8) | 1 | (4.8) | |
| Asian | 1 | (5.3) | 1 | (4.8) | |
| American Indian or Alaska | 1 | (5.3) | | | |
| Native | | | | | |
| Other | 3 | (15.8) | 3 | (14.3) | |
| Invasive Mechanical | — | | — | | |
| Ventilation Use | | | | | |
| WHO Score, Mean ± SD | 5.1 ± 0.74 | | 4.9 ± 0.57 | | 0.26 |
| Median (Q1, Q3) | 5.0 | (5.0, 6.0) | 5.0 | (5.0, 5.0) | |

TABLE 26
COVID-19 Related Medications of Interest

| | Placebo | Bard |
|---|---|---|
| | (n = 19) | (n = 21) |
| Number of patients who used any medication of | 18 (94.7) | 19 (90.5) |
| interest | | |
| Remdesivir | 14 (73.7) | 16 (76.2) |
| Steroids | 18 (94.7) | 19 (90.5) |
| Tocilizumab | 1 (5.3) | 1 (4.8) |
| Convalescent Plasma | 3 (15.8) | 4 (19) |

A patient is counted in each cell if they were recorded in either the baseline medications form or the concomitant medications log as having taken the medication at any point.

Forty patients were enrolled in the study and randomized 1:1 into either the placebo or bardoxolone methyl treatment groups. Two patients randomized to placebo did not receive study drug and were excluded from the mITT and Safety Populations. Patient disposition is shown in Table 27.

TABLE 27
Disposition

| | Placebo | Bard |
|---|---|---|
| ITT Population | 19 | 21 |
| mITT Population | 17 | 21 |
| Safety Population | 17 | 21 |
| Discontinued treatment | 4 (21.1) | 1 (4.8) |
| Adverse event | 1 (5.3) | — |
| Physician decision | 1 (5.3) | — |
| Withdrawal by subject | 2 (10.5) | 1 (4.8) |

Efficacy Endpoints: Fewer patients treated with bardoxolone methyl died, with an odds ratio of 0.19 for all-cause mortality (Table 28). Likewise, more bardoxolone methyl-treated patients were recovered at Day 29 with an odds ratio of 0.17 (Table 29). Median days from randomization to hospital discharge was reduced from 8 days in placebo patients to 5 days in bardoxolone methyl-treated patients (Table 30). Mean and median WHO scores were lower in the bardoxolone methyl-treated group at Day 29 (Table 31). Bardoxolone methyl treatment increased eGFR relative to placebo (Table 32).

TABLE 28
All-Cause Mortality and Deterioration

| | Placebo | Bard | |
|---|---|---|---|
| | (n = 19) | (n = 21) | |
| All-cause mortality | | | |
| Yes | 4 (21.1) | 1 | (4.8) |
| No | 15 (78.9) | 20 | (95.2) |
| Odds ratio (95% CI) | | 0.19 | (0.004, 2.2) |
| Day 29 Deterioration | | | |
| Yes | 4 (21.1) | 1 | (4.8) |
| No | 15 (78.9) | 20 | (95.2) |
| Odds ratio (95% CI) | | 0.19 | (0.004, 2.2) |

TABLE 29
Recovery and Modified Recovery (ITT Analysis1)

| | Placebo | Bard | |
|---|---|---|---|
| | (n = 19) | (n = 21) | |
| Recovery² | | | |
| Yes | 15 (78.9) | 1 | (4.8) |
| No | 15 (78.9) | 20 | (95.2) |
| Odds ratio (p-value) | | 0.19 | (0.004, 2.2) |
| Modified Recovery³ | | | |
| Yes | 4 (21.1) | 1 | (4.8) |
| No | 15 (78.9) | 20 | (95.2) |
| Odds ratio (p-value) | | 0.19 | (0.004, 2.2) |

1Includes all patients at Day 29, irrespective of status at baseline or prior to Day 29
²Recovery = Alive, free of respiratory failure [e.g., need for non-invasive or invasive mechanical ventilation, high flow oxygen, or ECMO], and free of renal replacement therapy [RRT]) at Day 29 (WHO ordinal scale score ≤5 and no RRT)
³Modified Recovery = Alive, free of any supplemental oxygen therapy, and free of renal replacement therapy [RRT]) at Day 29 (WHO ordinal scale score ≤4 and no RRT)

TABLE 30
Study Day of Hospital Discharge

| | Placebo | Bard |
|---|---|---|
| Days Hospitalized | (n = 19) | (n = 21) |
| Mean (SD) | 7.3 (3.54) | 6.3 (6.51) |
| Median | 8.0 | 5.0 |
| IQR | 4.0, 10.0 | 2.0, 7.0 |
| Min, Max | 2, 13 | 2, 29 |

TABLE 31
WHO Score at Day 29

| | Placebo | Bard |
|---|---|---|
| WHO Score | (n = 19) | (n = 21) |
| Mean (SD) | 3.2 (3.84) | 1.9 (2.23) |
| Median | 2.0 | 1.0 |
| Q1, Q3 | 0.0, 5.0 | 1.0, 2.0 |
| Min, Max | 0, 10 | 0, 10 |

TABLE 32
eGFR Change from Baseline

| | | Placebo | Bard | |
|---|---|---|---|---|
| ITT Population | Statistic | (n = 19) | (n = 21) | |
| Baseline (Result) | n | 18 | 20 | |
| | Mean (SD) | 87.5 (17.5) | 89.0 | (22.0) |
| | Median | 92.2 | 87.0 | |
| Day 1 (CFB) | n | 3 | 2 | |
| | Mean (SD) | 1.3 (3.8) | −3.0 | (9.0) |
| | Median | 0.0 | −3.0 | |
| Day 5 (CFB) | n | 12 | 11 | |
| | Mean (SD) | 3.5 (9.7) | 8.0 | (16.8) |
| | Median | 3.8 | 7.6 | |
| Day 8 (CFB) | n | 8 | 4 | |
| | Mean (SD) | 0.3 (15.9) | 5.0 | (3.3) |
| | Median | 0.6 | 4.8 | |
| Day 22 (CFB) | n | — | 1 | |
| | Mean (SD) | | 0.4 | (0) |
| | Median | | 0.4 | |
| End of Treatment (CFB) | n | 13 | 13 | |
| | Mean (SD) | −1.0 (15.1) | 2.9 | (7.2) |
| | Median | 0.5 | 2.3 | |
| End of Treatment-LOCF | n | 18 | 20 | |
| (CFB) | Mean (SD) | −0.9 (12.9) | 4.7 | (12.7) |

| | | |
|---|---|---|
| Median | −0.2 | 1.7 |

Safety Endpoints: A lower percentage of bardoxolone methyl (24%) versus placebo (35%) patients experienced an AE (Table 33). Likewise, a lower percentage of bardoxolone methyl (19%) versus placebo (35%) patients experienced an SAE (Table 33). No cardiovascular SAEs occurred in bardoxolone methyl-treated patients (Table 34). No SAEs were related to study drug. No other safety findings or adverse lab trends were observed.

TABLE 33
Adverse Event Overview (Safety Population)

| | Placebo | Bard |
|---|---|---|
| Adverse Event Overview | (n = 17) | (n = 21) |
| Number of Patients with AE | 6 (35.3) | 5 (23.8) |
| Number of Patients with related AE | — | — |
| Number of Patients with severe AE | 3 (17.6) | 3 (14.3) |
| Number of Patients with SAE | 6 (35.3) | 4 (19.0) |
| Number of Patients with related-SAE | — | — |
| Discontinuations due to AEs | 1 | — |

TABLE 34
Serious Adverse Events (Safety Population)

| Serious Adverse Events | Placebo | Bard |
|---|---|---|
| Preferred term (System organ class) | (n = 17) | (n = 21) |
| Patients reporting at least one SAE | 6 (35.3) | 4 (19.0) |
| Pneumonia viral (Infections and infestations | 2 (11.8) | 1 (4.8) |
| Cardiac failure acute (Cardiac disorders) | 1 (5.9) | — |
| Ventricular tachycardia (Cardiac disorders) | 1 (5.9) | — |
| Death (General disorders and administration site | 1 (5.9) | — |
| conditions) | | |
| Abdominal wall wound (Injury, poisoning and | — | 1 (4.8) |
| procedural complications) | | |
| Splenic rupture (Injury, poisoning and procedural | — | 1 (4.8) |
| complications) | | |
| Presyncope (Nervous system disorders) | 1 (5.9) | — |
| Nephrolithiasis (Renal and urinary disorders) | — | 1 (4.8) |
| Hypoxia (Respiratory, thoracic and mediastinal | 1 (5.9) | — |
| disorders) | | |
| Pulmonary embolism (Respiratory, thoracic and | 1 (5.9) | — |
| mediastinal disorders) | | |
| Mechanical ventilation (Surgical and medical | 1 (5.9) | 1 (4.8) |
| procedures) | | |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,480,792
U.S. Pat. No. 5,525,524
U.S. Pat. No. 5,631,170
U.S. Pat. No. 5,679,526
U.S. Pat. No. 5,824,799
U.S. Pat. No. 5,851,776
U.S. Pat. No. 5,885,527
U.S. Pat. No. 5,922,615
U.S. Pat. No. 5,939,272
U.S. Pat. No. 5,947,124
U.S. Pat. No. 5,955,377
U.S. Pat. No. 5,985,579
U.S. Pat. No. 6,019,944
U.S. Pat. No. 6,025,395
U.S. Pat. No. 6,113,855
U.S. Pat. No. 6,143,576
U.S. Pat. Pub. 2003/0232786
U.S. Pat. Pub. 2008/0261985
U.S. Pat. Pub. 2009/0048204
U.S. Pat. Pub. 2009/0326063
U.S. Pat. Pub. 2010/0041904
U.S. Pat. Pub. 2010/0048887
U.S. Pat. Pub. 2010/0048892
U.S. Pat. Pub. 2010/0048911
U.S. Pat. Pub. 2010/0056777
U.S. Pat. Pub. 2011/0201130
PCT Pub. WO 2009/023232
PCT Pub. WO 2009/048204
PCT Pub. WO 2010/093944
PCT Pub. WO 2019/014412
CN 102875634
CN 102887936
Ahmad et al., “Triterpenoid CDDO-Me blocks the NF-κB pathway by direct inhibition of IKKβ on Cys-179,” J. Biol. Chem., 281:35764-35769, 2006.
Ahmad et al., “Triterpenoid CDDO-Methyl Ester Inhibits the Janus-Activated Kinase-1 (JAK1)→Signal Transducer and Activator of Transcription-3 (STAT3) Pathway by Direct Inhibition of JAK1 and STAT3,” Cancer Res., 68(8):2920-2926, 2008.
Ahmadppor & Rostaing, “Why the immune system fails to mount an adaptive immune response to a COVID-19 infection.” Transpl. Int., Apr. 1, 2020, doi: 10.111/tri.13611.
Aleksunes et al., “Transcriptional regulation of renal cytoprotective genes by Nrf2 and its potential use as a therapeutic target to mitigate cisplatin-induced nephrotoxicity,” J. Pharmacol. Exp. Ther., 335(1):2-12, 2010.
Aminzadeh et al., “The synthetic triterpenoid RTA dh404 (CDDO-dhTFEA) restores endothelial function impaired by reduced Nrf2 activity in chronic kidney disease,” Redox Biol., 1:527-531, 2013.
Aminzadeh et al., “The synthetic triterpenoid RTA dh404 (CDDO-dhTFEA) restores Nrf2 activity and attenuates oxidative stress, inflammation, and fibrosis in rats with chronic kidney disease,” Xenobiotica, 44(6):570-578, 2014.
Anderson, Practical Process Research & Development—A Guide for Organic Chemists, 2^{nd} ed., Academic Press, New York, 2012.
Auletta et al., “The synthetic triterpenoid, CDDO-Me, modulates the proinflammatory response to in vivo lipopolysaccharide challenge,” J. Interferon Cytokine Res., 30:497-508, 2010.
Brown et al., “Nonlinear imputation of Pao2/Fio2 from Spo2/Fio2 among patients with acute respiratory distress syndrome,” Chest, 150(2):307-313, 2016.
Camer et al., “Bardoxolone methyl prevents the development and progression of cardiac and renal pathophysiologies in mice fed a high-fat diet,” Chem. Biol. Interact., 243:10-18, 2016.
Chandra et al., “Mesenchymal stem cells are attracted to latent HIV-1-infected cells and enable virus reactivation via a non-canonical PI3K-NFκB signaling pathway,” Sci. Rep., 8:14702, 2018.
Chapman et al., “Cyclic mechanical strain increases reactive oxygen species production in pulmonary epithelial cells,” Am. J. Physiol. Lung Cell. Mol. Physiol., 289:L834-841, 2005.
Chen et al., “The protective effect of CDDO-Me on lipopolysaccharide-induced acute lung injury in mice,” Int. Immunopharmacol., 25:55-64, 2015.
Chen et al., “Epidemiological and clinical characteristics of 99 cases of 2019 novel coronavirus pneumonia in Wuhan, China: a descriptive study,” The Lancet, 395:507-513, 2020.
Chen et al., “Clinical and immunological features of severe and moderate coronavirus disease 2019,” J. Clin. Invest., Mar. 27, 2020, doi: 10.1172/JCI137244.
Chen et al., “SARS coronavirus papain-like protease inhibits the type I interferon signaling pathway through interaction with the STING-TRAF3-TBK1 complex,” Protein Cell, 5:369-381, 2014.
Chertow et al., “Effects of Bardoxolone Methyl on Body Weight, Waist Circumference and Glycemic Control in Obese Patients with Type 2 Diabetes Mellitus and Stage 4 Chronic Kidney Disease,” J. Diabetes Complications, 32(12):1113-1117, 2018.
Chin et al., “Bardoxolone Methyl Improves Kidney Function in Patients with Chronic Kidney Disease Stage 4 and Type 2 Diabetes: Post-Hoc Analyses from Bardoxolone Methyl Evaluation in Patients with Chronic Kidney Disease and Type 2 Diabetes Study,” Am. J. Nephrol., 47:40-47, 2018.
Chin et al., “Bardoxolone methyl analogs RTA 405 and dh404 are well tolerated and exhibit efficacy in rodent models of Type 2 diabetes and obesity,” Am. J. Physiol. Renal Physiol., 304:F1438-F46, 2013.
Chin et al., “Mechanisms contributing to adverse cardiovascular events in patients with type 2 diabetes mellitus and stage 4 chronic kidney disease treated with bardoxolone methyl,” Am. J. Nephrol., 39:499-508, 2014.
de Zeeuw et al., “Bardoxolone methyl in type 2 diabetes and stage 4 chronic kidney disease. N. Engl. J. Med., 369:2492-2503, 2013.
Dhaun et al., “Urinary endothelin-1 in chronic kidney disease and as a marker of disease activity in lupus nephritis,” American Journal of Physiology—Renal Physiology, 296:F1477-F1483, 2009.
Diao et al., “Reduction and Functional Exhaustion of T Cells in Patients with Coronavirus Disease 2019 (COVID-19),” Feb. 20, 2020, medRxiv, doi:10.1101/2020.02.18.20024364.
Ding et al., “The synthetic triterpenoid, RTA 405, increases the glomerular filtration rate and reduces angiotensin II-induced contraction of glomerular mesangial cells,” Kidney Int., 83:845-854, 2013.
Dinkova-Kostova et al., “The spatiotemporal regulation of the Keap1-Nrf2 pathway and its importance in cellular bioenergetics,” Biochem. Soc. Trans., 43:602-610, 2015.
Dinkova-Kostova et al., “Extremely Potent Triterpenoid Inducers of the Phase 2 Response: Correlations of Protection Against Oxidant and Inflammatory Stress,” Proc. Natl. Acad. Sci., 102:4584-4589, 2005.
Espinoza et al., “Modulation of Antiviral Immunity by Heme Oxygenase-1,” Am. J. Pathol., 187:487-493, 2017.
Fanelli et al., “Acute kidney injury in SARS-CoV-2 infected patients,” Crit. Care, 24:155, 2020.
Ferguson et al., “Bardoxolone Methyl (BARD) Improves Markers of Endothelial Function in Cultured Cells,” Poster American Society of Nephrology (ASN) 2010.
Fung et al., “A tug-of-war between severe acute respiratory syndrome coronavirus 2 and host antiviral defence: lessons from other pathogenic viruses,” Emerg. Microbes Infect., 9:558-570, 2020.
Guan et al., “Clinical Characteristics of Coronavirus Disease 2019 in China,” N. Engl. J. Med., 382:1708-1720, 2020.
Handbook of Pharmaceutical Salts: Properties, and Use, Stahl and Wermuth Eds.), Verlag Helvetica Chimica Acta, 2002.
Hisamichi et al., “Role of bardoxolone methyl, a nuclear factor erythroid 2-related factor 2 activator, in aldosterone- and salt-induced renal injury,” Hypertens. Res., 41(1):8-17, 2018.
Honda et al., “New Enone Derivativas of Oleanolic Acid and Ursolic Acid as Inhibitors of Nitric Oxide Production in Mouse Macrophages,” 1997.
Honda et al., “Design and Synthesis of 2-cyano-3,12-dioxoolean-1,9-dien-28-oic acid, a Novel and Highly Active Inhibitor of Nitric Oxide Production in Mouse Macrophages,” Bioorg. Med. Chem. Lett., 8(19):2711-2714, 1998.
Honda et al., “Novel Synthetic Oleanane Triterpenoids: A Series of Highly Active Inhibitors of Nitric Oxide Production in Mouse Macrophages,” Bioorg. Med. Chem. Lett., 9(24):3429-3434, 1999.
Honda et al., “Novel Synthetic Oleanane and Ursane Triterpenoids with Various Enone Functionalities in Ring A as Inhibitors of Nitric Oxide Production in Mouse Macropahges,” J. Med. Chem., 43:1866-1877, 2000a.
Honda et al., “Synthetic Oleanane and Ursane Triterpenoids with Modified Rings A and C: A Series of Highly Active Inhibitors of Nitric Oxide Production in Mouse Macrophages,” J. Med. Chem., 43:4233-4246, 2000b.
Honda et al., “A novel dicyanotriterpenoid, 2-cyano-3,12-dioxooleana-1,9(11)-dien-28-onitrile, active at picomolar concentrations for Inhibition of Nitric Oxide Production,” Bioorg. Med. Chem. Lett., 12:1027-1030, 2002.
Hong et al., “A phase I first-in-human trial of bardoxolone methyl in patients with advanced solid tumors and lymphomas,” Clin. Cancer Res., 18:3396-3406, 2012.
Huang et al., “Clinical features of patients infected with 2019 novel coronavirus in Wuhan, China,” Lancet, 395:497-506, 2020.
Huang et al., “Inhibition of Skin Tumorigenesis by Rosemary and its Constituents Carnosol and Ursolic Acid,” Cancer Res., 54:701-708, 1994.
Ikeda et al., “The Novel Triterpenoid CDDO and its Derivatives Induce Apoptosis by Disruption of Intracellular Redox Balance,” Cancer Res., 63:5551-5558, 2003.
Ikeda et al., “Induction of Redox Imbalance and Apoptosis in Multiple Myeloma Cells by the Novel Triterpenoid 2-cyano-3,12-dioxoolean-1,9-dien-28-oic acid,” Mol. Cancer Ther., 3:39-45, 2004.
Joint Specialty Committee on Renal Medicine of the Royal College of Physicians and the Renal Association, and the Royal College of General Practitioners. Chronic kidney disease in adults: UK guidelines for identification, management and referral. London: Royal College of Physicians, 2006.
Keleku-Lukwete et al., “Amelioration of inflammation and tissue damage in sickle cell model mice by Nrf2 activation,” Proc. Natl. Acad. Sci. USA, 112:12169-12174, 2015.
Kellner et al., “ROS Signaling in the Pathogenesis of Acute Lung Injury (ALI) and Acute Respiratory Distress Syndrome (ARDS),” Adv. Exp. Med. Biol., 967:105-137, 2017.
Kellner et al., In: Wang Y-X, ed. Pulmonary Vasculature Redox Signaling in Health and Disease. Cham: Springer International Publishing; pp. 105-137, 2017.
Kobayashi & Yamamoto, “Molecular Mechanism Activating the Nrf2-Keap 1 Pathway of Antioxidant Gene Regulation,” Antioxid. Redox. Signal., 7:385-394, 2005.
Kulkarni et al., “The triterpenoid CDDO-Me inhibits bleomycin-induced lung inflammation and fibrosis,” PLoS One, 8:e63798, 2013.
Lee et al., “KEAP1 E3 ligase-mediated downregulation of NF-kB signaling by targeting IKKβ,” Molecular Cell, 36:131-140, 2009.
Liby et al., “The Synthetic Triterpenoids, CDDO and CDDO-imidazolide, are Potent Inducers of Heme Oxygenase-1 and Nrf2/ARE signaling,” Cancer Res., 65:4789-4798, 2005.
Liu et al., “The Nrf2 triterpenoid activator, CDDO-imidazolide, protects kidneys from ischemia-reperfusion injury in mice,” Kidney Int., 85(1):134-141, 2014.
Liu et al., “2019-novel coronavirus (2019-nCoV) infections trigger an exaggerated cytokine response aggravating lung injury,” Feb. 12, 2020, chinaXiv, doi:10/12074/202002.00018/
Maines & Gibbs, “30 Some Years of Heme Oxygenase: From a ‘molecular wrecking ball’ to a ‘mesmerizing’ Trigger of Cellular Events,” Biochem. Biophys. Res. Commun., 338:568-577, 2005.
March's Advanced Organic Chemistry: Reactions, Mechanisms, and Structure, 2007.
Nagashima et al., “Nrf2 Suppresses Allergic Lung Inflammation by Attenuating the Type 2 Innate Lymphoid Cell Response,” J. Immunol., 202:1331-1339, 2019.
Nagasu et al., “Bardoxolone methyl analog attenuates proteinuria-induced tubular damage by modulating mitochondrial function,” FASEB J., 33(11):12253-12263, 2019.
Naicker et al., “The Novel Coronavirus 2019 epidemic and kidneys,” Kidney Int., 97:824-828, 2020.
Nangaku et al., “Effect of bardoxolone methyl on glomerular filtration rate in diabetic kidney disease patients (TSUBAKI study): a randomized clinical trial. Kidney International Reports. 2020; In press.
Nichols et al., “The triterpenoid CDDO limits inflammation in preclinical models of cystic fibrosis lung disease,” Am. J. Physiol. Lung Cell. Mol. Physiol., 297:L828-836, 2009.
Nio et al., “Bardoxolone methyl as a novel potent antiviral agent against hepatitis B and C viruses in human hepatocyte cell culture systems,” Antiviral Res., 169:104537, 2019.
Nishino et al., “Inhibition of the Tumor-Promoting Action of 12-O tetradecanoylphorbol-13-acetate by some Oleanane-type Triterpenoid Compounds,” Cancer Res., 48:5210-5215, 1988
Osburn & Kensler, “Nrf2 signaling: An adaptive response pathway for protection against environmental toxic insults,” Mutat. Res., 669:319, 2008.
Osburn et al., “Genetic or pharmacologic amplification of nrf2 signaling inhibits acute inflammatory liver injury in mice,” Toxicol. Sci., 104:218-227, 2008.
Papaiahgari et al., “Genetic and Pharmacologic Evidence Links Oxidative Stress to Ventilator-induced Lung Injury in Mice,” Am. J. Respir. Crit. Care Med., 176:1222-1235, 2007.
Patra et al., “RA-839, a selective agonist of Nrf2/ARE pathway, exerts potent anti-rotaviral efficacy in vitro,” Antiviral Res., 161:53-62, 2019.
Peake & Whiting, “Measurement of Serum Creatinine-Current Status and Future Goals,” Clin. Biochem. Rev., 27:173-184, 2006.
Pei et al., “Bardoxolone treatment alleviates lipopolysaccharide (LPS)-induced acute lung injury through suppressing inflammation and oxidative stress regulated by Nrf2 signaling,” Biochem. Biophys. Res. Commun., 516:270-277, 2019.
Pergola et al., “Bardoxolone methyl and kidney function in CKD with type 2 diabetes,” New Engl. J. Med., 365, 327-336, 2011.
Pergola et al., “Safety and efficacy of bardoxolone methyl in patients with rare chronic kidney diseases,” Poster European Renal Association-European Dialysis and Transplant Association (ERA-EDTA), 2019.
Place et al., “The Novel Synthetic Triterpenoid, CDDO-Imidazolide, Inhibits Inflammatory Response and Tumor Growth In Vivo,” Clin. Cancer Res., 9:2798-2806, 2003.
Potey et al., “Neutrophils in the initiation and resolution of acute pulmonary inflammation: understanding biological function and therapeutic potential,” J. Pathol., 247:672-685, 2019.
Qin et al., “Dysregulation of immune response in patients with COVID-19 in Wuhan, China,” Clin. Infect. Dis., Mar. 12, 2020, doi:10.1093/cid/ciaa248.
Rawlins et al., “Performance Characteristics of Four Automated Natriuretic Peptide Assays,” Am. J. Clin. Pathol., 123:439-445, 2005.
Reagan-Shaw et al., “Dose Translation From Animal to Human Studies Revisited,” FASEB J., 22(3):659-661, 2008.
Reddy et al., “The triterpenoid CDDO-imidazolide confers potent protection against hyperoxic acute lung injury in mice,” Am. J. Respir. Crit. Care Med., 180:867-874, 2009.
Repka et al., “Encyclopedia of Pharmaceutical Technology,” New York:Marcel Dekker, 2002.
Robles et al., “Synthetic Triterpenoid RTA dh404 (CDDO-dhTFEA) Ameliorates Acute Pancreatitis,” Pancreas, 45:720-729, 2016.
Rojas-Rivera et al., “Antioxidants in kidney diseases: The impact of bardoxolone methyl. Int J Nephrol 2012; 2012:1-11.
Rothan H A, Zhong Y, Sanborn M A, Teoh T C, Ruan J, Yusof R, et al. Small molecule grp94 inhibitors block dengue and Zika virus replication. Antiviral Res. 2019; 171:104590.
Sarzi-Puttini P, Giorgi V, Sirotti S, Marotto D, Ardizzone S, Rizzardini G, et al. COVID-19, cytokines and immunosuppression: what can we learn from severe acute respiratory syndrome? Clin Exp Rheumatol. 2020; 38(2):337-42.
Schneider et al., “Contrasting actions of endothelin ETA and ETB receptors in cardiovascular disease,” Annual Review of Pharmacology and Toxicology, 47:731-759, 2007.
Shao J, Huang J, Guo Y, Li L, Liu X, Chen X, et al. Up-regulation of nuclear factor E2-related factor 2 (Nrf2) represses the replication of SVCV. Fish & Shellfish Immunology. 2016; 58:474-82.
Shishodia et al., “A Synthetic Triterpenoid, CDDO-Me, Inhibits IκBα Kinase and Enhances Apoptosis Induced by TNF and Chemotherapeutic Agents Through Down-Regulation of Expression of Nuclear Factor KB-Regulated Gene Products in Human Leukemic Cells,” Clin. Cancer Res., 12(6):1828-1838, 2006.
Son et al., “Activation of Nrf2 Restores Klotho Expression and Attenuates Oxidative Stress and Inflammation in CKD,” J. Appl. Health Sci. Int., 2:22-34, 2015.
Suh et al., “Novel Triterpenoids Suppress Inducible Nitric Oxide Synthase (iNOS) and Inducible Cyclooxygenase (COX-2) in Mouse Macrophages,” Cancer Res., 58:717-723, 1998.
Suh et al., “A Novel Synthetic Oleanane Triterpenoid, 2-cyano-3,12-dioxoolean-1,9-dien-28-oic acid, with potent differentiating, antiproliferative, and anti-inflammatory activity,” Cancer Res., 59(2):336-341, 1999.
Suh et al., “Synthetic Triterpenoids Enhance Transforming Growth Factor β/Smad Signaling,” Cancer Res., 63:1371-1376, 2003.
Sun et al., “Coronavirus Papain-like Proteases Negatively Regulate Antiviral Innate Immune Response through Disruption of STING-Mediated Signaling,” PLoS One, 7:e30802, 2012.
Tan et al., “Derivative of bardoxolone methyl, dh404, in an inverse dose-dependent manner lessens diabetes-associated atherosclerosis and improves diabetic kidney disease,” Diabetes, 63(9):3091-3103, 2014.
Tanaka et al., “Coordinated induction of Nrf2 target genes protects against iron nitrilotriacetate (FeNTA)-induced nephrotoxicity,” Toxicol. Appl. Pharmacol., 231:364-373, 2008.
Thimmulappa et al., “Preclinical evaluation of targeting the Nrf2 pathway by triterpenoids (CDDO-Im and CDDO-Me) for protection from LPS-induced inflammatory response and reactive oxygen species in human peripheral blood mononuclear cells and neutrophils,” Antioxid. Redox Signal., 9:1963-1970, 2007.
Thimmulappa et al., “Nrf2-dependent protection from LPS induced inflammatory response and mortality by CDDO-Imidazolide,” Biochem. Biophys. Res. Commun., 351:883-889, 2006.
Vachiery & Davenport, “The endothelin system in pulmonary and renal vasculopathy: les liaisons dangereuses,” European Respiratory Review, 18:260-271, 2009.
Vasan et al., “Congestive heart failure in subjects with normal versus reduced left ventricular ejection fraction: Prevalence and mortality in a population-based cohort,” Journal of the American College of Cardiology, 33:1948-1955, 1999.
Vázquez et al., “Human immunodeficiency virus type 1-induced macrophage gene expression includes the p21 gene, a target for viral regulation,” J. Virol., 79:4479-4491, 2005.
Wang et al., “A Synthetic Triterpenoid, 2-cyano-3,12-dioxooleana-1,9-dien-28-oic acid (CDDO), is a Ligand for the Peroxisome Proliferator-Activated Receptor γ,” Mol. Endocrin., 14(10):1550-1556, 2000.
Wu et al., “Bardoxolone methyl (BARD) ameliorates ischemic AKI and increases expression of protective genes Nrf2, PPARgamma, and HO-1,” Am. J. Physiol. Renal Physiol., 300:F1180-F1192, 2011.
Wu et al., “Bardoxolone methyl (BARD) ameliorates aristolochic acid (AA)-induced acute kidney injury through Nrf2 pathway,” Toxicology, 318:22-31, 2014.
Wu et al., “Prevention of murine lupus nephritis by targeting multiple signaling axes and oxidative stress using a synthetic triterpenoid,” Arthritis Rheumatol., 66(11):3129-3139, 2014.
Wyler et al., “Single-cell RNA-sequencing of herpes simplex virus 1-infected cells connects NRF2 activation to an antiviral program,” Nat. Commun., 10:4878, 2019.
Yang et al., “Clinical course and outcomes of critically ill patients with SARS-CoV-2 pneumonia in Wuhan, China: a single-centered, retrospective, observational study,” Lancet Respir. Med., 8:475-481, 2020.
Yang et al., “Exuberant elevation of IP-10, MCP-3 and IL-1ra during SARS-CoV-2 infection is associated with disease severity and fatal outcome,” Mar. 6, 2020, medRxiv, doi:10.1101/2020.03.02.20029975.
Yates et al., “Pharmacodynamic Characterization of Chemopreventive Triterpenoids as Exceptionally Potent Inducers of Nrf2-regulated Genes,” Mol. Cancer. Ther., 6:154-162, 2007.
Zhang et al., “Nrf2 Activator RTA-408 Protects Against Ozone-Induced Acute Asthma Exacerbation by Suppressing ROS and γδT17 Cells,” Inflammation, 42:1843-1856, 2019.
Zhou et al., “Clinical course and risk factors for mortality of adult inpatients with COVID-19 in Wuhan, China: a retrospective cohort study,” Lancet, 395:1054-1062, 2020.
Zhao et al., “The role of nuclear factor-erythroid 2 related factor 2 (Nrf-2) in the protection against lung injury,” Am. J. Physiol Lung Cell. Mol. Physiol., 312:L155-162, 2017.

What is claimed is:

1. A method of treating a patient infected with SARS-CoV-2, comprising administering to the patient a therapeutically effective amount of a compound of the formula: 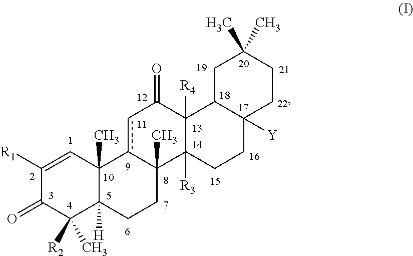
wherein:
R₁ is —CN, halo, —CF₃, or —C(O)Rₐ, wherein Rₐ is —OH, alkoxy_{(C1-4)}, —NH₂, alkylamino_{(C1-4)}, or —NH—S(O)₂-alkyl_{(C1-4)};
R₂ is hydrogen or methyl;
R₃ and R₄ are each independently hydrogen, hydroxy, methyl or as defined below when either of these groups is taken together with group R_{c}; and
Y is:
—H, —OH, —SH, —CN, —F, —CF₃, —NH₂ or —NCO;
alkyl_{(C≤8)}, cycloalkyl_{(C≤8)}, alkenyl_{(C≤8)}, alkynyl_{(C≤8)}, aryl_{(C≤12)}, aralkyl_{(C≤12)}, heteroaryl_{(C≤8)}, heterocycloalkyl_{(C≤12)}, alkoxy_{(C≤8)}, cycloalkoxy_{(C≤8)}, aryloxy_{(C≤12)}, acyloxy_{(C≤8)}, alkylamino_{(C≤8)}, cycloalkylamino_{(C≤8)}, dialkylamino_{(C≤8)}, arylamino_{(C≤8)}, aralkylamino_{(C≤8)}, alkylthio_{(C≤8)}, acylthio_{(C≤8)}, alkyl sulfonylamino_{(C≤8)}, cycloalkylsulfonylamino_{(C≤8)}, or substituted versions of any of these groups;
—alkanediyl_{(C≤8)}-R_{b}, —alkenediyl_{(C≤8)}-R_{b}, or a substituted version of any of these groups, wherein R_{b} is:
hydrogen, hydroxy, halo, amino or mercapto; or
heteroaryl_{(C≤8)}, alkoxy_{(C≤8)}, cycloalkoxy_{(C≤8)}, alkenyloxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, aralkylamino_{(C≤8)}, heteroarylamino_{(C≤8)}, alkylsulfonylamino_{(C≤8)}, cycloalkylsulfonylamino_{(C≤8)}, amido_{(C≤8)}, —OC(O)NH-alkyl_{(C≤8)}, or a substituted version of any of these groups;
—(CH₂)ₘC(O)R_{c}, wherein m is 0-6 and Re is:
hydrogen, hydroxy, halo, amino, —NHOH, or mercapto; or
alkyl_{(C≤8)}, cycloalkyl_{(C≤8)}, alkenyl_{(C≤8)}, alkynyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, cycloalkoxy_{(C≤8)}, alkenyloxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, alkylsulfonylamino_{(C≤8)}, cycloalkylsulfonylamino_{(C≤8)}, amido_{(C≤8)}, —NH-alkoxy_{(C≤8)}, —NH-heterocycloalkyl_{(C≤8)}, —NH-amido_{(C≤8)}, or a substituted version of any of these groups;
R_{c} and R₃, taken together, are —O— or —NR_{d}—, wherein R_{d} is hydrogen or alkyl_{(C≤4)}; or
R_{c} and R₄, taken together, are —O— or —NR_{d}—, wherein R_{d} is hydrogen or alkyl_{(C≤4)}; or
—NHC(O)Rₑ, wherein Rₑ is:
hydrogen, hydroxy, amino; or
alkyl_{(C≤8)}, cycloalkyl_{(C≤8)}, alkenyl_{(C≤8)}, alkynyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, cycloalkoxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, or a substituted version of any of these groups;
or a pharmaceutically acceptable salt thereof.

2. The method of claim 1, wherein the patient has been identified as not having a history of left-sided heart failure or wherein the patient does not show evidence of left ventricular dysfunction.

3. A method of treating a patient infected with a coronavirus, comprising administering to the patient a therapeutically effective amount of a compound of the formula: 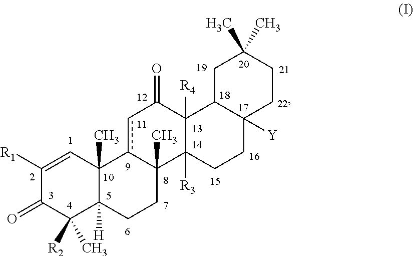
wherein:
R₁ is —CN, halo, —CF₃, or —C(O)Rₐ, wherein Rₐ is —OH, alkoxy_{(C1-4)}, —NH₂, alkylamino_{(C1-4)}, or —NH—S(O)₂-alkyl_{(C1-4)};
R₂ is hydrogen or methyl;
R₃ and R₄ are each independently hydrogen, hydroxy, methyl or as defined below when either of these groups is taken together with group R_{c}; and
Y is:
—H, —OH, —SH, —CN, —F, —CF₃, —NH₂ or —NCO;
alkyl_{(C≤8)}, cycloalkyl_{(C≤8)}, alkenyl_{(C≤8)}, alkynyl_{(C≤8)}, aryl_{(C≤12)}, aralkyl_{(C≤12)}, heteroaryl_{(C≤8)}, heterocycloalkyl_{(C≤12)}, alkoxy_{(C≤8)}, cycloalkoxy_{(C≤8)}, aryloxy_{(C≤12)}, acyloxy_{(C≤8)}, alkylamino_{(C≤8)}, cycloalkylamino_{(C≤8)}, dialkylamino_{(C≤8)}, arylamino_{(C≤8)}, aralkylamino_{(C≤8)}, alkylthio_{(C≤8)}, acylthio_{(C≤8)}, alkyl sulfonylamino_{(C≤8)}, cycloalkylsulfonylamino_{(C≤8)}, or substituted versions of any of these groups;
-alkanediyl_{(C≤8)}-R_{b}, -alkenediyl_{(C≤8)}-R_{b}, or a substituted version of any of these groups, wherein R_{b} is:
hydrogen, hydroxy, halo, amino or mercapto; or
heteroaryl_{(C≤8)}, alkoxy_{(C≤8)}, cycloalkoxy_{(C≤8)}, alkenyloxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, aralkylamino_{(C≤8)}, heteroarylamino_{(C≤8)}, alkylsulfonylamino_{(C≤8)}, cycloalkylsulfonylamino_{(C≤8)}, amido_{(C≤8)}, —OC(O)NH-alkyl_{(C≤8)}, or a substituted version of any of these groups;
—(CH₂)ₘC(O)R_{c}, wherein m is 0-6 and Rₑ is:
hydrogen, hydroxy, halo, amino, —NHOH, or mercapto; or
alkyl_{(C≤8)}, cycloalkyl_{(C≤8)}, alkenyl_{(C≤8)}, alkynyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, cycloalkoxy_{(C≤8)}, alkenyloxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, alkylsulfonylamino_{(C≤8)}, cycloalkylsulfonylamino_{(C≤8)}, amido_{(C≤8)}, —NH-alkoxy_{(C≤8)}, —NH— heterocycloalkyl_{(C≤8)}, —NH-amido_{(C≤8)}, or a substituted version of any of these groups;
R_{c} and R₃, taken together, are —O— or —NR_{d}—, wherein R_{d} is hydrogen or alkyl_{(C≤4)}; or
R_{c} and R₄, taken together, are —O— or —NR_{d}—, wherein R_{d} is hydrogen or alkyl_{(C≤4)}; or
—NHC(O)Rₑ, wherein Rₑ is:
hydrogen, hydroxy, amino; or
alkyl_{(C≤8)}, cycloalkyl_{(C≤8)}, alkenyl_{(C≤8)}, alkynyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, cycloalkoxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, or a substituted version of any of these groups;
or a pharmaceutically acceptable salt thereof,
wherein the compound is not

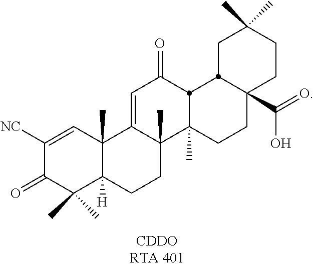

4. The method of claim 3, wherein the patient has been identified as not having a history of left-sided heart failure or wherein the patient does not show evidence of left ventricular dysfunction.

5. A method of treating a patient infected with a coronavirus, comprising administering to the patient a therapeutically effective amount of a compound of the formula: 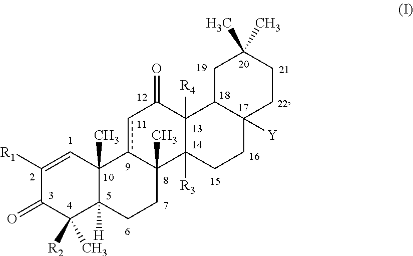
wherein:
R₁ is —CN, halo, —CF₃, or —C(O)Rₐ, wherein Rₐ is —OH, alkoxy_{(C1-4)}, —NH₂, alkylamino_{(C1-4)}, or —NH—S(O)₂-alkyl_{(C1-4)};
R₂ is hydrogen or methyl;
R₃ and R₄ are each independently hydrogen, hydroxy, methyl or as defined below when either of these groups is taken together with group R_{c}; and
Y is:
—H, —OH, —SH, —CN, —F, —CF₃, —NH₂ or —NCO;
alkyl_{(C≤8)}, cycloalkyl_{(C≤8)}, alkenyl_{(C≤8)}, alkynyl_{(C≤8)}, aryl_{(C≤12)}, aralkyl_{(C≤12)}, heteroaryl_{(C≤8)}, heterocycloalkyl_{(C≤12)}, alkoxy_{(C≤8)}, cycloalkoxy_{(C≤8)}, aryloxy_{(C≤12)}, acyloxy_{(C≤8)}, alkylamino_{(C≤8)}, cycloalkylamino_{(C≤8)}, dialkylamino_{(C≤8)}, arylamino_{(C≤8)}, aralkylamino_{(C≤8)}, alkylthio_{(C≤8)}, acylthio_{(C≤8)}, alkyl sulfonylamino_{(C≤8)}, cycloalkylsulfonylamino_{(C≤8)}, or substituted versions of any of these groups;
-alkanediyl_{(C≤8)}-R_{b}, -alkenediyl_{(C≤8)}-R_{b}, or a substituted version of any of these groups, wherein R_{b} is:
hydrogen, hydroxy, halo, amino or mercapto; or
heteroaryl_{(C≤8)}, alkoxy_{(C≤8)}, cycloalkoxy_{(C≤8)}, alkenyloxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, aralkylamino_{(C≤8)}, heteroarylamino_{(C≤8)}, alkylsulfonylamino_{(C≤8)}, cycloalkylsulfonylamino_{(C≤8)}, amido_{(C≤8)}, —OC(O)NH-alkyl_{(C≤8)}, or a substituted version of any of these groups;
—(CH₂)ₘC(O)R_{c}, wherein m is 0-6 and R_{c} is:
hydrogen, hydroxy, halo, amino, —NHOH, or mercapto; or
alkyl_{(C≤8)}, cycloalkyl_{(C≤8)}, alkenyl_{(C≤8)}, alkynyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, cycloalkoxy_{(C≤8)}, alkenyloxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, alkylsulfonylamino_{(C≤8)}, cycloalkylsulfonylamino_{(C≤8)}, amido_{(C≤8)}, —NH-alkoxy_{(C≤8)}, —NH-heterocycloalkyl_{(C≤8)}, —NH-amido_{(C≤8)}, or a substituted version of any of these groups;
R_{c} and R₃, taken together, are —O— or —NR_{d}—, wherein R_{d} is hydrogen or alkyl_{(C≤4)}; or
R_{c} and R₄, taken together, are —O— or —NR_{d}—, wherein R_{d} is hydrogen or alkyl_{(C≤4)}; or
—NHC(O)Rₑ, wherein Rₑ is:
hydrogen, hydroxy, amino; or
alkyl_{(C≤8)}, cycloalkyl_{(C≤8)}, alkenyl_{(C≤8)}, alkynyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, cycloalkoxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, or a substituted version of any of these groups;
or a pharmaceutically acceptable salt thereof,
wherein the patient has been identified as not having a history of left-sided heart failure or wherein the patient does not show evidence of left ventricular dysfunction.

6. The method of any one of claims 1-5, wherein the compound is further defined as: 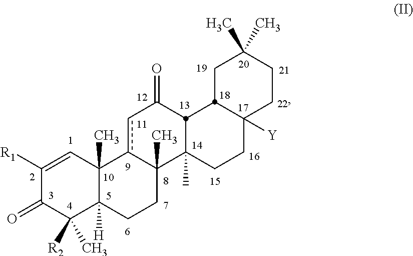
wherein:
R₁ is —CN, halo, —CF₃, or —C(O)Rₐ, wherein Rₐ is —OH, alkoxy_{(C1-4)}, —NH₂, alkylamino_{(C1-4)}, or —NH-S(O)₂-alkyl_{(C1-4)};
R₂ is hydrogen or methyl;
Y is:
—H, —OH, —SH, —CN, —F, —CF₃, —NH₂ or —NCO;
alkyl_{(C≤8)}, cycloalkyl_{(C≤8)}, alkenyl_{(C≤8)}, alkynyl_{(C≤8)}, aryl_{(C≤12)}, aralkyl_{(C≤12)}, heteroaryl_{(C≤8)}, heterocycloalkyl_{(C≤12)}, alkoxy_{(C≤8)}, cycloalkoxy_{(C≤8)}, aryloxy_{(C≤12)}, acyloxy_{(C≤8)}, alkylamino_{(C≤8)}, cycloalkylamino_{(C≤8)}, dialkylamino_{(C≤8)}, arylamino_{(C≤8)}, aralkylamino_{(C≤8)}, alkylthio_{(C≤8)}, acylthio_{(C≤8)}, alkyl sulfonylamino_{(C≤8)}, cycloalkylsulfonylamino_{(C≤8)}, or substituted versions of any of these groups;
-alkanediyl_{(C≤8)}-R_{b}, -alkenediyl_{(C≤8)}-R_{b}, or a substituted version of any of these groups, wherein R_{b} is:
hydrogen, hydroxy, halo, amino or mercapto; or
heteroaryl_{(C≤8)}, alkoxy_{(C≤8)}, cycloalkoxy_{(C≤8)}, alkenyloxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, aralkylamino_{(C≤8)}, heteroarylamino_{(C≤8)}, alkyl sulfonylamino_{(C≤8)}, cycloalkylsulfonylamino_{(C≤8)}, amido_{(C≤8)}, —OC(O)NH-alkyl_{(C≤8)}, or a substituted version of any of these groups;
—(CH₂)ₘC(O)R_{c}, wherein m is 0-6 and Rₑ is:
hydrogen, hydroxy, halo, amino, —NHOH, or mercapto; or
alkyl_{(C≤8)}, cycloalkyl_{(C≤8)}, alkenyl_{(C≤8)}, alkynyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, cycloalkoxy_{(C≤8)}, alkenyloxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, alkylsulfonylamino_{(C≤8)}, cycloalkylsulfonylamino_{(C≤8)}, amido_{(C≤8)}, —NH-alkoxy_{(C≤8)}, —NH— heterocycloalkyl_{(C≤8)}, —NH-amido_{(C≤8)}, or a substituted version of any of these groups;
—NHC(O)Rₑ, wherein Rₑ is:
hydrogen, hydroxy, amino; or
alkyl_{(C≤8)}, cycloalkyl_{(C≤8)}, alkenyl_{(C≤8)}, alkynyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, cycloalkoxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, or a substituted version of any of these groups;
or a pharmaceutically acceptable salt thereof.

7. The method of claim 6, wherein the compound is further defined as: 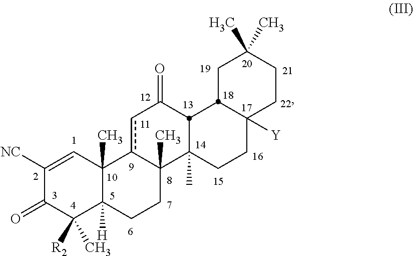
wherein:
R₂ is hydrogen or methyl;
Y is:
—H, —OH, —SH, —CN, —F, —CF₃, —NH₂ or —NCO;
alkyl_{(C≤8)}, cycloalkyl_{(C≤8)}, alkenyl_{(C≤8)}, alkynyl_{(C≤8)}, aryl_{(C≤12)}, aralkyl_{(C≤12)}, heteroaryl_{(C≤8)}, heterocycloalkyl_{(C≤12)}, alkoxy_{(C≤8)}, cycloalkoxy_{(C≤8)}, aryloxy_{(C≤12)}, acyloxy_{(C≤8)}, alkylamino_{(C≤8)}, cycloalkylamino_{(C≤8)}, dialkylamino_{(C≤8)}, arylamino_{(C≤8)}, aralkylamino_{(C≤8)}, alkylthio_{(C≤8)}, acylthio_{(C≤8)}, alkyl sulfonylamino_{(C≤8)}, cycl ° alkyl sulfonylamino_{(C≤8)}, or substituted versions of any of these groups;
-alkanediyl_{(C≤8)}-R_{b}, -alkenediyl_{(C≤8)}-R_{b}, or a substituted version of any of these groups, wherein R_{b} is:
hydrogen, hydroxy, halo, amino or mercapto; or
heteroaryl_{(C≤8)}, alkoxy_{(C≤8)}, cycloalkoxy_{(C≤8)}, alkenyloxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, aralkylamino_{(C≤8)}, heteroarylamino_{(C≤8)}, alkylsulfonylamino_{(C≤8)}, cycloalkylsulfylamino_{(C≤8)}, amido_{(C≤8)}, —OC(O)NH-alkyl_{(C≤8)}, or a substituted version of any of these groups;
—(CH₂)ₘC(O)R_{c}, wherein m is 0-6 and Rₑ is:
hydrogen, hydroxy, halo, amino, —NHOH, or mercapto; or
alkyl_{(C≤8)}, cycloalkyl_{(C≤8)}, alkenyl_{(C≤8)}, alkynyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, cycloalkoxy_{(C≤8)}, alkenyloxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, alkylsulfonylamino_{(C≤8)}, cycloalkylsulfonylamino_{(C≤8)}, amido_{(C≤8)}, —NH-alkoxy_{(C≤8)}, —NH— heterocycloalkyl_{(C≤8)}, —NH-amido_{(C≤8)}, or a substituted version of any of these groups;
—NHC(O)Rₑ, wherein Rₑ is:
hydrogen, hydroxy, amino; or
alkyl_{(C≤8)}, cycloalkyl_{(C≤8)}, alkenyl_{(C≤8)}, alkynyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, cycloalkoxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, or a substituted version of any of these groups;
or a pharmaceutically acceptable salt thereof.

8. The method of claim 7, wherein the compound is further defined as: 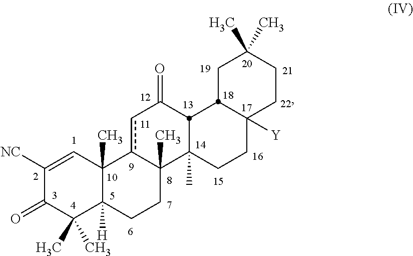
wherein:
Y is:
—H, —OH, —SH, —CN, —F, —CF₃, —NH₂ or —NCO;
alkyl_{(C≤8)}, cycloalkyl_{(C≤8)}, alkenyl_{(C≤8)}, alkynyl_{(C≤8)}, aryl_{(C≤12)}, aralkyl_{(C≤12)}, heteroaryl_{(C≤8)}, heterocycloalkyl_{(C≤12)}, alkoxy_{(C≤8)}, cycloalkoxy_{(C≤8)}, aryloxy_{(C≤12)}, acyloxy_{(C≤8)}, alkylamino_{(C≤8)}, cycloalkylamino_{(C≤8)}, dialkylamino_{(C≤8)}, arylamino_{(C≤8)}, aralkylamino_{(C≤8)}, alkylthio_{(C≤8)}, acylthio_{(C≤8)}, alkyl sulfonylamino_{(C≤8)}, cycloalkylsulfonylamino_{(C≤8)}, or substituted versions of any of these groups;
-alkanediyl_{(C≤8)}-R_{b}, -alkenediyl_{(C≤8)}-R_{b}, or a substituted version of any of these groups, wherein R^{b} is:
hydrogen, hydroxy, halo, amino or mercapto; or
heteroaryl_{(C≤8)}, alkoxy_{(C≤8)}, cycloalkoxy_{(C≤8)}, alkenyloxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, aralkylamino_{(C≤8)}, heteroarylamino_{(C≤8)}, alkyl sulfonylamino_{(C≤8)}, cycloalkylsulfonylamino_{(C≤8)}, amido_{(C≤8)}, —OC(O)NH-alkyl_{(C≤8)}, or a substituted version of any of these groups;
—(CH₂)ₘC(O)R_{c}, wherein m is 0-6 and R_{c} is:
hydrogen, hydroxy, halo, amino, —NHOH, or mercapto; or
alkyl_{(C≤8)}, cycloalkyl_{(C≤8)}, alkenyl_{(C≤8)}, alkynyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, cycloalkoxy_{(C≤8)}, alkenyloxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, alkylsulfonylamino_{(C≤8)}, cycloalkylsulfonylamino_{(C≤8)}, amido_{(C≤8)}, —NH-alkoxy_{(C≤8)}, —NH— heterocycloalkyl_{(C≤8)}, —NH-amido_{(C≤8)}, or a substituted version of any of these groups;
—NHC(O)Rₑ, wherein Rₑ is:
hydrogen, hydroxy, amino; or
alkyl_{(C≤8)}, cycloalkyl_{(C≤8)}, alkenyl_{(C≤8)}, alkynyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, cycloalkoxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, or a substituted version of any of these groups;
or a pharmaceutically acceptable salt thereof.

9. The method of claim 8, wherein the compound is further defined as: 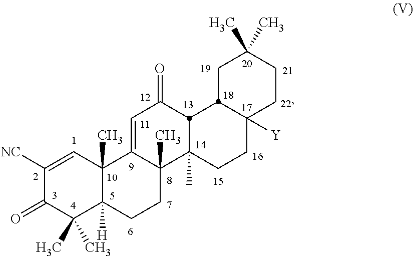
wherein:
Y is:
—H, —OH, —SH, —CN, —F, —CF₃, —NH₂ or —NCO;
alkyl_{(C≤8)}, cycloalkyl_{(C≤8)}, alkenyl_{(C≤8)}, alkynyl_{(C≤8)}, aryl_{(C≤12)}, aralkyl_{(C≤12)}, heteroaryl_{(C≤8)}, heterocycloalkyl_{(C≤12)}, alkoxy_{(C≤8)}, cycloalkoxy_{(C≤8)}, aryloxy_{(C≤12)}, acyloxy_{(C≤8)}, alkylamino_{(C≤8)}, cycloalkylamino_{(C≤8)}, dialkylamino_{(C≤8)}, arylamino_{(C≤8)}, aralkylamino_{(C≤8)}, alkylthio_{(C≤8)}, acylthio_{(C≤8)}, alkyl sulfonylamino_{(C≤8)}, cycloalkylsulfonylamino_{(C≤8)}, or substituted versions of any of these groups;
-alkanediyl_{(C≤8)}-R_{b}, -alkenediyl_{(C≤8)}-R_{b}, or a substituted version of any of these groups, wherein R_{b} is:
hydrogen, hydroxy, halo, amino or mercapto; or
heteroaryl_{(C≤8)}, alkoxy_{(C≤8)}, cycloalkoxy_{(C≤8)}, alkenyloxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, aralkylamino_{(C≤8)}, heteroarylamino_{(C≤8)}, alkyl sulfonylamino_{(C≤8)}, cycloalkylsulfonylamino_{(C≤8)}, amido_{(C≤8)}, —OC(O)NH-alkyl_{(C≤8)}, or a substituted version of any of these groups;
—(CH₂)ₘC(O)R_{c}, wherein m is 0-6 and Rₑ is:
hydrogen, hydroxy, halo, amino, —NHOH, or mercapto; or
alkyl_{(C≤8)}, cycloalkyl_{(C≤8)}, alkenyl_{(C≤8)}, alkynyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, cycloalkoxy_{(C≤8)}, alkenyloxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, alkylsulfonylamino_{(C≤8)}, cycloalkylsulfonylamino_{(C≤8)}, amido_{(C≤8)}, —NH-alkoxy_{(C≤8)}, —NH— heterocycloalkyl_{(C≤8)}, —NH-amido_{(C≤8)}, or a substituted version of any of these groups;
—NHC(O)Rₑ, wherein Rₑ is:
hydrogen, hydroxy, amino; or
alkyl_{(C≤8)}, cycloalkyl_{(C≤8)}, alkenyl_{(C≤8)}, alkynyl_{(C≤8)}, aryl_{(C≤8)}, aralkyl_{(C≤8)}, heteroaryl_{(C≤8)}, heterocycloalkyl_{(C≤8)}, alkoxy_{(C≤8)}, cycloalkoxy_{(C≤8)}, aryloxy_{(C≤8)}, aralkoxy_{(C≤8)}, heteroaryloxy_{(C≤8)}, acyloxy_{(C≤8)}, alkylamino_{(C≤8)}, cycloalkylamino_{(C≤8)}, dialkylamino_{(C≤8)}, arylamino_{(C≤8)}, or a substituted version of any of these groups;
or a pharmaceutically acceptable salt thereof.

10. The method of claim 9, wherein the compound is further defined as:

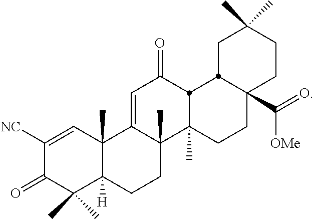

11. The method of any one of claims 1-9, wherein the compound is not

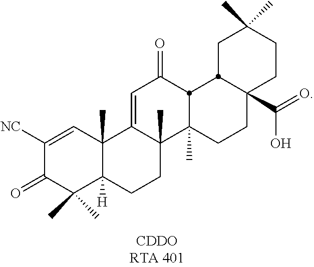

12. The method according to any one of claims 1-11, wherein the patient does not have cardiovascular disease.

13. The method according to any one of claims 1-11, wherein the patient has cardiovascular disease.

14. The method according to either claim 12 or claim 13, wherein the cardiovascular disease is left-sided myocardial disease.

15. The method according to either claim 12 or claim 13, wherein the cardiovascular disease is atherosclerosis.

16. The method according to either claim 12 or claim 13, wherein the cardiovascular disease is restenosis.

17. The method according to either claim 12 or claim 13, wherein the cardiovascular disease is thrombosis.

18. The method according to either claim 12 or claim 13, wherein the cardiovascular disease is pulmonary hypertension.

19. The method of claim 18, wherein the pulmonary hypertension is World Health Organization (WHO) Class I pulmonary hypertension (pulmonary arterial hypertension or PAH).

20. The method of claim 19, wherein the pulmonary hypertension is pulmonary arterial hypertension associated with connective tissue disease.

21. The method of claim 19, wherein the pulmonary hypertension is idiopathic pulmonary arterial hypertension.

22. The method of claim 18, wherein the pulmonary hypertension is WHO Class II pulmonary hypertension.

23. The method of claim 18, wherein the pulmonary hypertension is WHO Class III pulmonary hypertension.

24. The method of claim 18, wherein the pulmonary hypertension is WHO Class IV pulmonary hypertension.

25. The method of claim 18, wherein the pulmonary hypertension is WHO Class V pulmonary hypertension.

26. The method according to any one of claims 1-25, wherein the patient does not have endothelial dysfunction.

27. The method according to any one of claims 1-25, wherein the patient has endothelial dysfunction.

28. The method according to any one of claims 1-27, wherein the patient does not have Stage 4 or higher chronic kidney disease.

29. The method according to any one of claims 1-27, wherein the patient does not have an estimated glomerular filtration rate (eGFR) less than 45 mL/min/1.73 m².

30. The method according to any one of claims 1-27, wherein the patient does not have an elevated albumin/creatinine ratio (ACR) is greater than 2000 mg/g.

31. The method according to any one of claims 1-30, wherein the patient does not have diabetes.

32. The method according to any one of claims 1-30, wherein the patient has diabetes.

33. The method of either claim 31 or claim 32, wherein the diabetes is Type 2 diabetes.

34. The method according to any one of claims 1-33, wherein the patient does not have a complication associated with diabetes.

35. The method according to any one of claims 1-33, wherein the patient has a complication associated with diabetes.

36. The method according to either claim 34 or claim 35, wherein the complication associated with diabetes is diabetic nephropathy.

37. The method according to either claim 34 or claim 35, wherein the complication is selected from the group consisting of obesity, stroke, peripheral vascular disease, neuropathy, myonecrosis, retinopathy and metabolic syndrome (syndrome X).

38. The method according to any one of claims 1-37, wherein the patient does not have insulin resistance.

39. The method according to any one of claims 1-37, wherein the patient has insulin resistance.

40. The method according to any one of claims 1-39, wherein the patient does not have fatty liver disease.

41. The method according to any one of claims 1-39, wherein the patient has fatty liver disease.

42. The method according to any one of claims 1-39, wherein the patient does not have hepatic impairment.

43. The method according to any one of claims 1-39, wherein the patient has hepatic impairment.

44. The method according to any one of claims 1-43, wherein the patient is not overweight.

45. The method according to any one of claims 1-43, wherein the patient is overweight.

46. The method according to either claim 44 or claim 45, where the patient is obese.

47. The method of claim 46, where the obesity is class I.

48. The method of claim 46, where the obesity is class II.

49. The method of claim 46, where the obesity is class III.

50. The method according to either claim 45 or claim 46, where the patient's body mass index (BMI) is from 25 kg/m² to 30 kg/m².

51. The method according to either claim 45 or claim 46, where the patient's BMI is from kg/m² to 35 kg/m².

52. The method according to either claim 45 or claim 46, where the patient's BMI is from kg/m² to 40 kg/m².

53. The method according to either claim 45 or claim 46, where the patient's BMI is from kg/m² to 80 kg/m².

54. The method according to any one of claims 1-41, wherein the patient has cancer.

55. The method according to any one of claims 1-41, wherein the patient does not have cancer.

56. The method of either claim 54 or claim 55, wherein the cancer is an advanced solid tumor or lymphoid malignancy.

57. The method of either claim 54 or claim 55, wherein the cancer is selected from the groups consisting of breast cancer, prostate cancer, colon cancer, brain cancer, melanoma, pancreatic cancer, ovarian cancer, leukemia, or bone cancer.

58. The method of claim 57, wherein the cancer is an advanced malignant melanoma.

59. The method of claim 57, wherein the cancer is pancreatic cancer.

60. The method according to any one of claims 1-59, wherein the patient does not have chronic obstructive pulmonary disease (COPD).

61. The method according to any one of claims 1-59, wherein the patient has chronic obstructive pulmonary disease (COPD).

62. The method according to any one of claims 1-61, wherein the patient is a smoker.

63. The method according to any one of claims 1-61, wherein the patient is not a smoker.

64. The method according to any one of claims 1-63, wherein the patient has impaired renal function.

65. The method according to any one of claims 1-64, wherein the patient has elevated levels of at least one biomarker associated with renal disease.

66. The method of claim 65, wherein the biomarker is serum creatinine.

67. The method of claim 65, wherein the biomarker is cystatin C.

68. The method of claim 65, wherein the biomarker is uric acid.

69. The method according to any one of claims 1-68, wherein the patient has radiographic infiltrates by imaging prior to treatment.

70. The method of claim 69, wherein the imaging is a chest X-ray or CT scan.

71. The method according to any one of claims 1-70, wherein the patient's blood oxygen saturation, at rest, is at most 94% prior to treatment.

72. The method according to any one of claims 1-71, wherein the patient requires supplemental oxygen prior to treatment.

73. The method according to any one of claims 1-72, wherein the patient requires non-invasive ventilation prior to treatment.

74. The method according to any one of claims 1-73, wherein the patient requires mechanical ventilation for up to 2 days prior to treatment.

75. The method according to any one of claims 1-68, wherein the method is a method of treating or preventing COVID-19 in a patient in need thereof.

76. The method according to any one of claim 1-68, wherein the coronavirus is a betacoronavirus.

77. The method according to any one of claims 1-68, wherein the coronavirus is a severe acute respiratory syndrome-related (SARS) coronavirus.

78. The method according to any one of claims 1-75, wherein the coronavirus is SARS-CoV-2.

79. The method according to any one of claims 1-78, wherein the method inhibits replication of SARS-CoV-2 in the patient.

80. The method according to any one of claims 1-79, wherein the method suppresses or prevents systemic inflammation in the patient.

81. The method of claim 80, wherein the method suppresses or prevents a cytokine storm in the patient.

82. The method according to any one of claims 1-80, wherein the method suppresses or prevents lung inflammation in the patient.

83. The method according to any one of claims 1-82, wherein the method treats or prevents inflammation-induced liver damage in the patient.

84. The method according to any one of claims 1-83, wherein the method treats or prevents acute lung injury in the patient.

85. The method according to any one of claims 1-84, wherein the method treats or prevents kidney damage in the patient.

86. The method according to any one of claims 1-85, wherein the method treats or prevents acute kidney injury in the patient.

87. The method according to any one of claims 1-86, wherein the method improves kidney function in the patient.

88. The method according to any one of claims 1-87, wherein the method increases the patient's estimated glomerular filtration rate (eGFR).

89. The method according to any one of claims 1-88, wherein the method treats or prevents acute respiratory distress syndrome in the patient.

90. The method according to any one of claims 1-89, wherein the method treats or prevents seizures in the patient.

91. The method according to any one of claims 1-90, wherein the method treats or prevents brain inflammation in the patient.

92. The method according to any one of claims 1-91, wherein the method reduces the hospitalization time of the patient.

93. The method according to any one of claims 1-92, wherein the method reduces the time spent in the intensive care unit.

94. The method according of any one of claims 1-91, wherein the method delays the need for hospitalization of the patient.

95. The method according of any one of claims 1-94, wherein the method increases the probability of survival of the patient.

96. The method according of any one of claims 1-95, wherein the method prevents the need for non-invasive mechanical ventilation.

97. The method according of any one of claims 1-96, wherein the method prevents the need for invasive mechanical ventilation.

98. The method according of any one of claims 1-97, wherein the method prevents respiratory failure.

99. The method according to any one of claims 1-98, wherein the method lowers the patient's WHO score.

100. The method according of any one of claims 1-99, wherein the method prevents the need for renal replacement therapy.

101. The method according to any one of claims 1-100, wherein the patient exhibits microhematuria.

102. The method of claim 101, wherein the patient exhibits hematuria.

103. The method according to any one of claims 1-102, wherein the patient further exhibits microalbuminuria.

104. The method of claim 103, wherein the patient exhibits albuminuria.

105. The method of either claim 103 or claim 104, wherein the concentration of albumin in the urine is between 30 μg per mg of creatinine and 300 μg per mg of creatinine.

106. The method of either claim 103 or claim 104, wherein the concentration of albumin in the urine is greater than 300 μg per mg of creatinine.

107. The method according to any one of claims 1-106, wherein the patient further exhibits proteinuria.

108. The method of claim 107, wherein the patient exhibits overt proteinuria.

109. The method of either claim 107 or claim 108, wherein the patient's urine exhibits the presence of multiple proteins.

110. The method according to any one of claims 107-109, wherein the patient's urine exhibits a protein to creatinine ratio of greater than 0.2 mg/g.

111. The method of claim 110, wherein the patient's urine exhibits a protein to creatinine ratio of greater than 1.0 mg/g.

112. The method according to any one of claims 1-111, wherein the patient has an eGFR less than 45 mL/min/1.73 m².

113. The method of claim 112, wherein the patient exhibits an ACR of less than 2000 mg/g.

114. The method according to any one of claims 1-113, wherein the patient is less than 75 years old.

115. The method of claim 114, wherein the patient is less than 70 years old.

116. The method of claim 114, wherein the patient is less than 60 years old.

117. The method of claim 116, wherein the patient is less than 40 years old.

118. The method of claim 117, wherein the patient is less than 30 years old.

119. The method of claim 118, wherein the patient is less than 25 years old.

120. The method according to any one of claims 1-119, wherein the patient does not have at least one of the following characteristics:
(A) a cardiovascular disease;
(B) an elevated baseline B-type natriuretic peptide (BNP) level;
(C) an estimated glomerular filtration rate (eGFR)<45 mL/min/1.73 m²; and
(D) an elevated albumin/creatinine ratio (ACR)>2000 mg/g.

121. The method of claim 120, wherein the patient does not have two of the characteristics.

122. The method of claim 120, wherein the patient does not have three of the characteristics.

123. The method of claim 122, wherein the patient does not have any of said characteristics.

124. The method according to any one of claims 1-123, wherein the patient has not been intubated for three or more days at the time of treatment.

125. The method according to any one of claims 1-124, wherein the patient has not been on mechanical ventilation for three or more days at the time of treatment.

126. The method according to any one of claims 1-124, wherein the patient has not been on invasive mechanical ventilation for three or more days at the time of treatment.

127. The method according to any one of claims 1-124, wherein the patient has not been intubated for three or more days at the time of treatment.

128. The method according to any one of claims 1-127, wherein the patient is not known to have an impaired left ventricular ejection fraction, preferably wherein the patient is not known to have a left ventricular ejection fraction (LVEF) of less than 40%.

129. The method according to any one of claims 1-128, wherein the patient has not been previously hospitalized for heart failure.

130. The method according to any one of claims 1-129, wherein the patient has not previously experienced cardiac arrest.

131. The method according to any one of claims 1-130, wherein the patient has not previously experienced shock.

132. The method according to any one of claims 1-131, wherein the patient has not does not have an uncontrolled bacterial infection.

133. The method according to any one of claims 1-132, wherein the patient has not does not have an uncontrolled fungal infection.

134. The method according to any one of claims 1-133, wherein the patient has not does not have another uncontrolled viral infection in addition to the SARS-CoV-2 infection.

135. The method according to any one of claims 1-134, wherein the patient has not does not have a history of cirrhosis, chronic active hepatitis, or severe hepatic disease.

136. The method according to any one of claims 1-135, wherein the patient does not have a history of an estimated glomerular filtration rate (eGFR)<15 mL/min/1.73 m².

137. The method according to any one of claims 1-136, wherein the patient does not have a history of requiring dialysis.

138. The method according to any one of claims 1-137, wherein the patient does not have an ALT level or AST level that is more than 5 times greater than the ULN.

139. The method according to any one of claims 1-138, wherein the patient is a human.

140. The method according to any one of claims 1-139, wherein the patient is male.

141. The method according to any one of claims 1-139, wherein the patient is female.

142. The method according to any one of claims 10-141, wherein at least a portion of the compound is present as a crystalline form having an X-ray diffraction pattern (CuKα) comprising significant diffraction peaks at about 8.8, 12.9, 13.4, 14.2 and 17.4 °2θ.

143. The method of claim 142, wherein the X-ray diffraction pattern (CuKα) is substantially as shown in FIG. 1A or FIG. 1B.

144. The method according to any one of claims 10-141, wherein at least a portion of the compound is present as an amorphous form having an X-ray diffraction pattern (CuKα) with a peak at approximately 13.5 °2θ, substantially as shown in FIG. 1C, and a transition glass temperature (T_{g}).

145. The method of claim 144, wherein the T_{g} value is in the range of about 120° C. to about 135° C.

146. The method of claim 145, wherein the T_{g} value is in the range of about 125° C. to about 130° C.

147. The method according to any one of claims 10-141, wherein at least a portion of the compound is present as a crystalline form having an X-ray diffraction pattern (CuKα) comprising significant diffraction peaks at about 6.2, 12.4, 15.4, 18.6 and 24.9 °2θ.

148. The method of claim 147, wherein the crystalline form is further characterized by one, two, three, four or five additional diffraction peaks selected from the group consisting of 8.6, 13.3, 13.7, 17.1 and 21.7 °2θ.

149. The method according to any one of claims 10-141, wherein at least a portion of the compound is present as a crystalline form having an X-ray diffraction pattern (CuKα) comprising significant diffraction peaks at about 3.6, 7.1, 10.8, 12.4 and 16.5 °2θ.

150. The method of claim 149, wherein the crystalline form is further characterized by one, two, three, four or five additional diffraction peaks selected from the group consisting of 12.9, 13.9, 14.8, 18.6 and 20.6 °2θ.

151. The method of either claim 149 or 150, wherein the crystalline form is further characterized by a Raman spectrum having peaks at 2949, 1671, 1618 and 1464±4 cm⁻¹.

152. The method according to any one of claims 10-141, wherein at least a portion of the compound is present as a toluene solvate crystalline form having an X-ray diffraction pattern (CuKα) comprising diffraction peaks at about 9.65, 7.58, 7.18, 6.29, 6.06, 5.47, 5.21, 4.77 and 3.07 °2θ.

153. The method according to any one of claims 10-141, wherein at least a portion of the compound is present as a semi-dioxane solvate crystalline form having an X-ray diffraction pattern (CuKα) comprising diffraction peaks at about 10.01, 7.09, 6.84, 6.23, 5.29, 5.20, 5.10, 4.84, and 4.61 °2θ.

154. The method according to any one of claims 10-141, wherein at least a portion of the compound is present as a semi-tetrahydrofuran solvate crystalline form having an X-ray diffraction pattern (CuKα) comprising diffraction peaks at about 10.00, 7.14, 6.80, 6.65, 6.10, 5.62, 5.29, 4.88, and 4.50 °2θ.

155. The method according to any one of claims 10-141, wherein at least a portion of the compound is present as a methanol solvate crystalline form having an X-ray diffraction pattern (CuKα) comprising diffraction peaks at about 8.86, 8.45, 8.17, 7.90, 7.26, 4.67, 6.63, 6.46, and 3.64 °2θ.

156. The method according to any one of claims 10-141, wherein at least a portion of the compound is present as an anhydrous crystalline form having an X-ray diffraction pattern (CuKα) comprising diffraction peaks at about 12.05, 8.90, 8.49, 8.13, 7.92, 7.29, 6.64, 4.67 and 3.65 °2θ.

157. The method according to any one of claims 10-141, wherein at least a portion of the compound is present a dihydrate crystalline form having an X-ray diffraction pattern (CuKα) comprising diffraction peaks at about 8.81, 8.48, 7.91, 7.32, 5.09, 4.24, 3.58, 3.36 and 3.17 °2θ.

158. The method according to any one of claims 1-157, wherein the therapeutically effective amount is a daily dose from about 0.1 mg to about 300 mg of the compound.

159. The method of claim 158, wherein the daily dose is from about 0.5 mg to about 200 mg of the compound.

160. The method of claim 159, wherein the daily dose is from about 1 mg to about 150 mg of the compound.

161. The method of claim 160, wherein the daily dose is from about 1 mg to about 75 mg of the compound.

162. The method of claim 161, wherein the daily dose is from about 1 mg to about 20 mg of the compound.

163. The method of claim 158, wherein the daily dose is from about 2.5 mg to about 30 mg of the compound.

164. The method of claim 163, wherein the daily dose is about 2.5 mg of the compound.

165. The method of claim 163, wherein the daily dose is about 5 mg of the compound.

166. The method of claim 163, wherein the daily dose is about 10 mg of the compound.

167. The method of claim 163, wherein the daily dose is about 15 mg of the compound.

168. The method of claim 163, wherein the daily dose is about 20 mg of the compound.

169. The method of claim 163, wherein the daily dose is about 30 mg of the compound.

170. The method according to any of claims 1-157, wherein the therapeutically effective amount is a daily dose is 0.01-100 mg of compound per kg of body weight.

171. The method of claim 170, wherein the daily dose is 0.05-30 mg of compound per kg of body weight.

172. The method of claim 171, wherein the daily dose is 0.1-10 mg of compound per kg of body weight.

173. The method of claim 172, wherein the daily dose is 0.1-5 mg of compound per kg of body weight.

174. The method of claim 173, wherein the daily dose is 0.1-2.5 mg of compound per kg of body weight.

175. The method according to any of claims 1-157, wherein the compound is administered as a single dose to the patient per day.

176. The method according to any of claims 1-157, wherein the compound is administered as two or more doses to the patient per day.

177. The method according to any one of claims 1-157, wherein the compound is administered orally, intraarterially or intravenously.

178. The method according to any one of claims 1-157, wherein the compound is formulated as a hard or soft capsule or a tablet.

179. The method according to any one of claims 1-157, wherein the compound is formulated as a solid dispersion comprising (i) the compound and (ii) an excipient.

180. The method of claim 179, wherein the excipient is a methacrylic acid-ethyl acrylate copolymer.

181. The method of claim 180, wherein the copolymer comprises methacrylic acid and ethyl acrylate at a 1:1 ratio.

182. The method according to any of claims 1-181, further comprising administering to patient a second therapy.

183. The method of claim 182, wherein the second therapy comprises administering to said patient a therapeutically effective amount of a second drug or of convalescent plasma.

184. The method of claim 183, wherein the second drug is an anti-platelet drug, an anti-coagulation agent, an anti-viral drug, a corticosteroid, or a human type I IFN.

185. The method of claim 183, wherein the second drug is remdesivir or tocilizumab.
